(19) United States
(12) Patent Application Publication
(10) Pub. No.: US 2014/0113898 A1
(43) Pub. Date: Apr. 24, 2014

(54) BISARYLSULFONE AND DIALKYLARYLSULFONE COMPOUNDS AS CALCIUM CHANNEL BLOCKERS

(71) Applicants: Hassan A. Pajouhesh, West Vancouver (CA); Richard Holland, Vancouver (CA); Yuanxi Zhou, Richmond (CA); Yongbao Zhu, Langley (CA); Michael Edward Grimwood, North Vancouver (CA); Navjot Chahal, Vancouver (CA)

(72) Inventors: Hassan A. Pajouhesh, West Vancouver (CA); Richard Holland, Vancouver (CA); Yuanxi Zhou, Richmond (CA); Yongbao Zhu, Langley (CA); Michael Edward Grimwood, North Vancouver (CA); Navjot Chahal, Vancouver (CA)

(73) Assignee: Zalicus Pharmaceuticals Ltd., Cambrisge, MA (US)

(21) Appl. No.: 13/883,956

(22) PCT Filed: Nov. 4, 2011
(86) PCT No.: PCT/CA11/01240

§ 371 Date: Jan. 9, 2014

Related U.S. Application Data
(60) Provisional application No. 61/410,966, filed on Nov. 8, 2010.
(60) Provisional application No. 61/410,954, filed on Nov. 8, 2010.

(51) Int. Cl. C07D 487/04; C07D 213/82; C07D 295/26; C07D 239/28; C07D 471/04; C07C 317/32; C07D 231/14

(52) U.S. Cl. CPC C07D 487/04; C07C 317/32; C07D 213/82; C07D 231/14; C07D 239/28; C07D 471/04; C07D 295/26
USPC 514/238.2; 564/185; 514/617; 546/316; 514/355; 546/291; 514/346; 548/374.1; 514/406; 544/335; 514/256; 564/162; 514/618; 546/121; 514/300; 544/350; 514/249; 544/159

(57) ABSTRACT

The invention relates to bisarylsulfone and dialkylarylsulfone compounds (e.g., compounds according to any of Formulas (I)-(IX) or compounds (1)-(227) of Tables 4 and 5) useful in treating conditions associated with calcium channel function, and particularly conditions associated with N-type calcium channel activity. The invention also relates to pharmaceutical compositions that include these bisarylsulfone compounds, as well methods for the treatment of conditions such as cardiovascular disease, epilepsy, cancer and pain.

### CROSS REFERENCE TO RELATED APPLICATIONS

This application claims benefit of U.S. Provisional Application Nos. 61/410,954 and 61/410,966, both of which were filed on Nov. 8, 2010, and are hereby incorporated by reference in their entirety.

### BACKGROUND OF THE INVENTION

The invention relates to compounds useful in treating conditions associated with calcium channel function, and particularly conditions associated with N- and/or T-type calcium channel activity. More specifically, the invention concerns compounds containing bisarylsulfone and dialkylarylsulfone compounds that are useful in treatment of conditions such as cardiovascular disease, epilepsy, cancer and pain.

Calcium channels mediate a variety of normal physiological functions and are also implicated in a number of human disorders. Examples of calcium-mediated human disorders include but are not limited to congenital migraine, cerebellar ataxia, angina, epilepsy, hypertension, ischemia, and some arrhythmias (see, e.g., Janis et al., Ion Calcium Channels: Their Properties, Functions, Regulation and Clinical Relevance (1991) CRC Press, London

T-type, or low voltage-activated, channels describe a broad class of molecules that transiently activate at negative potentials and are highly sensitive to changes in resting potential and are involved in various medical conditions. For example, in mice lacking the gene expressing the 3.1 subunit (Ca_{V} 3.1), resistance to absence seizures was observed (Kim et al., Mol Cell Neurosci 18(2): 235-245, 2001). Other studies have also implicated the 3.2 subunit (Ca_{V} 3.2) in the development of epilepsy (Su et al., J Neurosci 22: 3645-3655, 2002).

Novel allosteric modulators of calcium channels, e.g., N- or T-type calcium channels, are thus desired. Modulators may affect the kinetics and/or the voltage potentials of e.g., the Ca_{V}3.1, Ca_{V}3.2, Ca_{V}3.3, or Ca_{V}2.2 channel.

The invention provides compounds that act at these N- and/or T-type calcium channels and are useful to treat various conditions associated with these calcium channels, such as pain and epilepsy. It also provides pharmaceutical compositions containing these compounds and methods to use them either alone or in combination with other pharmaceutical agents.

### SUMMARY OF THE INVENTION

The invention provides compounds that act at, e.g., N- and/or T-type calcium channels and are useful to treat various conditions associated with these calcium channels, such as pain and epilepsy. It also provides pharmaceutical compositions containing these compounds and methods to use them either alone or in combination with other pharmaceutical agents.

In a first aspect, the invention features a compound having a structure according to the following formula,

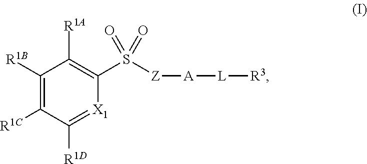

or a pharmaceutically acceptable salt, solvate, or prodrug thereof, or a stereoisomer thereof, where

X¹ is N or CR^{1E};

each of R^{1A}, R^{1B}, R^{1C}, R^{1D}, and R^{1E} is selected, independently, from H, OH, halogen, optionally substituted C1-C6 alkyl, optionally substituted C2-C6 alkenyl, optionally substituted C2-C6 alkynyl, and optionally substituted C1-C6 alkoxy;

Z is —(CR^{Z1}R^{Z2})R^{Z3}—, optionally substituted phenyl, or optionally substituted pyridyl;

each of R^{Z1} and R^{Z2} is, independently optionally substituted C1-C6 alkyl;

R^{Z3} is a covalent bond or an unsubstituted C1-C3 alkylene;

A is a covalent bond or an optionally substituted C1-C3 alkylene;

L is —CONR^{2A}(CH₂)ₒ or —R^{2A}NCO(CH₂)ₒ, where R^{2A} is H or optionally substituted C1-C6 alkyl, and o is 0, 1, or 2; and

R³ is selected from optionally substituted C1-C6 alkyl, optionally substituted alkaryl, optionally substituted alkheteroaryl, optionally substituted aryl, optionally substituted heteroaryl, optionally substituted C3-C9 cycloalkyl, and optionally substituted heterocyclyl.

In some embodiments, A is a covalent bond or an optionally substituted C1 alkylene.

In certain embodiments, the compound has a structure according to the following formula,

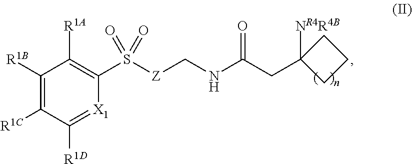

where R^{4A} and R^{4B} are each, independently, H or optionally substituted C1-C6 alkyl, and n is an integer between 0-4.

In other embodiments, R^{4A} and R^{4B} are both H, and/or n is 2.

In still other embodiments, X¹ is CH and one or two of R^{1A}, R^{1B}, R^{1C}, R^{1D}, and R^{1E} are independently, halogen, C1 haloalkyl or C1 haloalkoxy.

In particular embodiments, R^{1A}, R^{1D}, and R^{1E} are each H, and R^{1B} and R^{1C} are, independently, H, CF₃, or OCF₃.

In some embodiments, Z is C(CH₃)₂(CH₂)₂, unsubstituted phenyl, unsubstituted pyridyl, or a substituted phenyl or pyridyl group including 1-4 substituents selected, independently, from OH, optionally substituted C1-C6 alkyl, optionally substituted C2-C6 alkenyl, optionally substituted C2-C6 alkynyl, or optionally substituted C1-C6 alkoxy, optionally substituted aryloxy, or optionally substituted heteroaryloxy.

In certain embodiments, the optionally substituted aryloxy includes a phenyl group having zero, one, or two substituents that are, independently, halogen, C1 haloalkyl, or C1 haloalkoxy, or the optionally substituted heteroaryloxy includes a pyridyl group having zero, one, or two substituents that are, independently, halogen, C1 haloalkyl, or C1 haloalkoxy.

In still other embodiments, the compound has a structure according to the following formula,

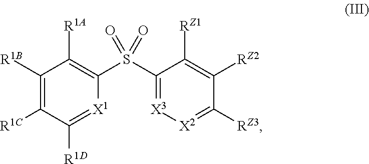

or a pharmaceutically acceptable salt, solvate, or prodrug thereof, or a stereoisomer thereof, where

X¹ is N or CR^{1E};

X² is N or CR^{Z4};

X³ is N or CR^{z5};

each of R^{1A}, R^{1B}, R^{1C}, R^{1D}, R^{1E}, R^{Z4}, and R^{Z5} is selected, independently, from H, OH, optionally substituted C1-C6 alkyl, optionally substituted C2-C6 alkenyl, optionally substituted C2-C6 alkynyl, and optionally substituted C1-C6 alkoxy;

each of R^{Z1}, R^{Z2}, and R^{Z3} is selected, independently, from H, OH, optionally substituted C1-C6 alkyl, optionally substituted C2-C6 alkenyl, optionally substituted C2-C6 alkynyl, optionally substituted C1-C6 alkoxy, or the substructure ALR³, and where one and only one of R^{Z1}, R^{Z2}, and R^{Z3} is the substructure ALR³;

and

where no more than one of X² and X³ is N.

In some embodiments, R^{1B} is C1-C6 haloalkyl or C1-C6 haloalkoxy, preferably R^{1B} is CF₃ or OCF₃.

In other embodiments, X² or X³ is N.

In still other embodiments, A is CH₂.

In certain embodiments, L is —NHCO—, —CONH—, —NHCOCH₂—, or —CONHCH₂—.

In particular embodiments, R³ is substituted C1-C6 alkyl, substituted aryl, substituted heteroaryl, substituted heterocyclyl, and substituted C3-C9 cycloalkyl, preferably R³ includes a substituent selected from CF₃, OCF₃, F, Cl, OH, —SO₂Me, —SO₂ⁱPr, and NH₂.

In some embodiments, the compound, or a pharmaceutically acceptable salt, solvate, or prodrug thereof, or a stereoisomer thereof, has a structure according to one of the following formulas,

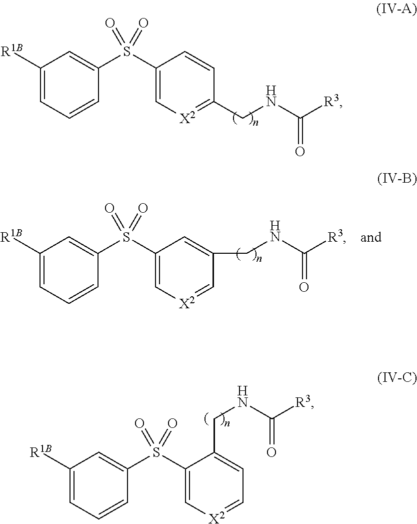

where

X² is N or CH;

R^{1B} is C1-C3 haloalkyl or C1-C3 haloalkoxy;

n is 1, 2, or 3; and

R³ is C1-C3 haloalkyl, optionally substituted heterocyclyl, optionally substituted aryl, optionally substituted heteroaryl, optionally substituted aryl, optionally substituted benzyl, or optionally substituted C3-C9 cycloalkyl.

In some embodiments, R³ is C1-C3 haloalkyl.

In other embodiments, R³ is optionally substituted piperidinyl, optionally substituted tetrahydropyranyl, optionally substituted pyrrolidinyl, optionally substituted cyclopropyl, optionally substituted cyclobutyl, or optionally substituted cyclohexyl.

In still other embodiments, R³ is substituted and selected from pyridyl, pyrimidyl, pyrazolyl, benzimidazolyl, imidazo[1,2-a]pyridyl, and 6,7-dihydropyrazolo[1,5-a]pyrazin-4(5H)-one.

In certain embodiments, R³ is optionally substituted phenyl.

In other embodiments, R³ is unsubstituted, or R³ includes 1, 2, or 3 substituents selected, independently, from OH, NH₂, F, Cl, CH₃, C1-C3 haloalkyl, C1-C3 haloalkoxy, SO₂ (optionally substituted C1-C4 alkyl), SO₂ (optionally substituted aryl), and unsubstituted C3-C6 cycloalkyl.

In some embodiments, X² is N.

In other embodiments, X² is CH.

In still other embodiments, the compound, or a pharmaceutically acceptable salt, solvate, or prodrug thereof, or a stereoisomer thereof, has a structure according to the following formula,

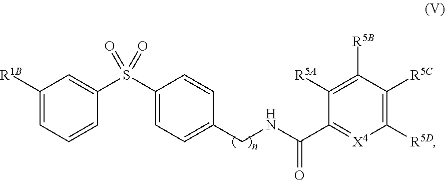

where

n is 1 or 2;

X⁴ is N or CH; and

each of R^{5A}, R^{5B}, R^{5C}, and R^{5D} is selected, independently, from H, F, Cl, C1-C3 haloalkyl, C1-C3 haloalkoxy, and SO₂(C1-C4 alkyl).

In some embodiments, n is 1.

In other embodiments, X⁴ is N.

In certain embodiments, each of R^{5A}, R^{5B}, R^{5C}, and R^{5D} is selected, independently, from H, F, Cl, CF₃, OCF₃, SO₂Me, and SO₂ⁱPr.

In still other embodiments, R^{1B} is CF₃ or OCF₃.

In a second aspect, the invention features a compound having a structure according to the following formula,

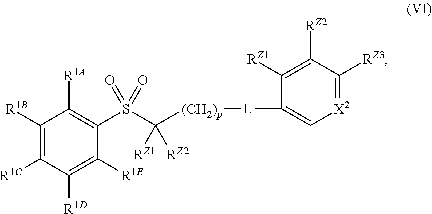

or a pharmaceutically acceptable salt, solvate, or prodrug thereof, or a stereoisomer thereof, where

p is 0, 1, 2, or 3;

L is —C(O)NR^{2A}- or —NR^{2A}C(O)—;

each of R^{Z1} and R^{Z2} is, independently, optionally substituted C1-C6 alkyl;

R^{2A} is H or optionally substituted C1-C6 alkyl;

each of R^{1A}, R^{1D}, and R^{1E} is selected, independently, from H, halogen, optionally substituted C1-C6 alkyl, and optionally substituted C1-C6 alkoxy;

R^{1B} is selected from optionally substituted C1-C6 alkyl or optionally substituted C1-C6 alkoxy;

R^{1C} is selected from H or halogen;

X² is N or CR^{Z4};

R^{Z4} is selected, independently, from H, halogen, optionally substituted C1-C6 alkyl, and optionally substituted C1-C6 alkoxy;

each of R^{Z1}, R^{Z2}, and R^{Z3} is selected, independently, from H or Ar¹, where one and only one of R^{Z1}, R^{Z2}, and R^{Z3} is Ar¹;

Ar¹ is

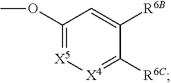

X⁴ is N or CR^{6D};

X⁵ is N or CR^{6E};

R^{6B}, R^{6D}, and R^{6E} are selected, independently, from H, halogen, optionally substituted C1-C6 alkyl, and optionally substituted C1-C6 alkoxy;

R^{6C} is selected from H or halogen; and

where no more than one of X² and X³ is N.

In some embodiments, where when o is 0, R^{Z1} and R^{Z2} are both CH₃, L is —CONH—, R^{1A}, R^{1D}, and R^{1E} are all H, R^{1B} is CF₃, R^{1B} is H, X¹ is N, and R^{Z1} and R^{Z2} are both H, Ar¹ is not O-(3-CF₃-4-FC₆H₃), O-(3-Cl-4-FC₆H₃), O-(6-CF₃-pyrid-3-yl), or O-(p-FC₆H₄); and

where when o is 0, 1, or 2, R^{Z1} and R^{Z2} are both CH₃, L is —CONH—, R^{1A} and R^{1E} are both H, R^{1B} is CF₃, R^{1C} is H, R^{1D} is H or F, X¹ is CH, and R^{Z1} and R^{Z2} are both H, Ar¹ is not O-(p-ClC₆H₄), OC₆H₅, or O-(p-FC₆H₄).

In other embodiments, o is 0 or 1, and/or R^{4A}, R^{4D}, and R^{4E} are each H.

In still other embodiments, the compound, or a pharmaceutically acceptable salt, solvate, or prodrug thereof, or a stereoisomer thereof, has a structure according to the following formula,

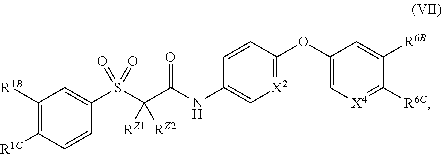

where

R^{Z1} and R^{Z2} are each, independently, unsubstituted C1-C3 alkyl;

X² is CH or N;

X⁴ is CH or N;

R^{1B} is C1 haloalkyl or C1 haloalkoxy;

R^{1C} is H, C1, or F; and

each of R^{6B} and R^{6C} is, independently, H, substituted Cl alkyl, or halogen.

In some embodiments, X² and X⁴ are both CH, or X² and X⁴ are both N, or X² and X⁵ are both N.

In other embodiments, X² is N and X⁴ is CH, or X² is CH and X⁴ is N.

In certain embodiments, R^{1C} is H and R^{1B} is CF₃ or OCF₃.

In particularly embodiments, at least one of R^{6B} and R^{6C} is CF₃, F, or C1.

In some embodiments, R^{Z1} and R^{Z2} are both unsubstituted C1-C3 alkyl, preferably R¹ and R² are both methyl.

In a third aspect, the invention features a compound having a structure according to the following formula,

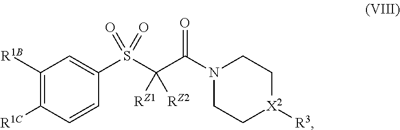

or a pharmaceutically acceptable salt, solvate, or prodrug thereof, or a stereoisomer thereof, where

each of R^{Z1} and R^{Z2} is selected, independently, from optionally substituted C1-C6 alkyl;

X² is CH or N;

R³ is optionally substituted aryl or optionally substituted heteroaryl; and

each of R^{1B} and R^{1C} is, independently, H, halogen, optionally substituted C1-C6 alkyl, or optionally substituted C1-C6 alkoxy.

In some embodiments, X² is N, and R³ is phenyl substituted by CF₃ or halo, or X² is CH, and R³ is phenyl substituted by CF₃ or halo.

In a fourth aspect, the invention features a compound having a structure according to the following formula,

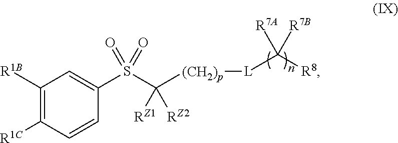

or a pharmaceutically acceptable salt, solvate, or prodrug thereof, or a stereoisomer thereof, where

n is an integer between 0-6, where n is not 0 when R⁸ is H or CF₃;

p is 0, 1, or 2;

L is —C(O)NR^{2A}- or —NR^{2A}C(O)—;

each of R^{Z1} and R^{Z2} is selected, independently, from optionally substituted C1-C6 alkyl; R^{2A} is H or optionally substituted C1-C6 alkyl, or R^{2A} combines with R⁸ to form a heterocyclyl;

each of R^{1B} and R^{1C} is, independently, H, halogen, optionally substituted C1-C6 alkyl, or optionally substituted C1-C6 alkoxy;

each of R^{7A} and R^{7B} is, independently, H, OH, or optionally substituted C1-C6 alkyl;

R⁸ is H, CF₃, optionally substituted aryl, optionally substituted heteroaryl, optionally substituted arylsulfonyl, optionally substituted cycloalkyl, or optionally substituted heterocyclyl; where the optionally substituted groups are substituted with 1, 2, 3, 4, or 5 groups selected from halogen, OH, optionally substituted amino, optionally substituted C1-C6 alkyl, optionally substituted C1-C6 alkoxy, optionally substituted cycloalkyl, optionally substituted heterocyclyl, and —SO₂R⁹;

R⁹ is optionally substituted C1-C6 alkyl, optionally substituted aryl, or optionally substituted heterocyclyl.

In some embodiments, when p is 0, n is 0, 1, or 2, R^{Z1} and R^{Z2} are both CH₃, L is —CONH— or —CONMe-, R^{1B} is CF₃, R^{1C} is H, and R^{7A} and R^{7B} are both H, R⁸ is not any of the following groups:
(a) a phenyl group that is substituted with 1 or 2 substituents selected from F, Cl, CF₃, or O^{t}Bu;
(b) a benzothiazole group substituted with one chloro group; or
(c) a benzimidazole group substituted with one CF₃ group;

and/or

when p is 1, n is 0, R^{Z1} and R^{Z2} are both CH₃, L is —NHCO—, R^{1B} is CF₃, R^{1C} is H, and R^{7A} and R^{7B} are both H, R⁸ is not any of the following groups:
(d) a phenyl group that substituted with 1 or 2 substituents selected from F, C1, CF₃, SO₂Me, SO₂ⁱPr, or unsubstituted oxopyrrolidinyl, or a phenyl group that is substituted with two methyl groups and one methoxy group;
(e) a benzimidazole group substituted with one CF₃ or F group;
(f) an imidazol[1,2-a]pyridine group substituted with one CF₃ group;
(g) a pyridyl group substituted with one group selected from CF₃, CH₃, NHCO^{t}Bu, tert-butyl, and OCH₂CF₃, or a pyridyl group substituted with both a CF₃ group and a SO₂CH₃ group;

and/or

when p is 2, n is 0, R^{Z1} and R^{Z2} are both CH₃, L is —CONH—, —NHCO—, or —NMeCO—, R^{1B} is CF₃, R^{1C} is H, and R^{7A} and R^{7B} are both H, R⁸ is not any of the following groups:
(h) a phenyl group that substituted with 1 or 2 substituents selected from F, Cl, CH₃, CF₃, OMe, SO₂Me, or SO₂ⁱPr;
(i) a pyrimidine group substituted with one CF₃ group, or substituted by both a methyl group and OⁱPr group;
(j) an imidazol[1,2-a]pyridine group substituted with one CF₃ group;
(k) a pyridyl group substituted with one CF₃, CH₃, tert-butyl, OCH₂CF₃, or pivalamido group, or a pyridyl group substituted with both a CF₃ group and a SO₂CH₃ or SO₂ⁱPr group, or both a Cl and OMe group; or
(l) a pyrazole group substituted by one CF₃ group, or by both one CF₃ and one CH₃ group.

In some embodiments, R^{1C} is H and R^{1B} is CF₃ or OCF₃.

In still other embodiments, R^{2A} is H or CH₃.

In certain embodiments, n is 2 and R⁸ is substituted aryl.

In some embodiments, n is 1 and R⁸ is phenyl including a substituent group having the structure —SO₂ (optionally substituted phenyl).

Exemplary compounds encompassed by Formulas (I)-(IX) described herein include Compounds (1)-(227) of Tables 4 and 5, or a pharmaceutically acceptable salt, solvate, or prodrug thereof, or a stereoisomer thereof.

The invention also features the pharmaceutically acceptable salt, solvate, or prodrug thereof, or a stereoisomer thereof, of any of the compounds described herein (e.g., a compound having a structure according to any of Formulas (I)-(IX) such as Compounds (1)-(227) of Tables 4 and 5, preferably Compound (86) of Table 4 and/or Compound (223) of Table 5).

In another aspect, the invention also features a pharmaceutical composition that includes (i) any of the compounds described herein (e.g., a compound having a structure according to any of Formulas (I)-(IX) such as Compounds (1)-(227) of Tables 4 and 5, preferably Compound (86) of Table 4 and/or Compound (223) of Table 5), or a pharmaceutically acceptable salt, solvate, or prodrug thereof, or a stereoisomer thereof, or a conjugate thereof; and (ii) a pharmaceutically acceptable carrier or excipient. In some embodiments, the pharmaceutical composition includes the pharmaceutically acceptable salt of any of the compounds described herein.

In some embodiments, the pharmaceutical composition is formulated in unit dosage form (e.g., a tablet, caplet, capsule, lozenge, film, strip, gelcap, or syrup).

In another aspect, the invention features method to treat a condition modulated by calcium channel activity, the method including administering to a subject in need of such treatment an effective amount of any of the compounds described herein (e.g., a compound having a structure according to any of Formulas (I)-(IX) such as Compounds (1)-(227) of Tables 4 and 5, preferably Compound (86) of Table 4 and/or Compound (223) of Table 5).

In some embodiments, the calcium channel is a T-type calcium channel (e.g., the Ca_{V} 3.1, Ca_{V} 3.2, or Ca_{V} 3.3 channel).

In other embodiments, the calcium channel is an N-type calcium channel (e.g., the Ca_{V} 2.2 channel).

In some embodiments, condition is pain (e.g., inflammatory pain; neuropathic pain; chronic pain, including peripheral neuropathic pain; central neuropathic pain, musculoskeletal pain, headache (e.g., migraine, visceral pain, or mixed pain; or acute pain such as nociceptive pain or post-operative pain), epilepsy, Parkinson's disease, depression, psychosis (e.g., schizophrenia), or tinnitus.

In some embodiments, the peripheral neuropathic pain is post-herpetic neuralgia, diabetic neuropathic pain, neuropathic cancer pain, failed back-surgery syndrome, trigeminal neuralgia, or phantom limb pain;

the central neuropathic pain is multiple sclerosis related pain, Parkinson disease related pain, post-stroke pain, post-traumatic spinal cord injury pain, or pain in dementia;

the musculoskeletal pain is osteoarthritic pain and fibromyalgia syndrome; inflammatory pain such as rheumatoid arthritis, or endometriosis;

the headache is migraine, cluster headache, tension headache syndrome, facial pain, or headache caused by other diseases;

the visceral pain is interstitial cystitis, irritable bowel syndrome, or chronic pelvic pain syndrome; or

the mixed pain is lower back pain, neck and shoulder pain, burning mouth syndrome, or complex regional pain syndrome.

As used herein, the term “alkyl,” “alkenyl” and “alkynyl” include straight-chain, branched-chain and cyclic monovalent substituents, as well as combinations of these, containing only C and hours when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-10 carbons (e.g., C1-C10 alkyl) or 2-10 carbons (e.g., C2-C10 alkenyl or C2-C10 alkynyl). In some embodiments, the alkyl groups are C1-C8, C1-C6, C1-C4, C1-C3, or C1-C2 alkyl groups; or C2-C8, C2-C6, C2-C4, or C2-C3 alkenyl or alkynyl groups. Further, any hydrogen atom on one of these groups can be replaced with a halogen atom, and in particular a fluoro or chloro, and still be within the scope of the definition of alkyl, alkenyl and alkynyl. For example, CF₃ is a C1 alkyl. These groups may be also be substituted by other substituents as described herein.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In some embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In some embodiments, the heteroatom is O or N.

The designated number of carbons in heteroforms of alkyl, alkenyl and alkynyl includes the heteroatom count. For example, if heteroalkyl is defined as C1-C6, it will contain 1-6 C, N, O, or S atoms such that the heteroalkyl contains at least one C atom and at least one heteroatom, for example 1-5 carbons and 1 N atom, or 1-4 carbons and 2 N atoms. Similarly, when heteroalkyl is defined as C1-C6 or C1-C4, it would contain 1-5 carbons or 1-3 carbons respectively, i.e., at least one C is replaced by O, N or S. Accordingly, when heteroalkenyl or heteroalkynyl is defined as C2-C6 (or C2-C4), it would contain 2-6 or 2-4 C, N, O, or S atoms, since the heteroalkenyl or heteroalkynyl contains at least one carbon atom and at least one heteroatom, e.g. 2-5 carbons and 1 N atom, or 2-4 carbons, and 2 O atoms. Further, heteroalkyl, heteroalkenyl or heteroalkynyl substituents may also contain one or more carbonyl groups. Examples of heteroalkyl, heteroalkenyl and heteroalkynyl groups include CH₂OCH₃, CH₂N(CH₃)₂, CH₂OH, (CH₂)ₙNR₂, OR, COOR, CONR₂, (CH₂)ₙ OR, (CH₂)ₙCOR, (CH₂)ₙCOOR, (CH₂)ₙSR, (CH₂)ₙSOR, (CH₂)ₙSO₂R, (CH₂)ₙCONR₂, NRCOR, NRCOOR, OCONR₂, OCOR and the like wherein the R group contains at least one C and the size of the substituent is consistent with the definition of e.g., alkyl, alkenyl, and alkynyl, as described herein.

As used herein, the terms “alkylene,” “alkenylene” and “alkynylene” refer to divalent or trivalent groups having a specified size, typically C1-C2, C1-C3, C1-C4, C1-C6, or C1-C8 for the saturated groups (e.g., alkylene) and C2-C3, C2-C4, C2-C6, or C2-C8 for the unsaturated groups (e.g., alkenylene or alkynylene). They include straight-chain, branched-chain and cyclic forms as well as combinations of these, containing only C and H when unsubstituted. Because they are divalent, they can link together two parts of a molecule, as exemplified by X in the compounds described herein. Examples are methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a Cl alkylene that is substituted by ═O, for example.

Heteroalkylene, heteroalkenylene and heteroalkynylene are similarly defined as divalent groups having a specified size, typically C1-C3, C1-C4, C1-C6, or C1-C8 for the saturated groups and C2-C3, C2-C4, C2-C6, or C2-C8 for the unsaturated groups. They include straight chain, branched chain and cyclic groups as well as combinations of these, and they further contain at least one carbon atom but also contain one or more O, S or N heteroatoms or combinations thereof within the backbone residue, whereby each heteroatom in the heteroalkylene, heteroalkenylene or heteroalkynylene group replaces one carbon atom of the alkylene, alkenylene or alkynylene group to which the heteroform corresponds. As is understood in the art, these heteroforms do not contain more than three contiguous heteroatoms.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, pyridyl, indolyl, pyrimidyl, pyridazinyl, benzothiazolyl or benzimidazolyl, pyrazolyl, imidazolyl, isoxazolyl, thiazolyl, benzothiazolyl, indolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of C1-C8, C1-C6, or more particularly C1-C4 or C1-C3 when saturated or C2-C8, C2-C6, C2-C4, or C2-C3 when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(C6-C12)alkyl(C1-C8), aryl(C6-C12)alkenyl(C2-C8), or aryl(C6-C12)alkynyl(C2-C8), plus the heteroforms. A typical example is phenylmethyl, commonly referred to as benzyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′,NR′C(O)OR′, NR′C(O)NR′₂, NR′SO₂NR′₂, or NR′SO₂R′, wherein each R′ is independently hours or an optionally substituted group selected from alkyl, alkenyl, alkynyl, heteroalkyl, heteroalkenyl, heteroalkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Optional substituents on a non-aromatic group (e.g., alkyl, alkenyl, and alkynyl groups), are typically selected from the same list of substituents suitable for aromatic or heteroaromatic groups and may further be selected from ═O and ═NOR′ where R′ is hours or an optionally substituted group selected from alkyl, alkenyl, alkynyl, heteroalkyl, heteroalkenyl, heteroalkynyl, heteroaryl, and aryl (all as defined above).

Halo may be any halogen atom, especially F, Cl, Br, or I, and more particularly it is fluoro or chloro.

In general, a substituent group (e.g., alkyl, alkenyl, alkynyl, or aryl (including all heteroforms defined above)) may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included. For example, where a group is substituted, the group may be substituted with 1, 2, 3, 4, 5, or 6 substituents. Optional substituents include, but are not limited to: C1-C6 alkyl or heteroaryl, C2-C6 alkenyl or heteroalkenyl, C2-C6 alkynyl or heteroalkynyl, halogen; aryl, heteroaryl, azido(—N₃), nitro (—NO₂), cyano (—CN), acyloxy(—OC(═O)R′), acyl (—C(C═O)R′), alkoxy (—OR′), amido (—NR′C(═O)R″ or —C(═O)NRR′), amino (—NRR′), carboxylic acid (—CO₂H), carboxylic ester (—CO₂R′), carbamoyl (—OC(═O)NR′R″ or —NRC(═O)OR′), hydroxy (—OH), isocyano (—NC), sulfonate (—S(═O)₂OR), sulfonamide (—S(═O)₂NRR′ or —NRS(═O)₂R′), or sulfonyl (—S(═O)₂R), where each R or R′ is selected, independently, from H, C1-C6 alkyl or heteroaryl, C2-C6 alkenyl or heteroalkenyl, 2C-6C alkynyl or heteroalkynyl, aryl, or heteroaryl. A substituted group may have, for example, 1, 2, 3, 4, 5, 6, 7, 8, or 9 substituents.

The term an “effective amount” of an agent (e.g., a compound according to any of Formulas (I)-(IX) or compounds (1)-(227) of Tables 4 and 5), as used herein, is that amount sufficient to effect beneficial or desired results, such as clinical results, and, as such, an “effective amount” depends upon the context in which it is being applied. For example, in the context of administering an agent that is a modulator of a calcium channel (e.g., N- and/or T-type channels), an effective amount of an agent is, for example, an amount sufficient to achieve a change in calcium channel activity as compared to the response obtained without administration of the agent.

The term “pharmaceutical composition,” as used herein, represents a composition containing a compound described herein (e.g., a compound according to any of Formulas (I)-(IX) or compounds (1)-(227) of Tables 4 and 5), formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, represents those prodrugs of the compounds of the present invention that are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, represents those salts of the compounds described here (e.g., a compound according to any of Formulas (I)-(IX) or compounds (1)-(227) of Tables 4 and 5) that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention (e.g., a compound according to any of Formulas (I)-(IX) or compounds (1)-(227) of Tables 4 and 5) may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “pharmaceutically acceptable solvate” as used herein means a compound as described herein (e.g., a compound according to any of Formulas (I)-(IX) or compounds (1)-(227) of Tables 4 and 5) where molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the molecule is referred to as a “hydrate.”

The term “prevent,” as used herein, refers to prophylactic treatment or treatment that prevents one or more symptoms or conditions of a disease, disorder, or conditions described herein (for example, pain (e.g., chronic or acute pain), epilepsy, Alzheimer's disease,

Parkinson's disease, cardiovascular disease, diabetes, cancer, sleep disorders, obesity, psychosis such as schizophrenia, overactive bladder, renal disease, neuroprotection, addiction, and male birth control). Preventative treatment can be initiated, for example, prior to (“pre-exposure prophylaxis”) or following (“post-exposure prophylaxis”) an event that precedes the onset of the disease, disorder, or conditions. Preventive treatment that includes administration of a compound described herein (e.g., a compound according to any of Formulas (I)-(IX) or compounds (1)-(227) of Tables 4 and 5), or a pharmaceutically acceptable salt or solvate thereof, or a pharmaceutical composition thereof, can be acute, short-term, or chronic. The doses administered may be varied during the course of preventative treatment.

The term “prodrug,” as used herein, represents compounds that are rapidly transformed in vivo to the parent compound of the above formula, for example, by hydrolysis in blood. Prodrugs of the compounds described herein (e.g., a compound according to any of Formulas (I)-(IX) or compounds (1)-(227) of Tables 4 and 5) may be conventional esters. Some common esters that have been utilized as prodrugs are phenyl esters, aliphatic (C1-C8 or C8-C24) esters, cholesterol esters, acyloxymethyl esters, carbamates, and amino acid esters. For example, a compound that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference. Preferably, prodrugs of the compounds of the present invention are suitable for use in contact with the tissues of humans and animals with undue toxicity, irritation, allergic response, and the like, commensurate with a reasonable benefit/risk ratio, and effective for their intended use.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to compounds (e.g., a compound according to any of Formulas (I)-(IX) or compounds (1)-(227) of Tables 4 and 5) when modified so as to be included in a conjugate of this type.

As used herein, and as well understood in the art, “to treat” a condition or “treatment” of the condition (e.g., the conditions described herein such as pain (e.g., chronic or acute pain), epilepsy, Alzheimer's disease, Parkinson's disease, cardiovascular disease, diabetes, cancer, sleep disorders, obesity, psychosis such as schizophrenia, overactive bladder, renal disease, neuroprotection, addiction, and male birth control) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “unit dosage form” refers to a physically discrete unit suitable as a unitary dosage for human subjects and other mammals, each unit containing a predetermined quantity of active material (e.g., a compound according to any of Formulas (I)-(IX) or compounds (1)-(227) of Tables 4 and 5) calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gelcap, and syrup.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed.

Other features and advantages of the invention will be apparent from the following Detailed Description and the claims.

### DETAILED DESCRIPTION

### Compounds

The invention features compounds that can inhibit voltage-gated calcium channels (e.g., N- and/or T-type). For example, diarylsulfone compounds can inhibit N-type voltage gated Ca²⁺ channels, and dialkylarylsulfone compounds can inhibit voltage gated N- and T-type calcium channels.

Exemplary compounds are described by any of Formulas (I)-(IX), which include compounds (1)-(227) of Tables 4 and 5. Other embodiments, exemplary methods of synthesis, and uses of these compounds are also described herein.

### Utility and Administration

The compounds described herein are useful in the methods of the invention and, while not bound by theory, are believed to exert their desirable effects through their ability to modulate the activity of calcium channels, particularly the activity of N-type calcium channels. This makes them useful for treatment of certain conditions where modulation of N-type calcium channels is desired, including pain, epilepsy, migraine, Parkinson's disease, depression, schizophrenia, psychosis, and tinnitus.

Modulation of Calcium Channels

The entry of calcium into cells through voltage-gated calcium channels mediates a wide variety of cellular and physiological responses, including excitation-contraction coupling, hormone secretion and gene expression (e.g., Miller et al., Science 235:46-52 (1987); Augustine et al., Annu Rev Neurosci 10: 633-693 (1987)). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter, which also affects neurite outgrowth and growth cone migration in developing neurons.

Calcium channels mediate a variety of normal physiological functions, and are also implicated in a number of human disorders as described herein. For example, calcium channels also have been shown to mediate the development and maintenance of the neuronal sensitization and hyperexcitability processes associated with neuropathic pain, and provide attractive targets for the development of analgesic drugs (reviewed in Vanegas et al., Pain 85: 9-18 (2000)). Native calcium channels have been classified by their electrophysiological and pharmacological properties into T-, L-, N-, P/Q- and R-types (reviewed in Catterall, Annu Rev Cell Dev Biol 16: 521-555, 2000; Huguenard, Annu Rev Physiol 58: 329-348, 1996). The L-, N- and P/Q-type channels activate at more positive potentials (high voltage-activated) and display diverse kinetics and voltage-dependent properties (Id.).

The modulation of ion channels by the compounds described herein (e.g., a compound according to any of Formulas (I)-(IX) or compounds (1)-(227) of Tables 4 and 5) can be measured according to methods known in the art (e.g., in the references provided herein). Modulators of ion channels, e.g., voltage gated calcium ion channels, and the medicinal chemistry or methods by which such compounds can be identified, are also described in, for example: Birch et al., Drug Discovery Today, 9(9):410-418 (2004); Audesirk, “Chapter 6-Electrophysiological Analysis of Ion Channel Function,” Neurotoxicology: Approaches and Methods, 137-156 (1995); Camerino et al., “Chapter 4: Therapeutic Approaches to Ion Channel Diseases,” Advances in Genetics, 64:81-145 (2008); Petkov, “Chapter 16-Ion Channels,” Pharmacology: Principles and Practice, 387-427 (2009); Standen et al., “Chapter 15-Patch Clamping Methods and Analysis of Ion Channels,” Principles of Medical Biology, Vol. 7, Part 2, 355-375 (1997); Xu et al., Drug Discovery Today, 6(24):1278-1287 (2001); and Sullivan et al., Methods Mol. Biol. 114:125-133 (1999). Exemplary experimental methods are also provided in the Examples.

N-Type Calcium Channels

Mutations in calcium channel α1 subunit genes in animals can provide important clues to potential therapeutic targets for pain intervention. Genetically altered mice null for the α1B N-type calcium channel gene have been reported by several independent groups (Ino et al., Proc. Natl. Acad. Sci. USA 98:5323-5328 (2001); Kim et al., Mol Cell Neurosci 18:235-245 (2001); Kim et al., Neuron 31:35-45 (2001); Saegusa et al., Proc. Natl. Acad. Sci. USA 97:6132-6137 (2000); and Hatakeyama et al., NeuroReport 12:2423-2427 (2001)). These studies indicate that the N-type channel may be a potential target for mood disorders as well as pain.

In a variety of animal models, the selective block of N-type channels via intrathecal administration of ziconotide significantly depresses the formalin phase 2 response, thermal hyperalgesia, mechanical allodynia and post-surgical pain (e.g., Malmberg et al., J Neurosci 14: 4882-4890 (1994); Bowersox et al., J Pharmacol Exp Ther 279: 1243-1249 (1996); Sluka, J Pharmacol Exp Ther 287:232-237 (1998); and Wang et al., Soc Neurosci Abstr 24: 1626 (1998)).

Gabapentin (1-(aminomethyl)cyclohexaneacetic acid (Neurontin®)), is an anticonvulsant that also acts on N-type channels. Though not specific for N-type calcium channels, subsequent work has demonstrated that gabapentin is also successful at preventing hyperalgesia in a number of different animal pain models, including chronic constriction injury (CCI), heat hyperalgesia, inflammation, diabetic neuropathy, static and dynamic mechanical allodynia associated with postoperative pain (e.g., Cesena et al., Neurosci Lett 262: 101-104 (1999); Field et al., Pain 80: 391-398 (1999); Cheng et al., Anesthesiology 92: 1126-1131 (2000); and Nicholson, Acta Neurol Scand 101: 359-371 (2000)).

T-Type Calcium Channels

T-type channels can be distinguished by having a more negative range of activation and inactivation, rapid inactivation, slow deactivation, and smaller single-channel conductances. There are three subtypes of T-type calcium channels that have been molecularly, pharmacologically, and elecrophysiologically identified: these subtypes have been termed a 1 G, α1H, and α1I (alternately called Ca_{V} 3.1, Ca_{V} 3.2 and Ca_{V} 3.3 respectively).

T-type calcium channels are involved in various medical conditions. In mice lacking the gene expressing the 3.1 subunit, resistance to absence seizures was observed (Kim et al., Mol. Cell Neurosci. 18(2): 235-245 (2001)). Other studies have also implicated the 3.2 subunit in the development of epilepsy (Su et al., J. Neurosci. 22: 3645-3655 (2002)). There is also evidence that some existing anticonvulsant drugs, such as ethosuximide, function through the blockade of T-type channels (Gomora et al., Mol. Pharmacol. 60: 1121-1132 (2001)).

Low voltage-activated calcium channels are highly expressed in tissues of the cardiovascular system. There is also a growing body of evidence that suggests that T-type calcium channels are abnormally expressed in cancerous cells and that blockade of these channels may reduce cell proliferation in addition to inducing apoptosis. Recent studies also show that the expression of T-type calcium channels in breast cancer cells is proliferation state dependent, i.e. the channels are expressed at higher levels during the fast-replication period, and once the cells are in a non-proliferation state, expression of this channel is minimal. Therefore, selectively blocking calcium channel entry into cancerous cells may be a valuable approach for preventing tumor growth (e.g., PCT Patent Publication Nos. WO 05/086971 and WO 05/77082; Taylor et al., World J. Gastroenterol. 14(32): 4984-4991 (2008); Heo et al., Biorganic & Medicinal Chemistry Letters 18:3899-3901 (2008)).

T-type calcium channels may also be involved in still other conditions. A recent study also has shown that T-type calcium channel antagonists inhibit high-fat diet-induced weight gain in mice. In addition, administration of a selective T-type channel antagonist reduced body weight and fat mass while concurrently increasing lean muscle mass (e.g., Uebele et al., The Journal of Clinical Investigation, 119(6):1659-1667 (2009)). T-type calcium channels may also be involved in pain (see for example: US Patent Publication No. 2003/0086980; PCT Publication Nos. WO 03/007953 and WO 04/000311). In addition to cardiovascular disease, epilepsy (see also US Patent Publication No. 2006/0025397), cancer, and chronic or acute pain, T-type calcium channels have been implicated in diabetes (US Patent Publication No. 2003/0125269), sleep disorders (US Patent Publication No. 2006/0003985), Parkinson's disease and psychosis such as schizophrenia (US Patent Publication No. 2003/0087799); overactive bladder (Sui et al., British Journal of Urology International 99(2): 436-441 (2007); US Patent Publication No. 2004/0197825), renal disease (Hayashi et al., Journal of Pharmacological Sciences 99: 221-227 (2005)), anxiety and alcoholism (US Patent Publication No. 2009/0126031), neuroprotection, and male birth control.

Diseases and Conditions

Exemplary conditions that can be treated using the compounds described herein include pain (e.g., chronic or acute pain), epilepsy, Alzheimer's disease, Parkinson's disease, diabetes; cancer; sleep disorders; obesity; psychosis such as schizophrenia; overactive bladder; renal disease, neuroprotection, and addiction. For example, the condition can be pain (e.g., neuropathic pain or post-surgery pain), epilepsy, migraine, Parkinson's disease, depression, schizophrenia, psychosis, or tinnitus.

Epilepsy as used herein includes but is not limited to partial seizures such as temporal lobe epilepsy, absence seizures, generalized seizures, and tonic/clonic seizures.

Cancer as used herein includes but is not limited to breast carcinoma, neuroblastoma, retinoblastoma, glioma, prostate carcinoma, esophageal carcinoma, fibrosarcoma, colorectal carcinoma, pheochromocytoma, adrenocarcinoma, insulinoma, lung carcinoma, melanoma, and ovarian cancer.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

In treating osteoarthritic pain, joint mobility can also improve as the underlying chronic pain is reduced. Thus, use of compounds of the present invention to treat osteoarthritic pain inherently includes use of such compounds to improve joint mobility in patients suffering from osteoarthritis.

The compounds described herein can be tested for efficacy in any standard animal model of pain. Various models test the sensitivity of normal animals to intense or noxious stimuli (physiological or nociceptive pain). These tests include responses to thermal, mechanical, or chemical stimuli. Thermal stimuli usually involve the application of hot stimuli (typically varying between 42-55° C.) including, for example: radiant heat to the tail (the tail flick test), radiant heat to the plantar surface of the hindpaw (the Hargreaves test), the hotplate test, and immersion of the hindpaw or tail into hot water. Immersion in cold water, acetone evaporation, or cold plate tests may also be used to test cold pain responsiveness. Tests involving mechanical stimuli typically measure the threshold for eliciting a withdrawal reflex of the hindpaw to graded strength monofilament von Frey hairs or to a sustained pressure stimulus to a paw (e.g., the Ugo Basile analgesiometer). The duration of a response to a standard pinprick may also be measured. When using a chemical stimulus, the response to the application or injection of a chemical irritant (e.g., capsaicin, mustard oil, bradykinin, ATP, formalin, acetic acid) to the skin, muscle joints or internal organs (e.g., bladder or peritoneum) is measured.

In addition, various tests assess pain sensitization by measuring changes in the excitability of the peripheral or central components of the pain neural pathway. In this regard, peripheral sensitization (i.e., changes in the threshold and responsiveness of high threshold nociceptors) can be induced by repeated heat stimuli as well as the application or injection of sensitizing chemicals (e.g., prostaglandins, bradykinin, histamine, serotonin, capsaicin, or mustard oil). Central sensitization (i.e., changes in the excitability of neurons in the central nervous system induced by activity in peripheral pain fibers) can be induced by noxious stimuli (e.g., heat), chemical stimuli (e.g., injection or application of chemical irritants), or electrical activation of sensory fibers.

Various pain tests developed to measure the effect of peripheral inflammation on pain sensitivity can also be used to study the efficacy of the compounds (Stein et al., Pharmacol. Biochem. Behav. (1988) 31: 445-451; Woolf et al., Neurosci. (1994) 62: 327-331). Additionally, various tests assess peripheral neuropathic pain using lesions of the peripheral nervous system. One such example is the “axotomy pain model” (Watson, J. Physiol. (1973) 231:41). Other similar tests include the SNL test which involves the ligation of a spinal segmental nerve (Kim and Chung, Pain (1992) 50: 355), the Seltzer model involving partial nerve injury (Seltzer, Pain (1990) 43: 205-18), the spared nerve injury (SNI) model (Decosterd and Woolf, Pain (2000) 87:149), chronic constriction injury (CCI) model (Bennett (1993) Muscle Nerve 16: 1040), tests involving toxic neuropathies such as diabetes (streptozocin model), pyridoxine neuropathy, taxol, vincristine, and other antineoplastic agent-induced neuropathies, tests involving ischaemia to a nerve, peripheral neuritis models (e.g., CFA applied peri-neurally), models of post-herpetic neuralgia using HSV infection, and compression models.

In all of the above tests, outcome measures may be assessed, for example, according to behavior, electrophysiology, neurochemistry, or imaging techniques to detect changes in neural activity.

Exemplary models for the treatment of pain and epilepsy include, but are not limited to, the following.

### Models of Pain

L5/L6 Spinal Nerve Ligation (SNL)-Chung Pain Model

The Spinal Nerve Ligation is an animal model representing peripheral nerve injury generating a neuropathic pain syndrome. In this model, experimental animals develop the clinical symptoms of tactile allodynia and hyperalgesia. L5/L6 Spinal nerve ligation (SNL) injury can be induced using the procedure of Kim and Chung (Kim et al., Pain 50:355-363 (1992)) in male Sprague-Dawley rats.

Assessment of Tactile Allodynia—Von Frey

The assessment of tactile allodynia can consist of measuring the withdrawal threshold of the paw ipsilateral to the site of nerve injury in response to probing with a series of calibrated von Frey filaments (innocuous stimuli). Animals can be acclimated to the suspended wire-mesh cages for 30 minutes before testing. Each von Frey filament can be applied perpendicularly to the plantar surface of the ligated paw of rats for 5 seconds. A positive response may be indicated by a sharp withdrawal of the paw. Measurements can be taken before and after administration of test articles. The paw withdrawal threshold can be determined by the non-parametric method of Dixon (Dixon, Ann. Rev. Pharmacol. Toxicol. 20:441-462 (1980)), in which the stimulus was incrementally increased until a positive response was obtained, and then decreased until a negative result was observed. The protocol can be repeated until three changes in behaviour are determined (“up and down” method) (Chaplan et al., J. Neurosci. Methods 53:55-63 (1994)). For example, the 50% paw withdrawal threshold can be determined as (10^{[Zf+kδ]})/10,000, where X_{f}=the value of the last von Frey filament employed, k=Dixon value for the positive/negative pattern, and δ=the logarithmic difference between stimuli. The cut-off values for rats can be no less than 0.2 g and no higher than 15 g (5.18 filament); for mice no less than 0.03 g and no higher than 2.34 g (4.56 filament). A significant drop of the paw withdrawal threshold compared to the pre-treatment baseline is considered tactile allodynia.

Assessment of Thermal Hypersensitivity—Hargreaves

The method of Hargreaves and colleagues (Hargreaves et al., Pain 32:77-8 (1988)) can be employed to assess paw-withdrawal latency to a noxious thermal stimulus. Rats may be allowed to acclimate within a Plexiglas enclosure on a clear glass plate for 30 minutes. A radiant heat source (e.g., halogen bulb coupled to an infrared filter) can then be activated with a timer and focused onto the plantar surface of the affected paw of treated rats. Paw-withdrawal latency can be determined by a photocell that halts both lamp and timer when the paw is withdrawn. The latency to withdrawal of the paw from the radiant heat source can be determined prior to L5/L6 SNL, 7-14 days after L5/L6 SNL but before drug, as well as after drug administration. A maximal cut-off of 33 seconds is typically employed to prevent tissue damage. Paw withdrawal latency can be thus determined to the nearest 0.1 second. A significant drop of the paw withdrawal latency from the baseline indicates the status of thermal hyperalgesia. Antinociception is indicated by a reversal of thermal hyperalgesia to the pre-treatment baseline or a significant (p<0.05) increase in paw withdrawal latency above this baseline. Data can be converted to % anti hyperalgesia or % anti nociception by the formula: (100×(test latency−baseline latency)/(cut-off−baseline latency) where cut-off is 21 seconds for determining anti hyperalgesia and 40 seconds for determining anti nociception.

### Models of Epilepsy

6 Hz Psychomotor Seizure Model of Partial Epilepsy

Compounds can also be evaluated for the protection against seizures induced by a 6 Hz, 0.2 ms rectangular pulse width of 3 s duration, at a stimulus intensity of 32 mA (CC97) applied to the cornea of male CF1 mice (20-30 g) according to procedures described by Barton et al, “Pharmacological Characterization of the 6 Hz Psychomotor Seizure Model of Partial Epilepsy,” Epilepsy Res. 47(3):217-27 (2001). Seizures can be characterized by the expression of one or more of the following behaviours: stun, forelimb clonus, twitching of the vibrissae and Straub-tail immediately following electrical stimulation. Animals can be considered “protected” if following pre-treatment with a compound the 6 Hz stimulus failed to evoke a behavioural response as describe above.

Mouse Rotarod Assay

To assess a compound's undesirable side effects (toxicity), animals can be monitored for overt signs of impaired neurological or muscular function. In mice, the rotarod procedure (Dunham and Miya, J. Am. Pharmacol. Assoc. 46:208-209 (1957)) is used to disclose minimal muscular or neurological impairment (MMI). When a mouse is placed on a rod that rotates at a speed of 6 rpm, the animal can maintain its equilibrium for long periods of time. The animal is considered toxic if it falls off this rotating rod three times during a 1-min period. In addition to MMI, animals may exhibit a circular or zigzag gait, abnormal body posture and spread of the legs, tremors, hyperactivity, lack of exploratory behavior, somnolence, stupor, catalepsy, loss of placing response and changes in muscle tone.

Lamina Assay—Recordings on Lamina I/II Spinal Cord Neurons.

Male Wistar rats (P6 to P9 for voltage-clamp and P15 to P18 for current-clamp recordings) can be anaesthetized through intraperitoneal injection of Inactin (Sigma). The spinal cord can then be rapidly dissected out and placed in an ice-cold solution protective sucrose solution containing (in mM): 50 sucrose, 92 NaCl, 15 D-Glucose, 26 NaHCO₃, 5 KCl, 1.25 NaH₂PO₄, 0.5 CaCl₂, 7 MgSO₄, 1 kynurenic acid, and bubbled with 5% CO₂/95% O₂. The meninges, dura, and dorsal and ventral roots can then removed from the lumbar region of the spinal cord under a dissecting microscope. The “cleaned” lumbar region of the spinal cord may be glued to the vibratome stage and immediately immersed in ice cold, bubbled, sucrose solution. For current-clamp recordings, 300 to 350 μm parasagittal slices can be cut to preserve the dendritic arbour of lamina I neurons, while 350 to 400 μm transverse slices can be prepared for voltage-clamped Na_{V} channel recordings. Slices may be allowed to recover for 1 hour at 35° C. in Ringer solution containing (in mM): 125 NaCl, 20 D-Glucose, 26 NaHCO₃, 3 KCl, 1.25 NaH₂PO₄, 2 CaCl₂, 1 MgCl₂, 1 kynurenic acid, 0.1 picrotoxin, bubbled with 5% CO₂/95% O₂. The slice recovery chamber can then returned to room temperature (20 to 22° C.) for recordings.

Neurons may be visualized using IR-DIC optics (Zeiss Axioskop 2 FS plus, Gottingen, Germany), and neurons from lamina I and the outer layer of lamina II can be selected based on their location relative to the substantia gelatinosa layer. Neurons can be patch-clamped using borosilicate glass patch pipettes with resistances of 3 to 6 MΩ. Current-clamp recordings of lamina I/II neurons in the intact slice, the external recording solution was the above Ringer solution, while the internal patch pipette solution contained (in mM): 140 KGluconate, 4 NaCl, 10 HEPES, 1 EGTA, 0.5 MgCl₂, 4 MgATP, 0.5 Na₂GTP, adjusted to pH 7.2 with 5 M KOH and to 290 mOsm with D-Mannitol (if necessary). Tonic firing neurons can be selected for current-clamp experiments, while phasic, delayed onset and single spike neurons may be discarded (22). Recordings can be digitized at 50 kHz and low-pass filtered at 2.4 kHz.

In addition to being able to modulate a particular calcium channel (e.g., Ca_{V} 2.2, Ca_{V} 3.1, Ca_{V} 3.2, or Ca_{V} 3.3), it may be desirable that the compound has very low activity with respect to the hERG K⁺ channel, which is expressed in the heart: compounds that block this channel with high potency may cause reactions which are fatal. See, e.g., Bowiby et al., “hERG (KCNH2 or K_{V}11.1 K⁺ Channels: Screening for Cardiac Arrhythmia Risk,” Curr. Drug Metab. 9(9):965-70 (2008)). Thus, for a compound that modulates calcium channel activity, it may also be shown that the hERG K⁺ channel is not inhibited or only minimally inhibited as compared to the inhibition of the primary channel targeted. Similarly, it may be desirable that the compound does not inhibit cytochrome p450, an enzyme that is required for drug detoxification. Such compounds may be particularly useful in the methods described herein.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Exemplary assays are also described in the Examples.

### Pharmaceutical Compositions

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, or therapy—the compounds are formulated in ways consonant with these parameters. A summary of such techniques is found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

The compounds described herein (e.g., a compound according to any of Formulas (I)-(IX) or compounds (1)-(227) of Tables 4 and 5) may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, gastrointestinal, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

In general, for use in treatment, the compounds described herein (e.g., a compound according to any of Formulas (I)-(IX) or compounds (1)-(227) of Tables 4 and 5) may be used alone, as mixtures of two or more compounds or in combination with other pharmaceuticals. An example of other pharmaceuticals to combine with the compounds described herein (e.g., a compound according to any of Formulas (I)-(IX) or compounds (1)-(227) of Tables 4 and 5) would include pharmaceuticals for the treatment of the same indication. For example, in the treatment of pain, a compound may be combined with another pain relief treatment such as an NSAID, or a compound which selectively inhibits COX-2, or an opioid, or an adjuvant analgesic such as an antidepressant. Another example of a potential pharmaceutical to combine with the compounds described herein (e.g., a compound according to any of Formulas (I)-(IX) or compounds (1)-(227) of Tables 4 and 5) would include pharmaceuticals for the treatment of different yet associated or related symptoms or indications. Depending on the mode of administration, the compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

The compounds of the invention may be prepared and used as pharmaceutical compositions comprising an effective amount of a compound described herein (e.g., a compound according to any of Formulas (I)-(IX) or compounds (1)-(227) of Tables 4 and 5) and a pharmaceutically acceptable carrier or excipient, as is well known in the art. In some embodiments, the composition includes at least two different pharmaceutically acceptable excipients or carriers.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677, which is herein incorporated by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, and tablets, as is understood in the art.

For administration to animal or human subjects, the dosage of the compounds of the invention may be, for example, 0.01-50 mg/kg (e.g., 0.01-15 mg/kg or 0.1-10 mg/kg). For example, the dosage can be 10-30 mg/kg.

Each compound of a combination therapy, as described herein, may be formulated in a variety of ways that are known in the art. For example, the first and second agents of the combination therapy may be formulated together or separately.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Generally, when administered to a human, the oral dosage of any of the compounds of the combination of the invention will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary. Administration of each drug in a combination therapy, as described herein, can, independently, be one to four times daily for one day to one year, and may even be for the life of the patient. Chronic, long-term administration may be indicated.

### Examples

### Synthesis of the Invention Compounds

The following reaction schemes and examples are intended to illustrate the synthesis of a representative number of compounds. Accordingly, the following examples are intended to illustrate but not to limit the invention. Additional compounds not specifically exemplified may be synthesized using conventional methods in combination with the methods described herein. Exemplary compounds prepared according to methods known in the art and described herein are provided in Tables 4 and 5.

### HiTOPS Purification Protocol

Purification of crude organic mixtures was conducted by a High Throughput Organic Purification (HiTOP) Laboratory using reversed phase preparative HPLC. Two approaches were utilized depending on the nature of the target; a low pH approach (Table 1) or a high pH approach (Table 2). Analytical scale chromatography, as known in the art, was used to determine the type of preparative method required for each sample as well as to conduct final purity checks and product confirmation on collected final material.

TABLE 1
LOW PH METHOD SPECIFIC PREPARATIVE PARAMETERS

| | |
|---|---|
| COLUMN: | Waters Sunfire, C18 OBD, 5 μm 30 × 50 mm |
| | (P/N: 186002570) |

MOBILE PHASE:

| | |
|---|---|
| Solvent A: | HPLC Grade Water w/ 0.1% Formic Acid |
| Solvent B: | HPLC Grade Methanol* w/0.1% Formic Acid |

*HPLC Grade Acetonitrile was used for samples prepared before December 2008

TABLE 2
HIGH PH METHOD SPECIFIC PREPARATIVE PARAMETERS

| COLUMN: | Waters XBridge, C18 OBD, 5 μm, 30 × 50 mm (P/N: |
|---|---|
| | 186002980) |

MOBILE PHASE:

| | |
|---|---|
| Solvent A: | HPLC Grade Water w/ 0.1% Ammonium Hydroxide |
| Solvent B: | HPLC Grade Methanol* w/0.1% Ammonium |
| | Hydroxide |

*HPLC Grade Acetonitrile was used for samples prepared before December 2008

### Preparative Chromatography

Preparative HPLC was performed using the following method specific parameters and the assigned “Narrow” method (Table 3).

TABLE 3
NARROW METHOD PARAMETERS

| INJECTION | Aim to load a maximum of 100 mg of crude | | | | | | | |
|---|---|---|---|---|---|---|---|---|
| VOLUME: | material | | | | | | | |
| COLUMN | Ambient | | | | | | | |
| TEMPERATURE: | | | | | | | | |
| GRADIENT | Gradient of Solvents A and B (as below) | | | | | | | |
| PROFILE: | | | | | | | | |
| | | | Narrow Method | | | | | |
| | Time | Flow | Solvent B (%) | | | | | |
| Step | (min) | (mL/min) | A | B | C | D | E | F |
| 1 | 0.0 | 42.5 | 10 | 15 | 27 | 39 | 51 | 63 |
| 2 | 1.5 | 42.5 | 10 | 15 | 27 | 39 | 51 | 63 |
| 3 | 2.0 | 42.5 | — | 25 | 37 | 49 | 61 | — |
| 4 | 9.5 | 42.5 | 40 | 47 | 59 | 71 | 83 | 73 |
| 5 | 10.5 | 42.5 | 40 | 95 | 95 | 95 | 95 | 95 |
| 6 | 11.5 | 42.5 | 95 | 95 | 95 | 95 | 95 | 95 |

| | |
|---|---|
| TOTAL RUN | 11.5 minutes (Run can be terminated early once |
| TIME: | target is collected) |
| Scan mode: | PDA @ 220 nm and MS Scan from from 220 m/z |
| | to 700 m/z |

### Example 1

### Procedure for the synthesis of 2-methyl-2-((3-(trifluoromethyl)phenyl) sulfonyl)propanoic acid (5)

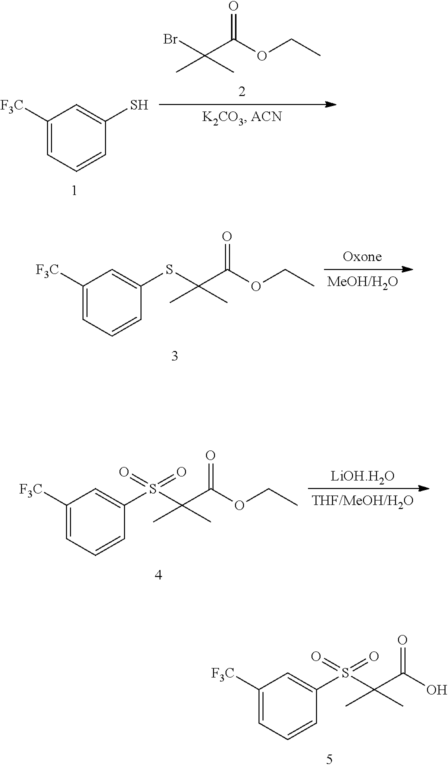

### Preparation of ethyl 2-methyl-2-(3-(trifluoromethyl)phenylthio)propanoate (3)

3-(Trifluoromethyl)benzenethiol (1) (25 g, 140.3 mmol), ethyl 2-bromo-2-methylpropanoate (2) (27.4 g, 140.3 mmol) and K₂CO₃ (24.2 g, 175.4 mmol) were heated at reflux in MeCN (400 mL) for 16 hours. The reaction was cooled, filtered, and concentrated in vacuo. The residue purified by column chromatography (Pet Ether/DCM (80/20)) to give ethyl 2-methyl-2-(3-(trifluoromethyl)phenylthio)propanoate (3) (34.9 g, 85%); ¹H NMR (300 MHz-CD₃Cl) δ 1.49 (s, 6H), 3.65 (s, 3H), 7.45 (t, 1H, J=7.74 Hz), 7.63 (m, 2H), 7.07 (s, 1H).

### Preparation of ethyl 2-methyl-2-(3-(trifluoromethyl)phenylsulfonyl)propanoate (4)

Ethyl 2-methyl-2-(3-(trifluoromethyl)phenylthio)propanoate (3) (34.9 g, 119.4 mmol) and Oxone (220.2 g, 358.2 mmol) were stirred in H₂O/MeOH (330 mL/550 mL) at room temperature for 72 hours. The reaction was filtered, MeOH removed in vacuo, and the aqueous layer extracted with EtOAc. The organics were dried (Na₂SO₄) and concentrated in vacuo to give ethyl 2-methyl-2-(3-(trifluoromethyl)phenylsulfonyl) propanoate (4) (38.4 g, 100%); ¹H NMR (300 MHz-CD₃Cl) δ 1.63 (s, 6H), 3.70 (s, 3H), 7.73 (t, 1H, J=7.86 Hz), 7.95 (d, 1H, J=7.83 Hz), 8.06 (d, 1H, J=7.98 Hz), 8.11 (s, 1H). The product was used without additional purification.

### Preparation of 2-methyl-2-(3-(trifluoromethyl)phenylsulfonyl)propanoic acid (5)

Ethyl 2-methyl-2-(3-(trifluoromethyl)phenylsulfonyl)propanoate (4) (20 g, 61.7 mmol) and LiOH.H₂O (3.9 g, 92.5 mmol) were stirred in THF/MeOH/H₂O (175 mL, 3/1/1) at room temperature for 16 hours. The organics were removed in vacuo, and the aqueous portion acidified to pH 2 with 6M HCl and extracted with EtOAc. The organics were dried (Na₂SO₄) and concentrated in vacuo to give 2-methyl-2-(3-(trifluoromethyl)phenylsulfonyl)propanoic acid (5) (17.1 g, 93%); ¹H NMR (300 MHz-CD₃Cl) δ 1.65 (s, 6H), 7.74 (t, 1H, J=7.71 Hz), 7.95 (d, 1H, J=7.83 Hz), 8.12 (d, 1H, J=8.04 Hz), 8.16 (s, 1H). The product was used without further purification.

### Example 2

### Procedure for the synthesis of 3-(methylsulfonyl)-5-(trifluoromethyl)picolinic acid (7)

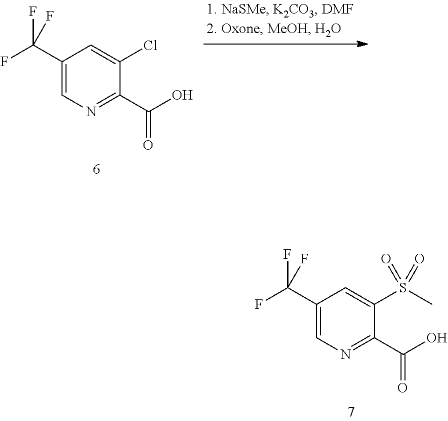 [SMILES: O=C(O)c1ncc(C(F)(F)F)cc1Cl ; CS(=O)(=O)c1cc(C(F)(F)F)cnc1C(=O)O]

3-Chloro-5-(trifluoromethyl)picolinic acid (6) (2.11 g, 10.0 mmol), K₂CO₃ (1.38 g, 10.0 mmol), and NaSMe (1.20 g, 25.0 mmol) were stirred in DMF (15 mL) at 110° C. for 16 h. The reaction was concentrated in vacuo and the residue dissolved in MeOH (80 mL) and H₂O (80 mL). Oxone monopersulfate (30 g, 49 mmol) was added, and the reaction stirred at room temperature for 16 hours. The solid was removed by filtration, and the filtrate basified with 10% NaOH for 30 minutes. The MeOH was removed in vacuo, and the aqueous portion acidified to pH 1 with 6 N HCl, extracted with EtOAc (3×80 mL), dried (Na₂SO₄), and concentrated in vacuo. The residue was recrystallized (with 1 eq. DMF) from EtOAc/hexanes to give 3-(methylsulfonyl)-5-(trifluoromethyl)picolinic acid (7) containing one DMF molecule (1.70 g, 51%); ¹H NMR (300 MHz, CD₃OD) δ 2.88 (s, 3H, DMF), 3.01 (s, 311, DMF), 3.45 (s, 3H), 8.00 (s, 1H, DMF), 8.73 (s, 1H), 9.22 (s, 1H).

### Example 3

### Procedure for the synthesis of 2-(methylsulfonyl)-6-(trifluoromethyl) nicotinic acid (9)

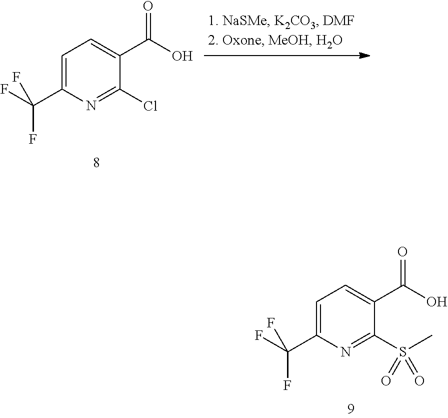 [SMILES: O=C(O)c1ccc(C(F)(F)F)nc1Cl ; CS(=O)(=O)c1nc(C(F)(F)F)ccc1C(=O)O]

3-(Methylsulfonyl)-5-(trifluoromethyl)picolinic acid (9) was prepared in an analogous fashion using 2-chloro-6-(trifluoromethyl)nicotinic acid (8) (5.35 g, 25.3 mmol) to give the required product (5.97 g, 69%) (containing 1 eq. of DMF); ¹H NMR (300 MHz, CD₃OD) δ 2.88 (s, 3H, DMF), 3.01 (s, 3H, DMF), 3.40 (s, 3H), 8.00 (s, 1H, DMF), 8.22 (d, 1H, J=7.5 Hz), 8.49 (d, 1H, J=7.5 Hz).

### Example 4

### Procedure for the synthesis of 2-(isopropylsulfonyl)-6-(trifluoromethyl) nicotinic acid (10)

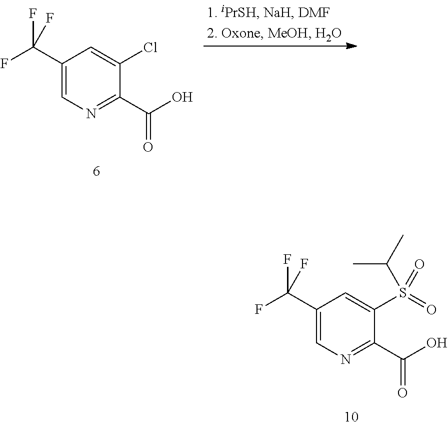 [SMILES: O=C(O)c1ncc(C(F)(F)F)cc1Cl ; CC(C)S(=O)(=O)c1cc(C(F)(F)F)cnc1C(=O)O]

2-(isopropylsulfonyl)-6-(trifluoromethyl)nicotinic acid (10) was prepared in an analogous fashion using 3-chloro-5-(trifluoromethyl)picolinic acid (6) (1.50 g, 7.09 mmol) to give the required (1.4 g, 62%); ¹H NMR (300 MHz, CDCl₃) δ 9.06 (s, 1H), 8.56 (s, 1H), 4.09 (m, 1H), 1.31 (d, 6H, J=6.8 Hz).

### Example 5

### Procedure for the synthesis of 2-(methylsulfonyl)-6-(trifluoromethyl) isonicotinic acid (13)

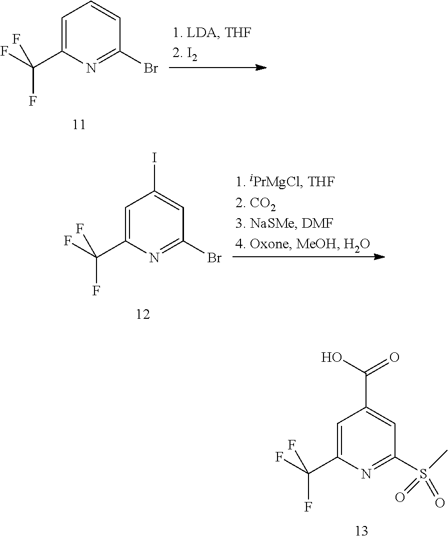 [SMILES: FC(F)(F)c1cccc(Br)n1 ; FC(F)(F)c1cc(I)cc(Br)n1 ; CS(=O)(=O)c1cc(C(=O)O)cc(C(F)(F)F)n1]

### Preparation of 2-bromo-4-iodo-6-(trifluoromethyl)pyridine (12)

Diisopropylamine (2.83 g, 28.0 mmol) was stirred under argon in dry THF (60 mL) at −85° C. nBuLi (1.6 M in hexanes, 17.5 mL, 28 mmol) was added dropwise, and the reaction stirred for 1 hour. 2-Bromo-6-(trifluoromethyl)pyridine (11) (3.00 g, 13.3 mmol) in dry THF (6 mL) was added dropwise, and the reaction stirred for 2 hours. Iodine (I₂; 3.37 g, 13.3 mmol) was added in portions, and the reaction stirred for 30 minutes. The reaction was then quenched with H₂O and extracted with EtOAc (3×30 mL). The organics were dried (Na₂SO₄), concentrated in vacuo, and purified by automated column chromatography (EtOAc/PE, 1:8) to give 2-bromo-4-iodo-6-(trifluoromethyl)pyridine (12) (2.3 g, 49%); ¹H NMR (300 MHz, CDCl₃) δ 7.98 (s, 1H), 8.03 (s, 1H).

### Preparation of 2-(methylsulfonyl)-6-(trifluoromethyl)isonicotinic acid (13)

2-Bromo-4-iodo-6-(trifluoromethyl)pyridine (12) (2.70 g, 7.67 mmol) was stirred under argon in dry THF (30 mL) at −10° C. ⁱPrMgCl (2.0 M, THF, 4.5 mL, 9.0 mmol) was added, and the mixture was stirred at 0° C. for 30 minutes. Carbon dioxide (CO₂) was bubbled through the reaction, and stirring continued for 1.5 hours, allowing to warm to room temperature. The reaction was concentrated in vacuo, taken up in DMF (20 mL), and stirred with NaSMe (0.90 g, 19 mmol) at 100° C. for 2 hours. The reaction was concentrated in vacuo, taken up in MeOH (50 mL) and H₂O (50 mL) with oxone monopersulfate (30 g, 49 mmol), and stirred at room temperature for 3 hours. The reaction was filtered, the filtrate basified with 10% NaOH for 30 minutes, and the MeOH removed in vacuo. The aqueous residue was acidified with 6 N HCl and extracted with EtOAc (3×50 mL). The organics were dried (Na₂SO₄), concentrated in vacuo, and the residue recrystallized from EtOAc/hexanes with the presence of 1 eq. DMF to give 2-(methylsulfonyl)-6-(trifluoromethyl)isonicotinic acid (13) (1.70 g, 51%); ¹H NMR (300 MHz, CD₃OD) δ 2.88 (s, 3H, DMF), 3.01 (s, 3H, DMF), 3.34 (s, 3H), 8.00 (s, 1H, DMF), 8.52 (s, 1H), 8.73 (s, 1H).

### Example 6

### Procedure for the synthesis of (4-((3-(trifluoromethyl)phenyl) sulfonyl)phenyl)methanamine (17)

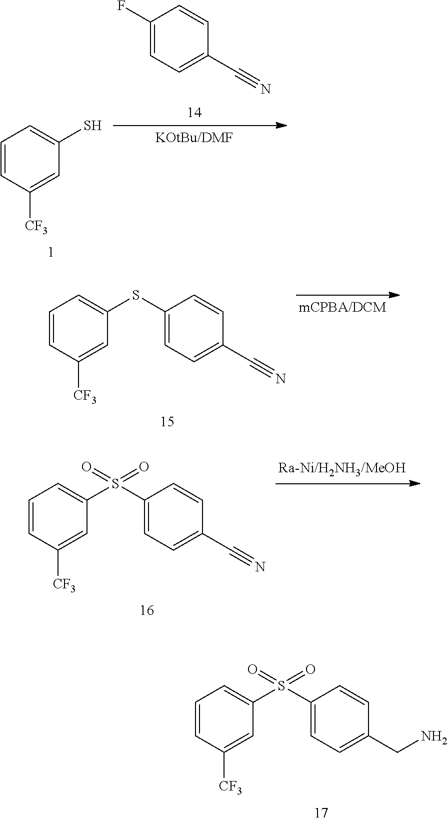 [SMILES: N#Cc1ccc(F)cc1 ; FC(F)(F)c1cccc(S)c1 ; N#Cc1ccc(Sc2cccc(C(F)(F)F)c2)cc1 ; N#Cc1ccc(S(=O)(=O)c2cccc(C(F)(F)F)c2)cc1 ; NCc1ccc(S(=O)(=O)c2cccc(C(F)(F)F)c2)cc1]

### Preparation of 4-((3-(trifluoromethyl)phenyl)thio)benzonitrile (15)

A stream of argon was bubbled through a solution of KO^{t}Bu (3.1 g, 27.8 mmol) in dry DMF (12 mL) at 0° C. for 10 minutes. 3-Trifluoromethyl thiophenol (1) (4.5 g, 25.3 mmol) and 4-fluorobenzonitrile (14) (3.36 g, 27.8 mmol) were added sequentially, and the reaction was heated at 180° C. for 30 minutes in a microwave reactor vessel. The reaction was diluted with EtOAc, washed with H₂O and brine, dried (Na₂SO₄), concentrated in vacuo and the residue purified by automated column chromatography to give 4-((3-(trifluoromethyl)phenyl)thio)benzonitrile (15) (7.06 g, 100%); ¹H NMR (300 MHz, CDCl₃) δ 7.25 (d, 2H, J=8.4 Hz), 7.55 (m, 3H), 7.65 (d, 2H, J=7.92 Hz), 7.75 (s, 1H)

### Preparation of 4-((3-(trifluoromethyl)phenyl)sulfonyl)benzonitrile (16)

4-((3-(trifluoromethyl)phenyl)thio)benzonitrile (15) (7.47 g, 26.7 mmol) and mCPBA (77%, 12.6 g, 56.2 mmol) were stirred in DCM (350 mL) at room temperature for 16 hours. The reaction was washed with 2 M NaOH (2×100 mL), dried (Na₂SO₄), and concentrated in vacuo to give 4-((3-(trifluoromethyl)phenyl)sulfonyl)benzonitrile (16) (8.01 g, 96%); ¹H NMR (300 MHz, CDCl₃) δ 7.72 (t, 1H, J=7.83 Hz), 7.87 (m, 3H), 8.12 (m, 3H), 8.22 (s, 1H). This material was used without further purification.

### Preparation of (4-((3-(trifluoromethyl)phenyl)sulfonyl)phenyl)methanamine (17)

A slurry of Raney nickel was washed twice with MeOH to remove water and provide a enough catalytic material for the reaction. 4-((3-(Trifluoromethyl)phenyl) sulfonyl)benzonitrile (16) (8.01 g, 25.73 mmol) in MeOH (200 mL) was added to the catalyst, and the solution saturated with NH₃ (gas). The reaction was hydrogenated using a Parr apparatus at 55 PSI for 2 hours. The reaction was then filtered, and the filtrate was concentrated in vacuo to give (4-((3-(trifluoromethyl)phenyl)sulfonyl)phenyl) methanamine (17) (7.93 g, 98%). The product was confirmed by positive ion mode LCMS and FIA MS and used without further purification.

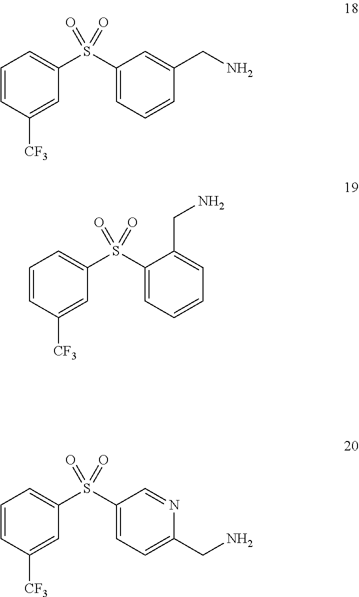 [SMILES: NCc1cccc(S(=O)(=O)c2cccc(C(F)(F)F)c2)c1 ; NCc1ccccc1S(=O)(=O)c1cccc(C(F)(F)F)c1 ; NCc1ccc(S(=O)(=O)c2cccc(C(F)(F)F)c2)cn1]

(2-((3-(trifluoromethyl)phenyl)sulfonyl)phenyl)methanamine (18) and (3-((3-(trifluoromethyl)phenyl)sulfonyl)phenyl)methanamine (19) were prepared in an analogous fashion to (4-((3-(trifluoromethyl)phenyl)sulfonyl)phenyl)methanamine (17) using the appropriately substituted fluorobenzonitrile.

(5-((3-(trifluoromethyl)phenyl)sulfonyl)pyridin-2-yl)methanamine (20) was prepared in an analogous fashion to (4-((3-(trifluoromethyl)phenyl)sulfonyl)phenyl) methanamine (17) using 5-chloropicolinonitrile.

### Example 7

### Procedure for the synthesis of (4-((3-(trifluoromethyl)phenyl)sulfonyl) pyridin-2-yl)methanamine (24)

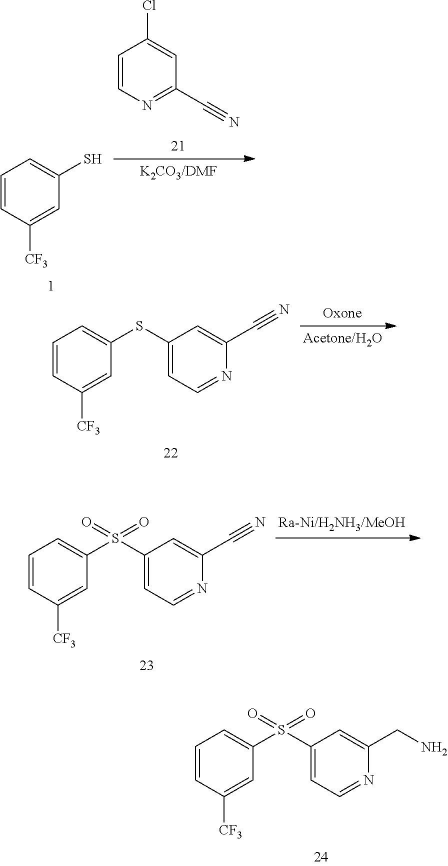 [SMILES: N#Cc1cc(Cl)ccn1 ; FC(F)(F)c1cccc(S)c1 ; N#Cc1cc(Sc2cccc(C(F)(F)F)c2)ccn1 ; N#Cc1cc(S(=O)(=O)c2cccc(C(F)(F)F)c2)ccn1 ; NCc1cc(S(=O)(=O)c2cccc(C(F)(F)F)c2)ccn1]

### Preparation of 4-((3-(trifluoromethyl)phenyl)thio)picolinonitrile (22)

3-Trifluoromethylthiophenol (1) (1.73 g, 9.69 mmol), 4-chloropicolinonitrile (21) (1.22 g, 8.8 mmol), and K₂CO₃ (2.44 g, 12.6 mmol) were heated in DMF (12 mL) at 180° C. for 30 minutes in a microwave reactor. The reaction was diluted with EtOAc, washed with H₂O and brine, dried (Na₂SO₄), concentrated in vacuo, and the residue purified by automated column chromatography (50% EtOAc/Pet ether) to give 4-((3-(trifluoromethyl)phenyl)thio)picolinonitrile (22) (2.34 g, 95%); ¹H NMR (300 MHz, CDCl₃) δ 7.12 (dd, 1H, J=1.56 Hz, 5.34 Hz), 7.27 (d, 1H, J=1.56 Hz), 7.67 (t, 1H, J=7.71 Hz), 7.80 (m, 3H), 8.45 (d, 1H, J=5.34 Hz).

### Preparation of 4-O-(trifluoromethyl)phenyl)sulfonyl)picolinonitrile (23)

4-((3-(Trifluoromethyl)phenyl)thio)picolinonitrile (22) (2.34 g, 8.35 mmol) and oxone (12.83 g, 20.9 mmol) were stirred in acetone/H₂O (130 mL/80 mL) at room temperature for 16 hours. The reaction was concentrated in vacuo, partitioned between DCM and H₂O, the organics separated, dried (Na₂SO₄), and concentrated in vacuo to give (4-((3-(trifluoromethyl)phenyl)sulfonyl)picolinonitrile (23) (2.15 g, 82%); ¹H NMR (300 MHz, CDCl₃) δ 7.79 (t, 1H, J=7.86 Hz), 7.97 (d, 1H, J=7.86 Hz), 8.02 (d, 1H, J=5.01 Hz), 8.14 (s, 1H), 8.19 (d, 1H, J=7.95 Hz), 8.24 (s, 1H), 8.99 (d, 1H, J=4.98 Hz). The product was used without further purification.

### Preparation of (4-((3-(trifluoromethyl)phenyl)sulfonyl)pyridin-2-yl)methanamine (24)

(4-((3-(trifluoromethyl)phenyl)sulfonyl)pyridin-2-yl)methanamine (24) was prepared in an analogous fashion to (4-((3-(trifluoromethyl)phenyl)sulfonyl) phenyl)methanamine (17) using (4-((3-(trifluoromethyl)phenyl)sulfonyl)picolinonitrile (23).

### Example 8

### Procedure for the synthesis of 2-(trifluoromethyl)-6-(4-((3-(trifluoromethyl)phenyl)sulfonyl)benzyl)-6,7-dihydro-5H-pyrrolo[3,4-b]pyridin-5-one (28)

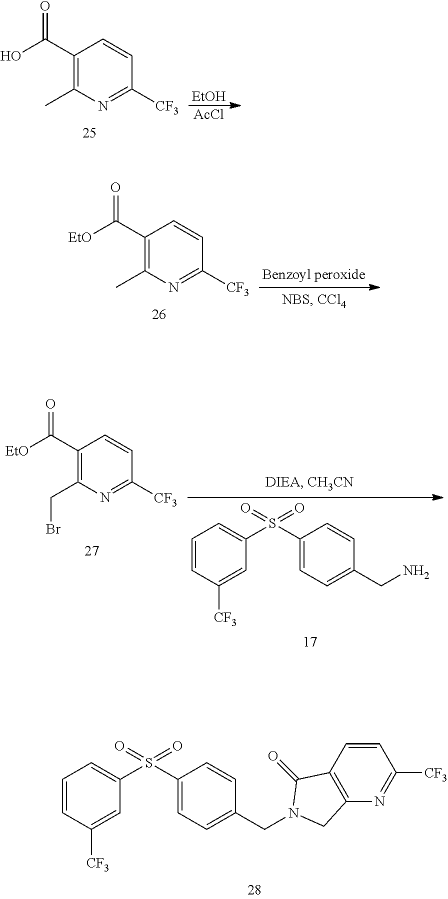

### Preparation of ethyl 2-methyl-6-(trifluoromethyl)nicotinate (26)

2-Methyl-6-(trifluoromethyl)nicotinic acid (25) (3.58 g, 17.5 mmol) was stirred in EtOH (50 mL) at rt. Acetyl chloride (AcCl; 2.48 mL, 34.9 mmol) was added dropwise, and the reaction was then heated to reflux for 6 hours. The reaction was concentrated in vacuo, the residue taken up in EtOAc, washed with saturated NaHCO₃ solution (twice), dried (Na₂SO₄), and the solvent removed in vacuo to give ethyl 2-methyl-6-(trifluoromethyl)nicotinate (26) (3.33 g, 82%); ¹H NMR (300 MHz, CDCl₃) δ 1.42 (t, 3H, J=7.26 Hz), 2.89 (s, 3H), 4.24 (q, 2H, J=7.26 Hz), 7.59 (d, 1H, J=8.58 Hz), 8.34 (d, 1H, J=8.14 Hz). The product was used without further purification.

### Preparation of ethyl 2-(bromomethyl)-6-(trifluoromethyl)nicotinate (27)

Ethyl 2-methyl-6-(trifluoromethyl)nicotinate (26) (3.33 g, 14.3 mmol), NBS (2.54 g, 14.3 mmol), and benzoyl peroxide (0.59 g, 4.3 mmol) were stirred under argon in dry CCl₄ (80 mL) at reflux for 16 hours. The reaction was washed with saturated NaHCO₃ solution, dried (Na₂SO₄), and the solvent was removed in vacuo to provide a 3:1 mixture of ethyl 2-(bromomethyl)-6-(trifluoromethyl)nicotinate (27) with starting material (4.07 g); ¹H NMR (300 MHz, CDCl₃) δ 1.26 (t, 3H, J=7.48 Hz), 4.29 (q, 2H, J=7.26 Hz), 4.85 (s, 2H), 7.51 (d, 1H, J=8.58 Hz), 8.25 (d, 1H, J=8.58 Hz). The crude product was used without additional purification or isolation.

### Preparation of 2-(trifluoromethyl)-6-(4-((3-(trifluoromethyl)phenyl)sulfonyl)benzyl)-6,7-dihydro-5H-pyrrolo[3,4-b]pyridin-5-one (28)

Crude ethyl 2-(bromomethyl)-6-(trifluoromethyl)nicotinate (27) (0.85 g, 2.72 mmol) and DIPEA (470 μL, 2.72 mmol) in MeCN (100 mL) was stirred with (4-((3-(trifluoromethyl)phenyl)sulfonyl)phenyl)methanamine (17) (0.57 g, 1.82 mmol) in DMF at room temperature for 72 hours and then at reflux for an additional 2 hours. The reaction was concentrated in vacuo. The residue was then taken up in EtOAc and washed sequentially with 1 M HCl, NaHCO₃ (saturated solution) and brine, dried (Na₂SO₄), and concentrated in vacuo. The crude product was purified by automated column chromatography (50% EtOAc/DCM), and the combined product fractions were combined and concentrated in vacuo. The residue was then taken up in DMSO (6 mL), filtered, and the residual solid was triturated in hot MeOH to give 2-(trifluoromethyl)-6-(4-((3-(trifluoromethyl)phenyl)sulfonyl)benzyl)-6,7-dihydro-5H-pyrrolo[3,4-b]pyridin-5-one (28); ¹H NMR (300 MHz, CDCl₃) δ 4.44 (s, 2H), 4.91 (s, 2H), 7.50 (d, 2H, J=8.19 Hz), 7.67 (t, 1H, J=7.77 Hz), 7.83 (d, 2H, J=7.86 Hz), 7.96 (d, 2H, J=8.25 Hz), 8.12 (d, 1H, J=7.65 Hz), 8.21 (s, 1H), 8.33 (d, J=7.89 Hz).

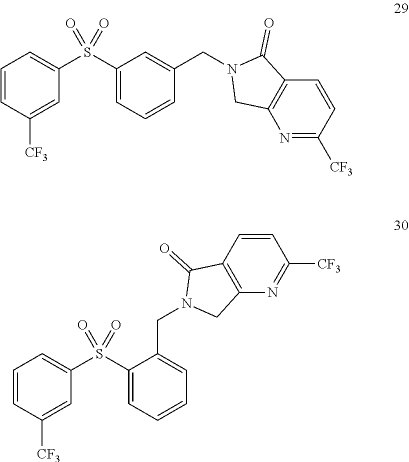 [SMILES: O=C1c2ccc(C(F)(F)F)nc2CN1Cc1cccc(S(=O)(=O)c2cccc(C(F)(F)F)c2)c1 ; O=C1c2ccc(C(F)(F)F)nc2CN1Cc1ccccc1S(=O)(=O)c1cccc(C(F)(F)F)c1]

2-(Trifluoromethyl)-6-(2-((3-(trifluoromethyl)phenyl)sulfonyl)benzyl)-6,7-dihydro-5H-pyrrolo[3,4-b]pyridin-5-one (29) and 2-(trifluoromethyl)-6-(3-((3-(trifluoromethyl)phenyl)sulfonyl)benzyl)-6,7-dihydro-5H-pyrrolo[3,4-b]pyridin-5-one (30) were prepared in an analogous fashion using (2-((3-(trifluoromethyl)phenyl) sulfonyl)phenyl)methanamine (18) or (3-((3-(trifluoromethyl)phenyl)sulfonyl) phenyl)methanamine respectively (19).

### Example 9

### Procedure for the synthesis of 2-(trifluoromethyl)-5-(3-((3-(trifluoromethyl)phenyl)sulfonyl)benzyl)-6,7-dihydropyrazolo[1,5-a]pyrazin-4(5H)-one (34)

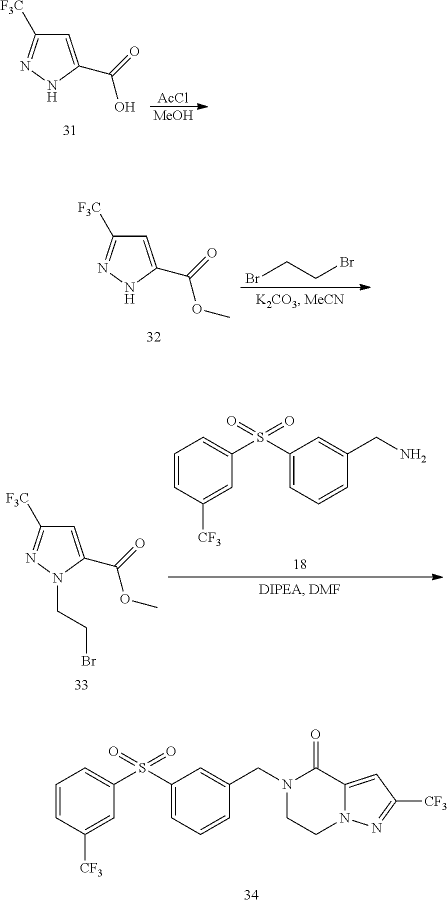

### Preparation of methyl 3-(trifluoromethyl)-1H-pyrazole-5-carboxylate (32)

3-(Trifluoromethyl)-1H-pyrazole-5-carboxylic acid (31) (1.0 g, 8.33 mmol) was stirred in MeOH (50 mL) at rt. AcCl (1.18 mL, 16.67 mmol) was added dropwise, and the reaction stirred at reflux for 2 hours. The reaction was concentrated in vacuo and partitioned between EtOAc and saturated NaHCO₃ solution. The organics were dried (Na₂SO₄) and concentrated in vacuo to give methyl 3-(trifluoromethyl)-1H-pyrazole-5-carboxylate (32) (1.0 g, 93%); ¹H NMR (300 MHz, CDCl₃) δ 3.98 (s, 3H), 7.10 (s, 1H). The product was used without purification.

### Preparation of methyl 1-(2-bromoethyl)-3-(trifluoromethyl)-1H-pyrazole-5-carboxylate (33)

Methyl-3-(trifluoromethyl)-1H-pyrazole-5-carboxylate (32) (1.0 g, 5.15 mmol), 1,2-dibromoethane (2.22 mL, 25.77 mmol), and K₂CO₃ (1.42 g, 10.31 mmol) were stirred in MeCN (50 mL) at reflux for 3 hours. The reaction was concentrated in vacuo. The residue was then partitioned between EtOAc and H₂O, and the organics were dried (Na₂SO₄) and concentrated in vacuo to give methyl 1-(2-bromoethyl)-3-(trifluoromethyl)-1H-pyrazole-5-carboxylate (33) (1.21 g, 78%); ¹H NMR (300 MHz, CDCl₃) δ 3.74 (t, 2H, J=6.78 Hz), 3.94 (s, 3H), 5.02 (t, 2H, J=6.75 Hz), 7.10 (s, 1H). The product was used without further purification

### Preparation of 2-(trifluoromethyl)-5-(3-((3-(trifluoromethyl)phenyl)sulfonyl)benzyl)-6,7-dihydropyrazolo[1,5-a]pyrazin-4(5H)-one (34)

Methyl 1-(2-bromoethyl)-3-(trifluoromethyl)-1H-pyrazole-5-carboxylate (33) (100 mg, 0.33 mmol), DIPEA (0.29 mL, 1.67 mmol) and (3-((3-(trifluoromethyl) phenyl)sulfonyl)phenyl)methanamine (18) (97 mg, 0.33 mmol) were stirred in DMF (3 mL) in a sealed vessel at 200° C. for 45 minutes in a microwave reactor. The reaction was concentrated in vacuo, and the residue purified by mass directed reverse phase HPLC to give 2-(trifluoromethyl)-5-(3-((3-(trifluoromethyl)phenyl)sulfonyl)benzyl)-6,7-dihydropyrazolo[1,5-a]pyrazin-4(5H)-one (34)

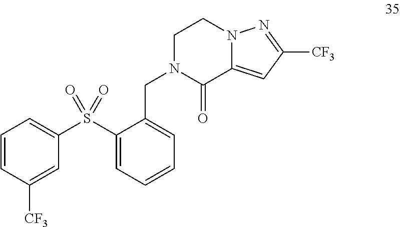 [SMILES: O=C1c2cc(C(F)(F)F)nn2CCN1Cc1ccccc1S(=O)(=O)c1cccc(C(F)(F)F)c1]

2-(trifluoromethyl)-5-(2-((3-(trifluoromethyl)phenyl)sulfonyl)benzyl)-6,7-dihydropyrazolo[1,5-a]pyrazin-4(5H)-one (35) was prepared in an analogous manner using (2-((3-(trifluoromethyl)phenyl)sulfonyl)phenyl)methanamine (19)

### Example 10

### General Coupling Protocols for Diarylsulfone Compounds

Stoichiometries given are to be considered exemplary and can be varied. Suitable organic bases may be used as alternates to TEA (e.g., DIPEA). Suitable coupling agents may be used as an alternative to HATU (e.g. EDC/HOBt). For HCl salts, at least one additional equivalent of base to that described must be employed. DCM may be substituted for DMF as solvent.

### a. General Coupling Protocol for the Synthesis of Compounds with General Structure (36)

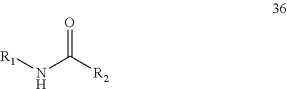

### Exemplified by the synthesis 2-(methylsulfonyl)-4-(trifluoromethyl)-N-(4-((3-(trifluoromethyl)phenyl)sulfonyl)benzyl)benzamide (38)

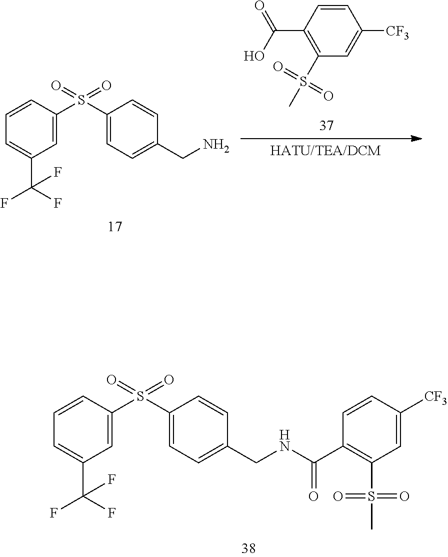 [SMILES: NCc1ccc(S(=O)(=O)c2cccc(C(F)(F)F)c2)cc1 ; CS(=O)(=O)c1cc(C(F)(F)F)ccc1C(=O)O ; CS(=O)(=O)c1cc(C(F)(F)F)ccc1C(=O)NCc1ccc(S(=O)(=O)c2cccc(C(F)(F)F)c2)cc1]

(4-((3-(Trifluoromethyl)phenyl)sulfonyl)phenyl)methanamine (17) (50 mg, 0.14 mmol), HATU (81 mg, 0.21 mmol), DIPEA (124 μL, 0.7 mmol), and 2-(methylsulfonyl)-4-(trifluoromethyl)benzoic acid (37) (49 mg, 0.18 mmol) were stirred in DMF (2 mL) at room temperature for 16 hours. The reaction was concentrated in vacuo, and the residue was purified by mass directed reverse phase HPLC to give 2-(methylsulfonyl)-4-(trifluoromethyl)-N-(4-((3-(trifluoromethyl)phenyl)sulfonyl)benzyl)benzamide (38).

### B. General protocol for BOC amino acids amide coupling exemplified by the synthesis (R)—N-(4-((3-(trifluoromethyl)phenyl)sulfonyl)benzyl)pyrrolidine-2-carboxamide (40)

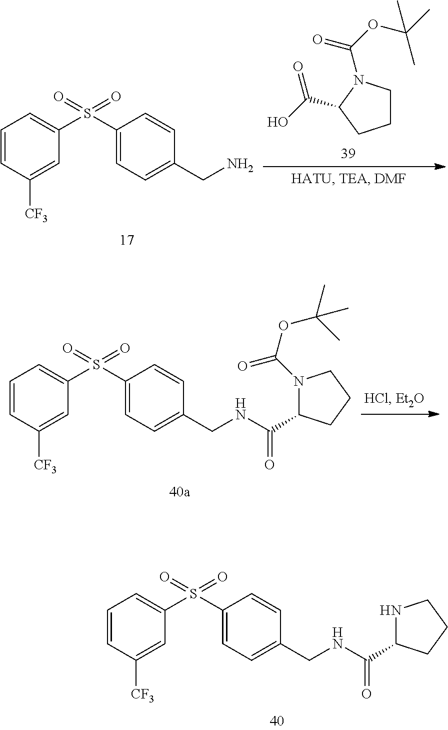 [SMILES: NCc1ccc(S(=O)(=O)c2cccc(C(F)(F)F)c2)cc1 ; CC(C)(C)OC(=O)N1CCC[C@@H]1C(=O)O ; CC(C)(C)OC(=O)N1CCC[C@@H]1C(=O)NCc1ccc(S(=O)(=O)c2cccc(C(F)(F)F)c2)cc1 ; O=C(NCc1ccc(S(=O)(=O)c2cccc(C(F)(F)F)c2)cc1)[C@H]1CCCN1]

(4-((3-(trifluoromethyl)phenyl)sulfonyl)phenyl)methanamine (17) (100 mg, 0.34 mmol), HATU (178 mg, 0.48 mmol), TEA (197 μL, 1.41 mmol), and (R)-1-(tert-butoxycarbonyl)pyrrolidine-2-carboxylic acid (40a) (87 mg, 0.34 mmol) were stirred in DMF (1 mL) at room temperature for 16 hours. The reaction was concentrated in vacuo, and the residue was treated with 2M HCl in Et₂O at room temperature for 5 hours. The reaction was then quenched with NaHCO₃ saturated solution, and the organics were separated, dried, and concentrated in vacuo. The residue was purified by mass directed reverse phase HPLC to give (R)—N-(4-((3-(trifluoromethyl)phenyl)sulfonyl)benzyl) pyrrolidine-2-carboxamide (40).

### C. B. General protocol for BOC amino acids amide coupling exemplified by the synthesis 2-(1-aminocyclohexyl)-N-(4-((3-(trifluoromethyl)phenyl)sulfonyl)benzyl) acetamide (41)

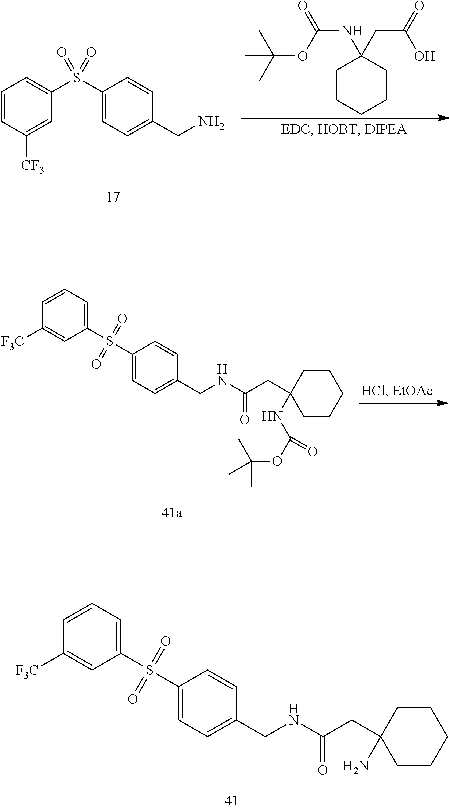 [SMILES: NCc1ccc(S(=O)(=O)c2cccc(C(F)(F)F)c2)cc1 ; CC(C)(C)OC(=O)NC1(CC(=O)O)CCCCC1 ; CC(C)(C)OC(=O)NC1(CC(=O)NCc2ccc(S(=O)(=O)c3cccc(C(F)(F)F)c3)cc2)CCCCC1 ; NC1(CC(=O)NCc2ccc(S(=O)(=O)c3cccc(C(F)(F)F)c3)cc2)CCCCC1]

(4-((3-(trifluoromethyl)phenyl)sulfonyl)phenyl)methanamine (17) (720 mg, 2.28 mmol), EDC (570 mg, 2.99 mmol), HOBT (410 mg, 2.99 mmol), DIPEA (640 μL, 3.89 mmol), and 2-(1-((tert-butoxycarbonyl)amino)cyclohexyl)acetic acid (588 mg, 2.28 mmol) were stirred in DMF (10 mL) at room temperature for 16 hours. The reaction was concentrated in vacuo. The residue was diluted with ethyl acetate (100 ml) and then washed sequentially with saturated NH₄Cl and saturated NaHCO₃. The organics were dried (Na₂SO₄) and then concentrated in vacuo, and the residue was purified by column chromatography using EtOAc:Hexane (1:1) to give the pure intermediate (41a). The material was further dissolved in ethyl acetate, and HCl gas was bubbled for two minutes to give the final product 2-(1-aminocyclohexyl)-N-(4-((3-(trifluoromethyl)phenyl)sulfonyl)benzyl)acetamide (41) with >98% purity.

### Example 11

### Procedure for the synthesis of 3-(methylsulfonyl)-5-(trifluoromethyl) picolinic acid (43)

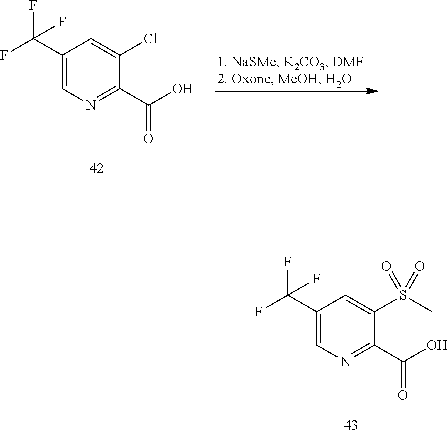 [SMILES: O=C(O)c1ncc(C(F)(F)F)cc1Cl ; CS(=O)(=O)c1cc(C(F)(F)F)cnc1C(=O)O]

3-Chloro-5-(trifluoromethyl)picolinic acid (42) (2.11 g, 10.0 mmol), K₂CO₃ (1.38 g, 10.0 mmol) and NaSMe (1.20 g, 25.0 mmol) were stirred in DMF (15 mL) at 110° C. for 16 hours. The reaction was concentrated in vacuo, and the residue was dissolved in MeOH (80 mL) and H₂O (80 mL). Oxone monopersulfate (30 g, 49 mmol) was added, and the reaction stirred at room temperature for 16 hours. The solid was removed by filtration, and the filtrate basified with 10% NaOH for 30 minutes. The MeOH was removed in vacuo. The aqueous portion acidified to pH 1 with 6 N HCl, extracted with EtOAc (3×80 mL), dried (Na₂SO₄), concentrated in vacuo, and the residue recrystallized (with 1 eq. DMF) from EtOAc/hexanes to give 3-(methylsulfonyl)-5-(trifluoromethyl) picolinic acid (43) as the DMF adduct (1.70 g, 51%); ¹H NMR (300 MHz, CD₃OD) δ 2.88 (s, 3H, DMF), 3.01 (s, 3H, DMF), 3.45 (s, 3H), 8.00 (s, 1H, DMF), 8.73 (s, 1H), 9.22 (s, 1H).

### Example 12

### Procedure for the synthesis of 2-(methylsulfonyl)-6-(trifluoromethyl) nicotinic acid (44)

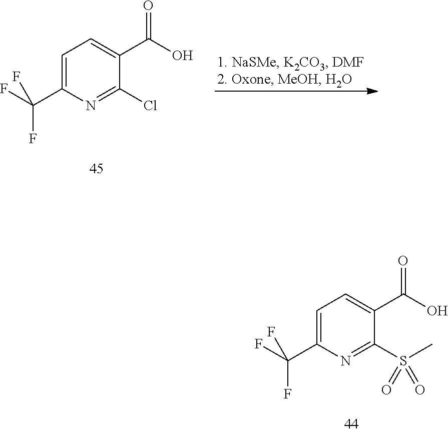 [SMILES: O=C(O)c1ccc(C(F)(F)F)nc1Cl ; CS(=O)(=O)c1nc(C(F)(F)F)ccc1C(=O)O]

3-(Methylsulfonyl)-5-(trifluoromethyl)picolinic acid (44) was prepared in an analogous fashion using 2-chloro-6-(trifluoromethyl)nicotinic acid (45) (5.35 g, 25.3 mmol) to give the required product (5.97 g, 69%; containing 1 equivalent of DMF); ¹H NMR (300 MHz, CD₃OD) δ 2.88 (s, 3H, DMF), 3.01 (s, 3H, DMF), 3.40 (s, 3H), 8.00 (s, 1H, DMF), 8.22 (d, 1H, J=7.5 Hz), 8.49 (d, 1H, J=7.5 Hz).

### Example 13

### Procedure for the synthesis of 2-(isopropylsulfonyl)-6-(trifluoromethyl) nicotinic acid (46)

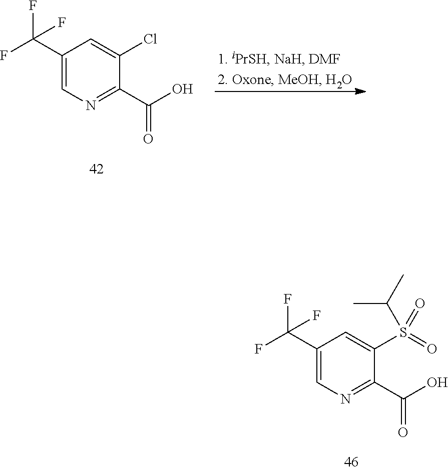 [SMILES: O=C(O)c1ncc(C(F)(F)F)cc1Cl ; CC(C)S(=O)(=O)c1cc(C(F)(F)F)cnc1C(=O)O]

2-(isopropylsulfonyl)-6-(trifluoromethyl)nicotinic acid (46) was prepared in an analogous fashion using 3-chloro-5-(trifluoromethyl)picolinic acid (42) (1.50 g, 7.09 mmol) to give the required (1.4 g, 62%); ¹H NMR (300 MHz, CDCl₃) δ 9.06 (s, 1H), 8.56 (s, 1H), 4.09 (m, 1H), 1.31 (d, 6H, J=6.8 Hz).

### Example 14

### Procedure for the synthesis of 2-(methylsulfonyl)-6-(trifluoromethyl) isonicotinic acid (49)

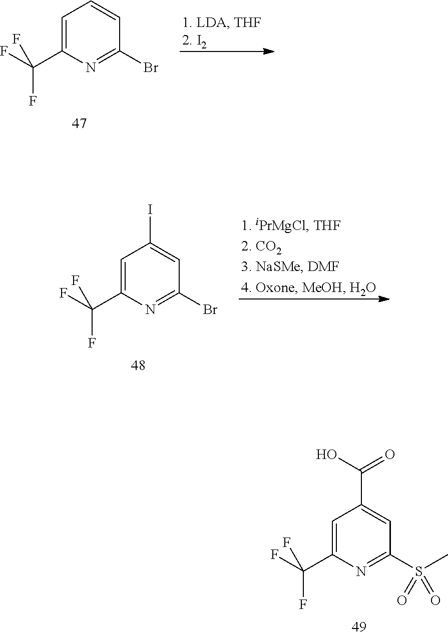 [SMILES: FC(F)(F)c1cccc(Br)n1 ; FC(F)(F)c1cc(I)cc(Br)n1 ; CS(=O)(=O)c1cc(C(=O)O)cc(C(F)(F)F)n1]

### Preparation of 2-bromo-4-iodo-6-(trifluoromethyl)pyridine (48)

Diisopropylamine (2.83 g, 28.0 mmol) was stirred under argon in dry THF (60 mL) at −85° C. nBuLi (1.6 M in hexanes, 17.5 mL, 28 mmol) was added dropwise, and the reaction stirred for 1 hour. 2-Bromo-6-(trifluoromethyl)pyridine (47) (3.00 g, 13.3 mmol) in dry THF (6 mL) was added dropwise, and the reaction stirred for 2 hours. I₂ (3.37 g, 13.3 mmol) was added in portions; the reaction was stirred for 30 minutes, quenched with H₂O, and extracted with EtOAc (3×30 mL). The organics were dried (Na₂SO₄), concentrated in vacuo, and purified by automated column chromatography (EtOAc/PE, 1:8) to give 2-bromo-4-iodo-6-(trifluoromethyl)pyridine (48) (2.3 g, 49%); ¹H NMR (300 MHz, CDCl₃) δ 7.98 (s, 1H), 8.03 (s, 1H).

### Preparation of 2-(methylsulfonyl)-6-(trifluoromethyl)isonicotinic acid (49)

2-Bromo-4-iodo-6-(trifluoromethyl)pyridine (48) (2.70 g, 7.67 mmol) was stirred under argon in dry THF (30 mL) at −10° C. ⁱPrMgCl (2.0 M, THF, 4.5 mL, 9.0 mmol) was added, and the mixture was stirred at 0° C. for 30 minutes. CO₂ was bubbled through the reaction, and stirring continued for 1.5 hours while allowing to warm to room temperature. The reaction was concentrated in vacuo, taken up in DMF (20 mL), and stirred with NaSMe (0.90 g, 19 mmol) at 100° C. for 2 hours. The reaction was concentrated in vacuo, the residue was taken up in MeOH (50 mL) and H₂O (50 mL) with oxone monopersulfate (30 g, 49 mmol), and the reaction stirred at room temperature for 3 hours. The reaction was filtered, the filtrate basified with 10% NaOH for 30 minutes, and the MeOH removed in vacuo. The aqueous residue was acidified with 6 N HCl and extracted with EtOAc (3×50 mL). The organics were dried (Na₂SO₄), concentrated in vacuo, and the residue recrystallized from EtOAc/hexanes with the presence of 1 eq. DMF to give 2-(methylsulfonyl)-6-(trifluoromethyl)isonicotinic acid (49) (1.70 g, 51%); ¹H NMR (300 MHz, CD₃OD) δ 2.88 (s, 3H, DMF), 3.01 (s, 3H, DMF), 3.34 (s, 3H), 8.00 (s, 1H, DMF), 8.52 (s, 1H), 8.73 (s, 1H).

### Example 15

### General procedure for the preparation of 6-phenoxypyridin-3-amines (12)

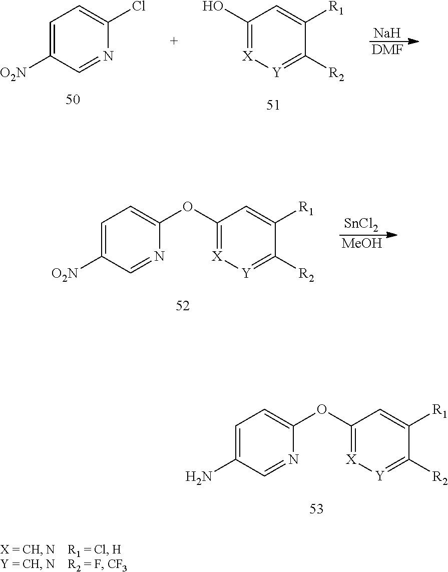

### Exemplified by the procedure for 6-(3-chloro-4-fluorophenoxy)pyridin-3-amine (15)

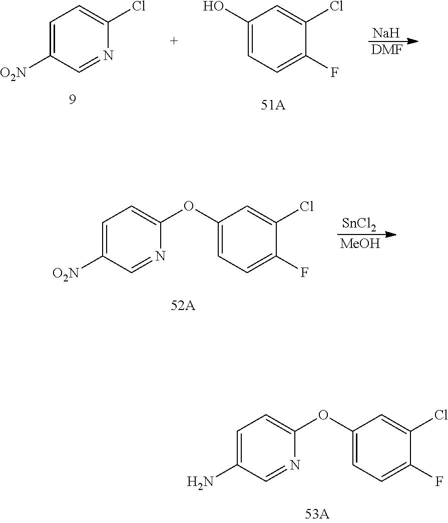

### Preparation of 2-(3-chloro-4-fluorophenoxy)-5-nitropyridine (52A)

2-Chloro-5-nitropyridine (50) (1.0 g, 6.31 mmol), 3-chloro-4-fluorophenol (51A) (0.92 g, 6.31 mmol), and NaH (60% dispersion in mineral oil; 250 mg, 6.9 mmol) were stirred under argon in DMF (20 mL) at reflux for 3 hours. The reaction was quenched with H₂O and extracted with EtOAc (3×10 mL). The organics were dried (Na₂SO₄), concentrated in vacuo, and the residue purified by automated flash chromatography (5% EtOAc/PE) to give 2-(3-chloro-4-fluorophenoxy)-5-nitropyridine (52A) (0.92 g. 54%). ¹H NMR (300 MHz, CDCl₃) δ 7.04-7.10 (m, 2H), 7.19-7.25 (m, 2H), 8.52 (dd, 1H, J=2.79, 9.00 Hz), 9.03 (d, 1H, J=2.55 Hz).

### Preparation of 6-(3-chloro-4-fluorophenoxy)pyridin-3-amine (53A)

2-(3-Chloro-4-fluorophenoxy)-5-nitropyridine (52A) (0.92 g, 3.4 mmol) and SnCl₂ (3.1 g, 13.73 mmol) were stirred in MeOH (15 mL) at reflux for 16 hours. The reaction was concentrated in vacuo, and the residue stirred in NaHCO₃(sat)/CH₂Cl₂ (1:1) at room temperature for 45 minutes. The resulting suspension was filtered through Celite, and the filtrate partitioned between CH₂Cl₂ and H₂O. The organics were dried (Na₂SO₄), concentrated in vacuo, and the residue purified by automated flash chromatography (5% EtOAc/Pet Ether) to give 6-(3-chloro-4-fluorophenoxy)pyridin-3-amine (53A) (0.43 g, 82%); ¹H NMR (300 MHz, CDCl₃) δ6.79 (d, 1H, J=8.58 Hz), 6.97 (m, 1H), 7.08 (m, 3H), 7.70 (d, 1H, J=2.88 Hz). LCMS m/z 238.8 (calcd. for C₁₁H₈ClFN₂O 238.0).

### Example 16

### General procedure for the synthesis of 2-methyl-2-(3-(substituted) phenylsulfonyl)propanoic acid exemplified by the synthesis of 2-methyl-2-((3-(trifluoromethyl)phenyl)sulfonyl)propanoic acid (58a)

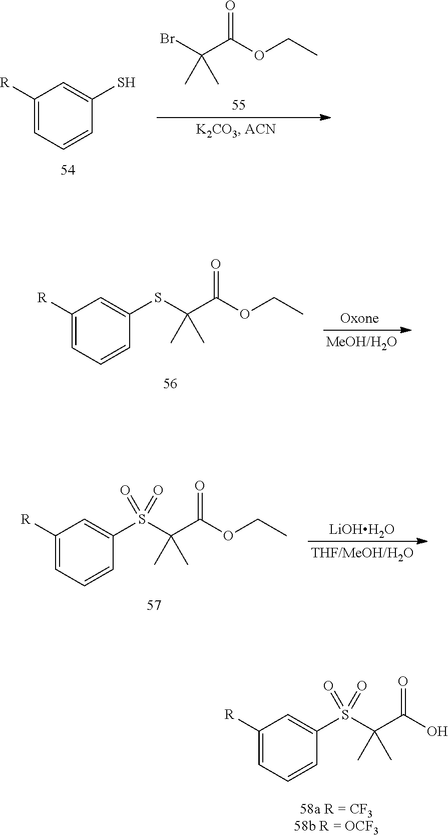

### Preparation of ethyl 2-methyl-2-(3-(trifluoromethyl)phenylthio)propanoate (56)

3-(Trifluoromethyl)benzenethiol (54) (25 g, 140.3 mmol), ethyl 2-bromo-2-methylpropanoate (55) (27.4 g, 140.3 mmol) and K₂CO₃ (24.2 g, 175.4 mmol) were heated at reflux in MeCN (400 mL) for 16 hours. The reaction was cooled, filtered, concentrated in vacuo and the residue purified by column chromatography (Pet Ether/DCM (80/20)) to give ethyl 2-methyl-2-(3-(trifluoromethyl)phenylthio)propanoate (56) (34.9 g, 85%); ¹H NMR (300 MHz-CD₃Cl) δ 1.49 (s, 6H), 3.65 (s, 3H), 7.45 (t, 1H, J=7.74 Hz), 7.63 (m, 2H), 7.07 (s, 1H).

### Preparation of ethyl 2-methyl-2-(3-(trifluoromethyl)phenylsulfonyl)propanoate (57)

Ethyl 2-methyl-2-(3-(trifluoromethyl)phenylthio)propanoate (56) (34.9 g, 119.4 mmol) and Oxone (220.2 g, 358.2 mmol) were stirred in H₂O/MeOH (330 mL/550 mL) at room temperature for 72 hours. The reaction was filtered, the MeOH removed in vacuo, and the aqueous layer extracted with EtOAc. The organics were dried (Na₂SO₄) and concentrated in vacuo to give ethyl 2-methyl-2-(3-(trifluoromethyl)phenylsulfonyl) propanoate (57) (38.4 g, 100%); ¹H NMR (300 MHz, CD₃Cl) δ 1.63 (s, 6H), 3.70 (s, 3H), 7.73 (t, 1H, J=7.86 Hz), 7.95 (d, 1H, J=7.83 Hz), 8.06 (d, 1H, J=7.98 Hz), 8.11 (s, 1H). The product was used without additional purification.

### Preparation of 2-methyl-2-(3-(trifluoromethyl)phenylsulfonyl)propanoic acid (58a)

Ethyl 2-methyl-2-(3-(trifluoromethyl)phenylsulfonyl)propanoate (57) (20 g, 61.7 mmol) and LiOH.H₂O (3.9 g, 92.5 mmol) were stirred in THF/MeOH/H₂O (175 mL, 3/1/1) at room temperature for 16 hours. The organics were removed in vacuo, and the aqueous portion acidified to pH 2 with 6M HCl and extracted with EtOAc. The organics were dried (Na₂SO₄) and concentrated in vacuo to give 2-methyl-2-(3-(trifluoromethyl) phenylsulfonyl)propanoic acid (58a) (17.1 g, 93%); ¹H NMR (300 MHz; CD₃Cl) δ 1.65 (s, 6H), 7.74 (t, 1H, J=7.71 Hz), 7.95 (d, 1H, J=7.83 Hz), 8.12 (d, 1H, J=8.04 Hz), 8.16 (s, 1H). The product was used without further purification.

### Example 17

### Procedure for the synthesis of 2-methyl-2-((3-(trifluoromethyl)phenyl) sulfonyl)propan-1-amine (60)

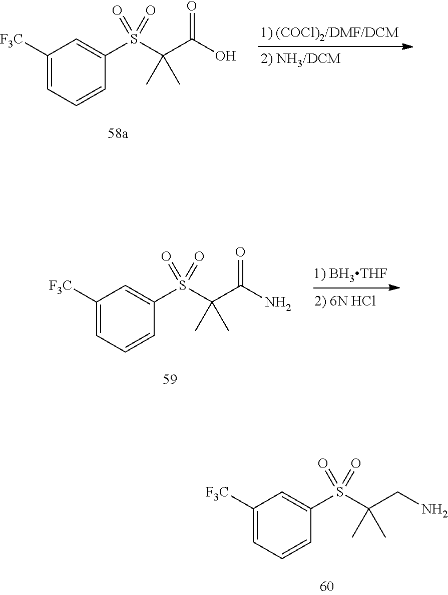 [SMILES: CC(C)(C(=O)O)S(=O)(=O)c1cccc(C(F)(F)F)c1 ; CC(C)(C(N)=O)S(=O)(=O)c1cccc(C(F)(F)F)c1 ; CC(C)(CN)S(=O)(=O)c1cccc(C(F)(F)F)c1]

### Preparation of 2-methyl-2-O-(trifluoromethyl)phenyl)sulfonyl)propanamide (59)

2-Methyl-2-(3-(trifluoromethyl)phenylsulfonyl)propanoic acid (58a) (4.86 g, 16.4 mmol) and oxalyl chloride (4.3 mL, 48.5 mmol) were stirred in dry CH₂Cl₂ (100 mL) at room temperature under Ar. DMF (cat) was added, and the reaction was stirred at room temperature for 1 hour. The solvent was removed in vacuo, dried under high vacuum for 2 hours, and the residue was then taken up in dry CH₂Cl₂ (50 mL). NH₃ (gas) was bubbled through the reaction for 10 minutes, and the reaction was then stirred at room temperature for 16 hours. The reaction was diluted with DCM (50 mL) and washed sequentially with 1 N HCl, NaHCO₃ (saturated solution), and brine. The organics were dried (Na₂SO₄) and concentrated in vacuo to give 2-methyl-2-((3-(trifluoromethyl)phenyl) sulfonyl)propanamide (59) (4.83 g, 100%); ¹H NMR (300 MHz-CD₃Cl) δ 1.54 (s, 6H), 5.75 (bs, 1H), 6.83 (bs, 1H), 7.67 (t, 1H, J=7.83 Hz), 7.89 (d, 1H, J=7.77 Hz). 8.02 (d, 1H, J=7.86 Hz), 8.08 (s, 1H). The product was used without further purification.

### Preparation of 2-methyl-2-((3-(trifluoromethyl)phenyl)sulfonyl)propan-1-amine (60)

2-Methyl-2-((3-(trifluoromethyl)phenyl)sulfonyl)propanamide (59) (4.83 g, 16.4 mmol) and BH₃.THF (1M solution; 52 ml, 52 mmol) were stirred in dry THF (75 mL) under Ar at reflux for 3 hours. The reaction was cooled, 6 N HCl (26 mL) added, and then the reaction was heated at reflux for 1 hour. The reaction was concentrated in vacuo, and the residue was taken up in H₂O (30 mL) and washed with Et₂O. The aqueous layer was filtered, and the filtrate basified with NaOH (7 g). The reaction was extracted with DCM, and the organics were dried (Na₂SO₄) and concentrated in vacuo to give 2-methyl-2-((3-(trifluoromethyl)phenyl)sulfonyl)propan-1-amine (60) (2.9 g, 63%); ¹H NMR (300 MHz-CD₃Cl) δ 1.31 (s, 6H), 2.98 (s, 2H), 7.74 (m, 1H), 7.95 (m, 1H), 8.12 (m, 2H). The product was used without further purification.

### Example 18

### Procedure for the synthesis of N,3-dimethyl-3-((3-(trifluoromethyl) phenyl)sulfonyl)butan-1-amine hydrochloride (71)

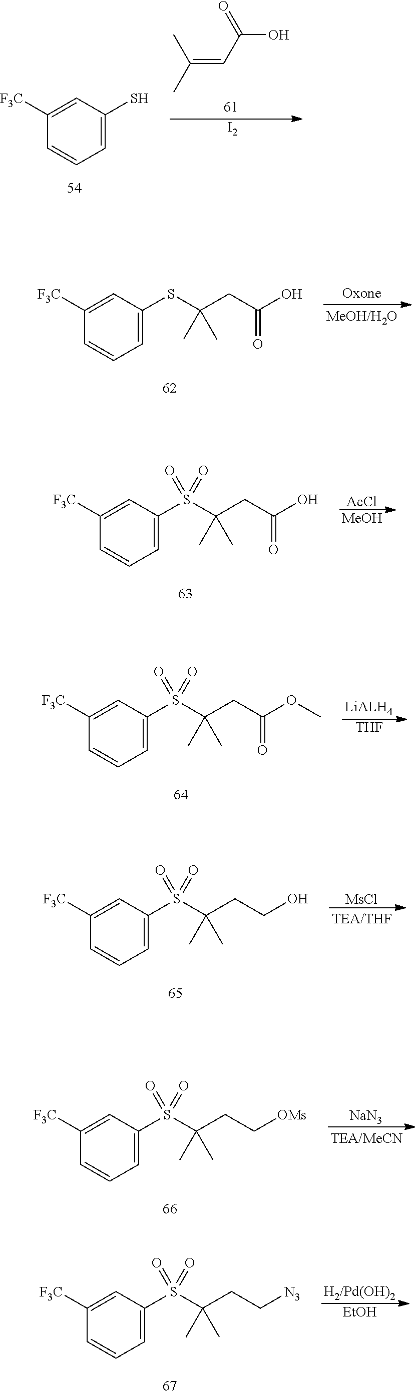

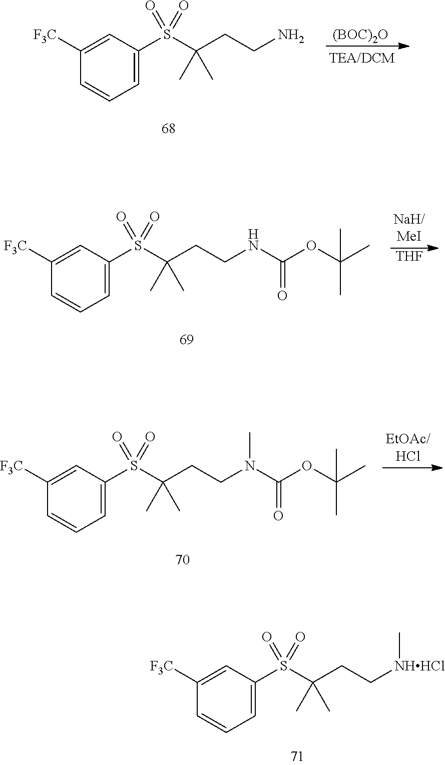

### Preparation of 3-methyl-3-((3-(trifluoromethyl)phenyl)thio)butanoic acid (62)

3 Trifluoromethylthiophenol (16) (25 g, 140 mmol), 3,3-dimethylacrylic acid (61) (14.0 g, 140 mmol) and iodine (6.9 g, 27 mmol) were heated under Ar at 105° C. for 3 hours. The reaction was cooled, taken up in EtOAc (300 mL) and washed with Na₂S₂SO₃ (saturated solution) (3×100 mL). The organics were separated, dried (MgSO₄), concentrated in vacuo and the residue purified by automated column chromatography (3% EtOAc/Pet ether) to give 3-methyl-3-((3-(trifluoromethyl)phenyl)thio)butanoic acid (62) (30.61 g, 78.6%); ¹H NMR (300 MHz-CD₃Cl) δ 1.43 (s, 6H), 2.55 (s, 2H), 7.49 (t, 1H, J=7.68 Hz), 7.65 (d, 1H, J=7.8 Hz), 7.78 (d, 1H, J=7.71 Hz), 7.84 (s, 1H).

### Preparation of 3-methyl-3-((3-(trifluoromethyl)phenyl)sulfonyl)butanoic acid (63)

3-Methyl-3-((3-(trifluoromethyl)phenyl)thio)butanoic acid (62) (14.0 g, 50 mmol) and oxone (83 g, 135 mmol) were stirred in MeOH/H₂O (150/100 mL) at room temperature for 16 hours. The reaction was filtered, the MeOH removed in vacuo, and the aqueous extracted with DCM (3×75 mL). The organics were dried (MgSO₄) and concentrated in vacuo to give 3-methyl-3-((3-(trifluoromethyl)phenyl)sulfonyl)butanoic acid (63) (8.62 g, 56%); ¹H NMR (300 MHz-CD₃Cl) δ 1.51 (s, 6H), 2.77 (s, 2H), 7.77 (t, 1H, J=7.77 Hz), 7.97 (d, 1H, J=7.74 Hz), 8.11 (d, 1H, J=7.92 Hz), 8.17 (s, 1H). The product was used without further purification.

### Preparation of methyl 3-methyl-3-((3-(trifluoromethyl)phenyl)sulfonyl) butanoate (64)

3-Methyl-3-((3-(trifluoromethyl)phenyl)sulfonyl)butanoic acid (63) (11.1 g, 35.5 mmol) was stirred in MeOH (75 mL) at 0° C. Acetyl chloride (3.6 mL, 53.2 mmol) was added dropwise, and the reaction heated at reflux for 2 hours. The MeOH was removed in vacuo, and the residue was taken up in EtOAc (150 mL) and washed with NaHCO₃ (saturated solution; 2×100 mL). The organics were separated, dried (MgSO₄), and concentrated in vacuo to give methyl 3-methyl-3-((3-(trifluoromethyl)phenyl)sulfonyl) butanoate (64) (10.2 g, 89%); ¹H NMR (300 MHz-CD₃Cl) δ 1.48 (s, 6H), 2.72 (s, 2H), 3.69 (s, 3H), 7.76 (t, 1H, J=7.8 Hz), 7.96 (d, 1H, J=7.77 Hz), 8.10 (d, 1H, J=7.86 Hz), 8.16 (s, 1H). The product was used without additional purification.

### Preparation of 3-methyl-3-O-(trifluoromethyl)phenyl)sulfonyl)butan-1-ol (65)

Methyl 3-methyl-3-((3-(trifluoromethyl)phenyl)sulfonyl) butanoate (64) (10.2 g, 26.3 mmol) was taken up in dry THF under Ar at 0° C. LiALH₄ (1.33 g, 35 mmol) was added in portions, and the reaction stirred for 30 minutes at room temperature. The reaction was quenched with 1 M NaOH, the precipitate was removed by filtration, and the filtrate was concentrated in vacuo. The residue was taken up in EtOAc and washed sequentially with NH₄Cl (saturated solution), NaHCO₃ (saturated solution), and brine. The layers were separated, and the organics were dried (MgSO₄) and concentrated in vacuo to give 3-methyl-3-((3-(trifluoromethyl)phenyl) sulfonyl)butan-1-ol (65) (7.78 g, 84.7%); ¹H NMR (300 MHz-CD₃Cl) δ 1.35 (s, 6H), 2.00 (t, 2H J=6.57 Hz), 3.84 (t, 2H, J=6.48 Hz), 7.73 (t, 1H, J=7.83 Hz), 7.93 (d, 1H, J=7.83 Hz), 8.09 (d, 1H, J=7.92 Hz), 8.14 (s, 1H). The product was used without additional purification.

### Preparation of 3-methyl-3-((3-(trifluoromethyl)phenyl)sulfonyl)butyl methanesulfonate (66)

3-Methyl-3-((3-(trifluoromethyl)phenyl)sulfonyl)butan-1-ol (65) (7.78 g, 26.3 mmol) and TEA (7.4 mL, 52.6 mmol) were stirred in dry THF at 0° C. under Ar. MsCl (2.5 mL, 31.6 mmol) was added dropwise, and the reaction stirred for 30 minutes while allowing to warm to room temperature. The precipitate was removed by filtration, the filtrate concentrated in vacuo, and the residue taken up in DCM (150 mL). The organics were washed sequentially with NH₄Cl (saturated solution), NaHCO₃ (saturated solution) and brine. The layers were separated, and the organic layer was dried (MgSO₄) and concentrated in vacuo to give 3-methyl-3-((3-(trifluoromethyl)phenyl)sulfonyl)butyl methanesulfonate (66) (9.8 g, 100%); ¹H NMR (300 MHz-CD₃Cl) δ 1.31 (s, 6H), 2.16 (t, 2H, J=6.96 Hz), 2.96 (s, 2H), 4.42 (t, 2H, J=6.96 Hz), 7.69 (t, 1H, J=7.83 Hz), 7.86 (d, 1H, J=7.80 Hz), 8.02 (d, 1H, J=7.95 Hz), 8.07 (s, 1H). The product was used without additional purification.

### Preparation of 1-((4-azido-2-methylbutan-2-yl)sulfonyl)-3-(trifluoromethyl)benzene (67)

3-Methyl-3-((3-(trifluoromethyl)phenyl)sulfonyl)butyl methanesulfonate (66) (3.12 g, 8.3 mmol), NaN₃ (1.04 g, 16 mmol), and TEA (3.3 mL, 24 mmol) were heated at reflux in MeCN for 16 hours. The reaction was cooled, concentrated in vacuo, and the residue partitioned between EtOAc and H₂O. The organics were dried (MgSO₄) and concentrated in vacuo. The residue was purified by automated column chromatography (20% EtOAc/Pet Ether) to give 1-((4-azido-2-methylbutan-2-yl)sulfonyl)-3-(trifluoromethyl)benzene (67) (2.26 g, 85.0%); ¹H NMR (300 MHz-CD₃Cl) δ 1.27 (s, 6H), 1.92 (t, 2H, J=7.47 Hz), 3.43 (t, 2H, J=7.89 Hz), 7.69 (t, 1H, J=7.83 Hz), 7.88 (d, 1H, J=7.83 Hz), 8.01 (d, 1H, J=7.92 Hz), 8.06 (s, 1H).

### Preparation of 3-methyl-3-((3-(trifluoromethyl)phenyl)sulfonyl)butan-1-amine (68)

1-((4-Azido-2-methylbutan-2-yl)sulfonyl)-3-(trifluoromethyl)benzene (67) (1 g, 3.1 mmol) and Pd(OH)₂ (10% w/w) were taken up in EtOH and hydrogenated in a Parr apparatus under an H₂ atmosphere (50 PSI) for 1 hour. The catalyst was removed by multiple filtrations, and the filtrate was concentrated in vacuo to give 3-methyl-3-((3-(trifluoromethyl)phenyl)sulfonyl)butan-1-amine (68) (800 mg, 90%). The product was confirmed with positive ion mode LCMS and FIA MS and used without further purification.

### Preparation of tert-butyl (3-methyl-3-((3-(trifluoromethyl)phenyl sulfonyl)butyl) carbamate (69)

3-Methyl-3-((3-(trifluoromethyl)phenyl)sulfonyl)butan-1-amine (30) (750 mg, 2.5 mmol), di-tert-butyl dicarbonate (522 mg, 3.0 mmol), and TEA (697 μL, 5.0 mmol) were stirred in DCM (50 mL) at room temperature for 1 h. The reaction was concentrated in vacuo and the crude residue purified by automated column chromatography (20% EtOAc/Pet Ether) to give tert-butyl (3-methyl-3-((3-(trifluoromethyl)phenyl) sulfonyl)butyl)carbamate (69) (680 mg, 70%); ¹H NMR (300 MHz-CD₃Cl) δ 1.35 (s, 6H), 1.43 (s, 9H), 1.94 (m, 2H), 3.31 (m, 2H), 4.65 (bs, 1H), 7.74 (t, 1H, J=7.92 Hz), 7.94 (d, 1H, J=7.89 Hz), 8.09 (d, 1H, J=7.89 Hz), 8.14 (s, 1H).

### Preparation of tert-butyl methyl(3-methyl-3-((3-(trifluoromethyl)phenyl)sulfonyl) butyl)carbamate (70)

tert-Butyl (3-methyl-3-((3-(trifluoromethyl)phenyl) sulfonyl)butyl)carbamate (69) (680 mg, 1.9 mmol) was stirred in dry THF under Ar at room temperature. NaH (60% dispersion in oil; 90 mg, 2.3 mmol) was added, and the reaction stirred for 30 minutes. MeI (140 μL, 2.3 mmol) was added. The reaction stirred at room temperature for 16 hours, and then the reaction was quenched with H₂O and concentrated in vacuo. The residue was partitioned between DCM and H₂O. The organics were separated, washed sequentially with NH₄Cl (saturated solution), NaHCO₃ (saturated solution) and brine, dried (MgSO₄) and concentrated in vacuo to give tert-butyl methyl(3-methyl-3-((3-(trifluoromethyl)phenyl)sulfonyl)butyl)carbamate (70) (690 mg, 90.1%); ¹H NMR (300 MHz-CD₃Cl) δ 1.22 (s, 6H), 1.35 (s, 9H), 1.86 (m, 2H), 2.78 (s, 3H), 3.29 (bs, 2H), 7.67 (t, 1H, J=7.80 Hz), 7.83 (d, 1H, J=7.71 Hz), 8.02 (d, 1H, J=7.86 Hz), 8.07 (s, 1H).

### Preparation of N,3-dimethyl-3-((3-(trifluoromethyl)phenyl) sulfonyl)butan-1-amine hydrochloride (71)

tert-Butyl methyl(3-methyl-3-((3-(trifluoromethyl)phenyl)sulfonyl)butyl)carbamate (70) (690 mg, 1.68 mmol) was taken up in EtOAc (40 mL). HCl gas was passed through the solution at room temperature for 5 minutes, and stirring then continued for 15 minutes. The reaction was concentrated in vacuo to give N,3-dimethyl-3-((3-(trifluoromethyl)phenyl) sulfonyl)butan-1-amine hydrochloride (71) (472 mg, 82%). The product was confirmed with positive ion mode LCMS and FIA MS and used without further purification.

### Example 19

### Procedure for the synthesis of 3-((3-fluoro-5-(trifluoromethyl)phenyl) sulfonyl)-3-methylbutan-1-amine (72)

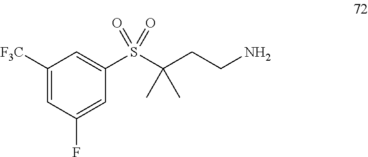 [SMILES: CC(C)(CCN)S(=O)(=O)c1cc(F)cc(C(F)(F)F)c1]

### Preparation of N 3-(3-fluoro-5-(trifluoromethyl)phenylthio)-3-methylbutanoic acid (75)

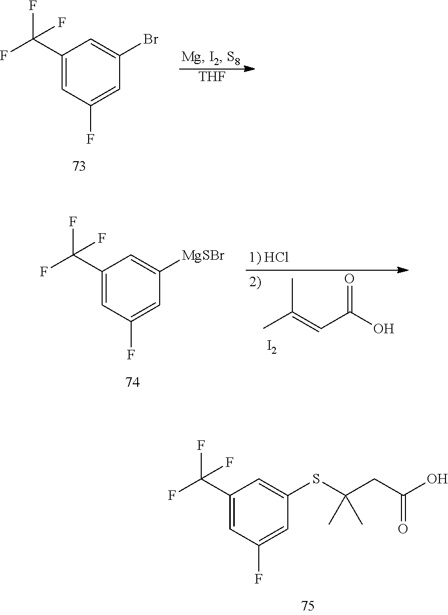

### Preparation of 3-fluoro-5-(trifluoromethyl)benzenethiol magnesium bromide (74)

Mg ribbon (1.09 g, 44.9 mmol) (cleaned with hexane/Et₂O) and I₂ (initiator) was stirred in dry THF (75 mL) at room temperature. 1-Bromo-3-fluoro-5-(trifluoromethyl)benzene (73) (10.0 g, 41.2 mmol) was added dropwise, and the reaction stirred for 2 hours at room temperature (reaction initiated with heat gun). Sulfur (1.32 g, 41.2 mmol) was added, and the reaction stirred at room temperature for 2 hours. The reaction was filtered, and the filtrate concentrated in vacuo to give crude 3-fluoro-5-(trifluoromethyl)benzenethiol magnesium bromide (74) which was used without purification.

### Preparation of 3-(3-fluoro-5-(trifluoromethyl)phenylthio)-3-methylbutanoic acid (75)

Crude 3-fluoro-5-(trifluoromethyl)benzenethiol magnesium bromide (74) (5.03 g, 26.7 mmol) was partitioned between 1 M HCl and Et₂O. The organics were separated, dried, and concentrated in vacuo. 3,3-Dimethylacyrlic acid (2.67 g, 26.7 mmol), and I₂ (2.25 g, 8.9 mmol) were added. The reaction was heated at 100° C. for 3 hours. After cooling, the reaction mixture was taken up in EtOAc and washed with saturated sodium metabisulphite solution until the reaction decolored. The organics were separated, dried, and concentrated in vacuo. The residue was purified by automated column chromatography (8% PE/EtOAc) to give 3-(3-fluoro-5-(trifluoromethyl)phenylthio)-3-methylbutanoic acid (75) (2.0 g, 25%); ¹H NMR (300 MHz-CD₃Cl) δ 1.46 (s, 6H), 2.58 (s, 2H), 7.37 (d, 1H, J=8.01 Hz), 7.53 (d, 1H, J=8.07 Hz), 7.65 (s, 1H).

### Preparation of 3-(3-fluoro-5-(trifluoromethyl)phenylsulfonyl)-3-methylbutan-1-amine (72)

3-(3-Fluoro-5-(trifluoromethyl)phenylsulfonyl)-3-methylbutan-1-amine (72) was prepared in analogous fashion to afford 3-methyl-3-(3-(trifluoromethyl)phenylsulfonyl) butan-1-amine (68) using 3-(3-fluoro-5-(trifluoromethyl)phenylthio)-3-methylbutanoic acid (75).

### Example 20

### Procedure for the synthesis of 6-(2-methyl-2-((3-(trifluoromethyl) phenyl)sulfonyl)propyl)-2-(trifluoromethyl)-6,7-dihydro-5H-pyrrolo[3,4-b]pyridin-5-one (79)

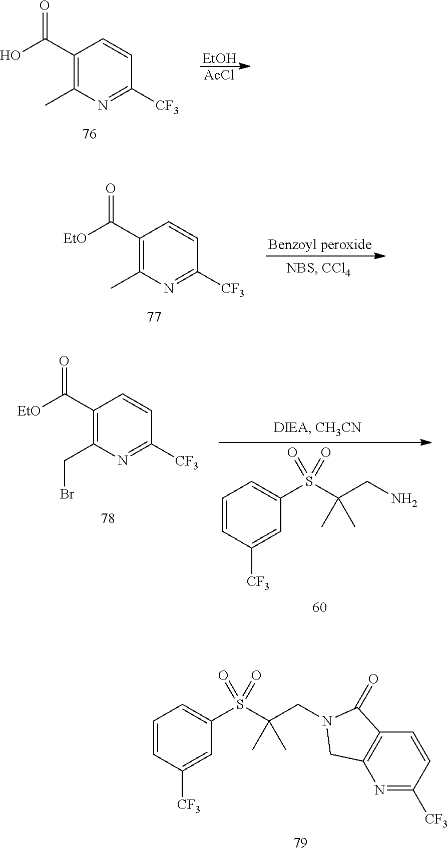

### Preparation of ethyl 2-methyl-6-(trifluoromethyl)nicotinate (77)

2-Methyl-6-(trifluoromethyl)nicotinic acid (76) (3.58 g, 17.5 mmol) was stirred in EtOH (50 mL) at room temperature. AcCl (2.48 mL, 34.9 mmol) was added dropwise, and the reaction was then heated to reflux for 6 hours. At this time, the reaction was concentrated in vacuo. The residue was then taken up in EtOAc, washed with saturated NaHCO₃ solution (twice), dried (Na₂SO₄), and the solvent removed in vacuo to give ethyl 2-methyl-6-(trifluoromethyl)nicotinate (77) (3.33 g, 82%); ¹H NMR (300 MHz, CDCl₃) δ 1.42 (t, 3H, J=7.26 Hz), 2.89 (s, 3H), 4.24 (q, 2H, J=7.26 Hz), 7.59 (d, 1H, J=8.58 Hz), 8.34 (d, 1H, J=8.14 Hz). The product was used without further purification.

### Preparation of ethyl 2-(bromomethyl)-6-(trifluoromethyl)nicotinate (78)

Ethyl 2-methyl-6-(trifluoromethyl)nicotinate (77) (3.33 g, 14.3 mmol), NBS (2.54 g, 14.3 mmol), and benzoyl peroxide (0.59 g, 4.3 mmol) were stirred under argon in dry CCl₄ (80 mL) at reflux for 16 hours. The reaction was washed with saturated NaHCO₃ solution, dried (Na₂SO₄), and the solvent was removed in vacuo to provide a 3:1 mixture of ethyl 2-(bromomethyl)-6-(trifluoromethyl)nicotinate (78) with starting material (4.07 g); ¹H NMR (300 MHz, CDCl₃) δ 1.26 (t, 3H, J=7.48 Hz), 4.29 (q, 2H, J=7.26 Hz), 4.85 (s, 2H), 7.51 (d, 1H, J=8.58 Hz), 8.25 (d, 1H, J=8.58 Hz). The crude product was used without purification or isolation.

### Preparation of 6-(2-methyl-2-((3-(trifluoromethyl)phenyl)sulfonyl)propyl)-2-(trifluoromethyl)-6,7-dihydro-5H-pyrrolo[3,4-b]pyridin-5-one (79)

Crude ethyl 2-(bromomethyl)-6-(trifluoromethyl)nicotinate (78) (120 mg, 0.38 mmol), DIEA (0.167 μL, 0.96 mmol), and 2-methyl-2-((3-(trifluoromethyl)phenyl) sulfonyl) propan-1-amine (60) (54 mg, 0.19 mmol) were heated in CH₃CN at 120° C. for 25 minutes, then 130° C. for 30 minutes in a microwave reactor. The reaction was concentrated and purified by mass directed reverse phase HPLC to give 6-(cis-4-fluoro-4-(3-(trifluoromethyl)phenylsulfonyl)cyclohexyl)-2-(trifluoro-methyl)-6,7-dihydro-5H-pyrrolo[3,4-b]pyridin-5-one (79)

### Example 21

### Procedure for the synthesis of 6-(3-methyl-3-((3-(trifluoromethyl)phenyl) sulfonyl)butyl)-2-(trifluoromethyl)-6,7-dihydro-5H-pyrrolo[3,4-b]pyridin-5-one (80)

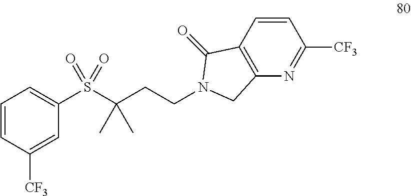 [SMILES: CC(C)(CCN1Cc2nc(C(F)(F)F)ccc2C1=O)S(=O)(=O)c1cccc(C(F)(F)F)c1]

6-(3-Methyl-3-((3-(trifluoromethyl)phenyl)sulfonyl)butyl)-2-(trifluoromethyl)-6,7-dihydro-5H-pyrrolo[3,4-b]pyridin-5-one (81) was synthesized in an analogous manner to 6-(2-methyl-2-((3-(trifluoromethyl)phenyl)sulfonyl)propyl)-2-(trifluoromethyl)-6,7-dihydro-5H-pyrrolo[3,4-b]pyridin-5-one (79) using 3-methyl-3-((3-(trifluoromethyl)phenyl)sulfonyl)butan-1-amine (68)

### Example 22

### Procedure for the synthesis of 5-(3-methyl-3-((3-(trifluoromethyl) phenyl)sulfonyl)butyl)-2-(trifluoromethyl)-6,7-dihydropyrazolo[1,5-a]pyrazin-4(5H)-one (85)

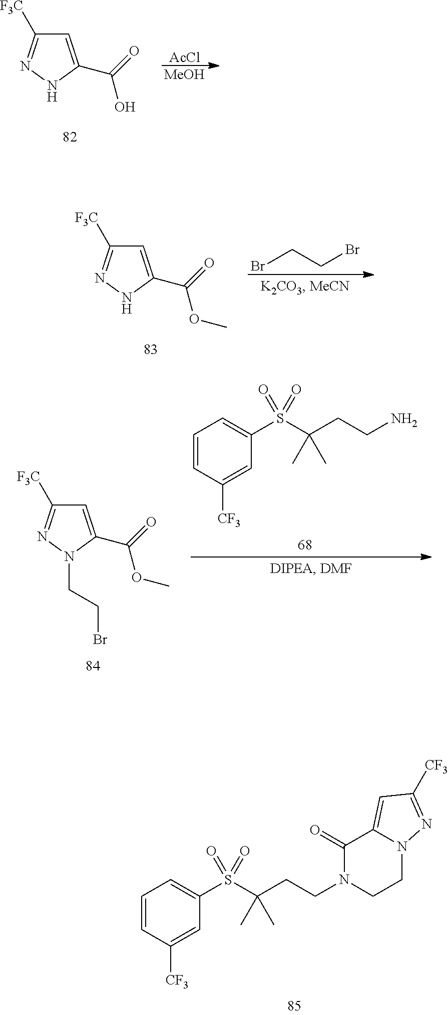

### Preparation of methyl 3-(trifluoromethyl)-1H-pyrazole-5-carboxylate (83)

3-(Trifluoromethyl)-1H-pyrazole-5-carboxylic acid (82) (1.0 g, 8.33 mmol) was stirred in MeOH (50 mL) at room temperature. AcCl (1.18 mL, 16.67 mmol) was added dropwise, and the reaction stirred at reflux for 2 hours. The reaction was concentrated in vacuo and partitioned between EtOAc and saturated NaHCO₃ solution. The organics were dried (Na₂SO₄) and concentrated in vacuo to give methyl 3-(trifluoromethyl)-1H-pyrazole-5-carboxylate (83) (1.0 g, 93%); NMR (300 MHz, CDCl₃) δ 3.98 (s, 3H), 7.10 (s, 1H). The product was used without purification.

### Preparation of methyl 1-(2-bromoethyl)-3-(trifluoromethyl)-1H-pyrazole-5-carboxylate (84)

Methyl-3-(trifluoromethyl)-1H-pyrazole-5-carboxylate (83) (1.0 g, 5.15 mmol), 1,2-dibromoethane (2.22 mL, 25.77 mmol) and K₂CO₃ (1.42 g, 10.31 mmol) were stirred in MeCN (50 mL) at reflux for 3 hours. The reaction was concentrated in vacuo, and the residue partitioned between EtOAc and H₂O. The organics were dried (Na₂SO₄) and concentrated in vacuo to give methyl 1-(2-bromoethyl)-3-(trifluoromethyl)-1H-pyrazole-5-carboxylate (84) (1.21 g, 78%); ¹H NMR (300 MHz, CDCl₃) δ 3.74 (t, 2H, J=6.78 Hz), 3.94 (s, 3H), 5.02 (t, 2H, J=6.75 Hz), 7.10 (s, 1H). The product was used without further purification

### Preparation of 5-(3-methyl-3-((3-(trifluoromethyl)phenyl)sulfonyl)butyl)-2-(trifluoromethyl)-6,7-dihydropyrazolo[1,5-a]pyrazin-4(5H)-oneamine (85)

Methyl 1-(2-bromoethyl)-3-(trifluoromethyl)-1H-pyrazole-5-carboxylate (84) (100 mg, 0.33 mmol), DIPEA (0.29 mL, 1.67 mmol) and 3-methyl-3-((3-(trifluoromethyl) phenyl)sulfonyl)butan-1-amine (68) (97 mg, 0.33 mmol) were stirred in DMF (3 mL) in a sealed vessel at 200° C. for 45 minutes in a microwave reactor. The reaction was concentrated in vacuo, and the residue purified by mass directed reverse phase HPLC to give 5-(3-methyl-3-((3-(trifluoromethyl)phenyl)sulfonyl)butyl)-2-(trifluoromethyl)-6,7-dihydropyrazolo[1,5-a]pyrazin-4(5H)-one (85).

### Example 23

### General Coupling Protocols

Stoichiometries given are to be considered exemplary and can be varied. Suitable organic bases may be used as alternates to TEA (e.g., DIPEA). Suitable coupling agents may be used as an alternative to HATU (e.g. EDC/HOBt). For HCl salts, at least one additional equivalent of base to that described must be employed. DMF may be substituted for CH₂Cl₂ as solvent.

### (A) General Coupling Protocol for the Synthesis of Compounds with General Structure (86)

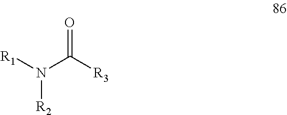

### Exemplified by the synthesis N-(6-(4-fluoro-3-(trifluoromethyl)phenoxy)pyridin-3-yl)-2-methyl-2-((3-(trifluoromethoxy)phenyl)sulfonyl)propanamide (88)

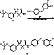 [SMILES: CC(C)(C(=O)O)S(=O)(=O)c1cccc(OC(F)(F)F)c1 ; Nc1ccc(Oc2ccc(F)c(Cl)c2)nc1 ; CC(C)(C(=O)Nc1ccc(Oc2ccc(F)c(Cl)c2)nc1)S(=O)(=O)c1cccc(OC(F)(F)F)c1]

### Preparation of N-(6-(3-chloro-4-fluorophenoxy)pyridin-3-yl)-2-methyl-2-((3-(trifluoromethoxy)phenyl)sulfonyl)propanamide (88)

2-methyl-2-((3-(trifluoromethoxy)phenyl)sulfonyl)propanoic acid (58a) (100 mg, 0.32 mmol), HATU (167 mg, 0.44 mmol), TEA (167 μL, 1.2 mmol), and 6-(3-chloro-4-fluorophenoxy)pyridin-3-amine (87) (76 mg, 0.32 mmol) were stirred in DCM (2 mL) at room temperature for 16 hours. The reaction was concentrated in vacuo, and the residue purified by reverse phase HPLC to give N-(6-(3-chloro-4-fluorophenoxy)pyridin-3-yl)-2-methyl-2-((3-(trifluoromethoxy)phenyl)sulfonyl)propanamide (88).

### (B) General protocol for BOC amino acids amide coupling exemplified by the synthesis of 2-(1-aminocyclohexyl)-N-(3-methyl-3-((3-(trifluoromethyl)phenyl)sulfonyl) butyl)acetamide hydrochloride (91)

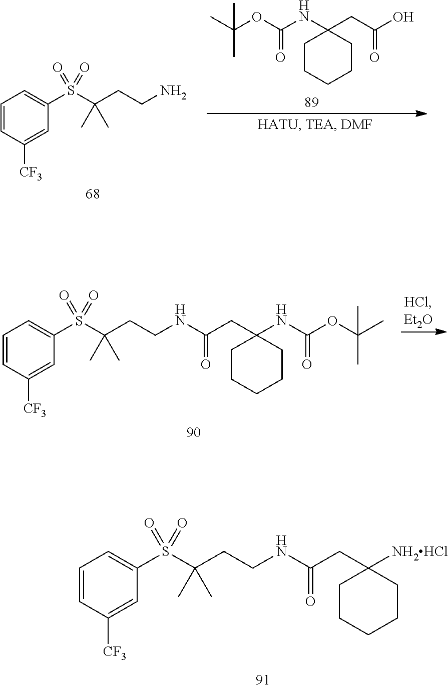

3-Methyl-3-((3-(trifluoromethyl)phenyl)sulfonyl)butan-1-amine (68) (100 mg, 0.34 mmol), HATU (178 mg, 0.48 mmol), TEA (197 μL, 1.41 mmol), and 2-(1-((tert-butoxycarbonyl)amino)cyclohexyl)acetic acid (89) (87 mg, 0.34 mmol) were stirred in DMF (1 mL) at room temperature for 16 hours to afford (90). The reaction was concentrated in vacuo, the residue treated with 2M HCl in Et₂O at room temperature for 5 hours, and quenched with NaHCO₃ saturated solution. The organics were separated, dried, and concentrated in vacuo. The residue was purified by mass directed reverse phase HPLC to give 2-(1-aminocyclohexyl)-N-(3-methyl-3-((3-(trifluoromethyl)phenyl)sulfonyl)butyl)acetamide hydrochloride (91).

### Example 24

### N- and T-Type Channel Blocking Activities

### High-Throughput Ca_{V}2.2/Kᵢᵣ2.3 T-Type Fluorescent Assay

Cells were plated in 384-well, clear-bottom, black-walled, poly-D-lysine coated plates (Becton Dickinson, Franklin Lake, N.J.) 2 days prior to use in the FLIPR assay. 100 μL of cells (1.4×10⁶ cell/mL) containing doxycyline (Sigma-Aldrich, 1.5 μg/mL; to induce channel expression) were added to each well using a Multidrop (Thermo Scientific, Waltham, Mass.) and were maintained in 5% CO₂ incubator at 37° C. On the morning of the assay, cells were transferred to a 5% CO₂ incubator at 29° C.

Cells were washed with a wash buffer containing (in mM): 118 NaCl, 18.4 HEPES, 11.7 D-glucose, 2 CaCl₂, 0.5 MgSO₄, 4.7 KCl, 1.2 KH₂PO₄, pH adjusted to 7.2 with NaOH. 4.4 μM of the fluorescent indicator dye, Fluo-4 (Invitrogen), prepared in pluronic acid (Sigma-Aldrich), was loaded into the wells and incubated for 45 minutes at 29° C. in 5% CO₂. Cells were then rinsed with either a 2 mM KCl closed-state buffer (in mM: 138.5 NaCl, 10 HEPES, 10 D-glucose, 1 CaCl₂, and 2 KCl, with the pH adjusted to 7.4 with NaOH) when performing the closed-state assay or 12.5 mM KCl inactivated-state buffer (in mM: 128 NaCl, 10 HEPES, 10 D-glucose, 1 CaCl₂, and 12.5 mM KCl, with the pH adjusted to 7.4 with NaOH) when performing the inactivated-state assay.

Concentration-dependent response curves were generated from 5 mM stock solutions prepared in DMSO (Sigma-Aldrich) and diluted in either the 2 mM KCl buffer or 12.5 mM KCl buffer and incubated for 20 minutes at 29° C. in 5% CO₂. Calcium entry was evoked with an addition of 130 mM KCl stimulation buffer (in mM: 10.5 NaCl, 10 HEPES, 10 D-glucose, 1 CaCl₂, and 130 KCl, with the pH adjusted to 7.4 with NaOH) for both the closed-state or inactivated-state assay. A change in the Fluo-4 fluorescence signal was assessed using FLIPR^{TETRA}™ instrument (Molecular Devices, Sunnyvale, Calif.) for 3 minutes following the elevation of extracellular KCl using an illumination wavelength of 470-495 nm with emissions recorded at 515-575 nm.

Concentration-dependent response curves were obtained by comparing the fluorescence signal in the presence of compound and fitted with a logistic function (1) to obtain the concentration that inhibited 50% (IC₅₀) of the RLU signal using OriginPro v.7.5 software (OriginLab, Northampton, Mass.).

$$y = [ \begin{matrix}\underline{max - min} \\ 1 + {( \frac{[ drug ]}{{IC}_{50}} )}^{{n}_{H}}\end{matrix} ] + min \qquad ( 1 )$$

To assess the quality of the FLIPR assays the Z-factor (2) was used to quantify the suitability of the assay conditions using the following equation:

$$Z = 1 - \frac{3 {SD}_{sample} + 3 {SD}_{control}}{{mean}_{sample} - {mean}_{control}} \qquad ( 2 )$$

Data are expressed as mean and standard deviation (SD).

### High-Throughput Ca_{V}3.1 T-Type Fluorescent Assay

Cells were plated in 384-well, clear-bottom, black-walled, poly-D-lysine coated plates (Becton Dickinson, Franklin Lake, N.J.) 2 days prior to use in the FLIPR assay. 100 μL of cells (2.0×10⁶ cell/mL) containing doxycyline (Sigma-Aldrich, 1.5 μg/mL; to induce channel expression) were added to each well using a Multidrop (Thermo Scientific, Waltham, Mass.) and were maintained in 5% CO₂ incubator at 37° C. On the morning of the assay, cells were transferred to a 5% CO₂ incubator at 29° C.

Cells were washed with a wash buffer containing (in mM): 118 NaCl, 18.4 HEPES, 11.7 D-glucose, 0.05 CaCl₂, 0.5 MgSO₄, 1 KCl, and 1.2 KH₂PO₄, with the pH adjusted to 7.2 with NaOH. 4.4 μM of the fluorescent indicator dye, Fluo-4 (Invitrogen), prepared in pluronic acid

(Sigma-Aldrich), was loaded into the wells and incubated for 45 minutes at 29° C. in 5% CO₂. Cells were then rinsed with the following low Ca²⁺ buffer (in mM): 0.34 Na₂HPO₄, 4.2 NaHCO₃, 0.44 KH₂PO₄, 0.41 MgSO₄, 0.49 MgCl₂-6H₂O, 20 HEPES, 5.5 D-Glucose, 137 NaCl, 5.3 KCl, and 0.001 CaCl₂, with 0.1% BSA and the pH adjusted to 7.2 with NaOH. Concentration-dependent response curves were generated from 5 mM stock solutions prepared in DMSO (Sigma-Aldrich) and diluted in the buffer containing low Ca²⁺ and incubated for 20 minutes at 29° C. in 5% CO₂. Calcium entry was evoked with an addition of (in mM): 0.34 Na₂HPO₄, 4.2 NaHCO₃, 0.44 KH₂PO₄, 0.41 MgSO₄, 0.49 MgCl₂-6H₂O, 20 HEPES, 5.5 D-Glucose, 137 NaCl, 5.3 KCl, and 6 CaCl₂, with 0.1% BSA and the pH adjusted to 7.2 with NaOH. A change in the Fluo-4 fluorescence signal was assessed using FLIPR^{TETRA}™ instrument (Molecular Devices, Sunnyvale, Calif.) for 3 minutes following the elevation of extracellular KCl using an illumination wavelength of 470-495 nm with emissions recorded at 515-575 nm.

Concentration-dependent response curves were obtained by comparing the fluorescence signal in the presence of compound and fitted with a logistic function (1) to obtain the concentration that inhibited 50% (IC₅₀) of the RLU signal using OriginPro v.7.5 software (OriginLab, Northampton, Mass.).

$$y = [ \begin{matrix}\underline{max - min} \\ 1 + {( \frac{[ drug ]}{{IC}_{50}} )}^{{n}_{H}}\end{matrix} ] + min \qquad ( 1 )$$

To assess the quality of the FLIPR assays the Z-factor (2) was used to quantify the suitability of the assay conditions using the following equation:

$$Z = 1 - \frac{3 {SD}_{sample} + 3 {SD}_{control}}{{mean}_{sample} - {mean}_{control}} \qquad ( 2 )$$

Data are expressed as mean and standard deviation (SD).

### High-Throughput Ca_{V}3.2/Kᵢᵣ2.3 T-Type Fluorescent Assay

Cells were plated in 384-well, clear-bottom, black-walled, poly-D-lysine coated plates (Becton Dickinson, Franklin Lake, N.J.) 2 days prior to use in the FLIPR assay. 1004 of cells (1.2×10⁶ cell/mL) containing doxycyline (Sigma-Aldrich, 1.5 μg/mL; to induce channel expression) were added to each well using a Multidrop (Thermo Scientific, Waltham, Mass.) and were maintained in 5% CO₂ incubator at 37° C. On the morning of the assay, cells were transferred to a 5% CO₂ incubator at 29° C.

Cells were washed with a wash buffer containing (in mM): 118 NaCl, 18.4 HEPES, 11.7 D-glucose, 2 CaCl₂, 0.5 MgSO₄, 4.7 KCl, and 1.2 KH₂PO₄, with the pH adjusted to 7.2 with

NaOH. 4.4 μM of the fluorescent indicator dye Fluo-4 (Invitrogen) prepared in pluronic acid (Sigma-Aldrich) was loaded into the wells and incubated for 45 minutes at 29° C. in 5% CO₂. Cells were then rinsed with either a 2 mM KCl closed-state buffer (in mM: 138.5 NaCl, 10 HEPES, 10 D-glucose, 1 CaCl₂, and 2 KCl, with the pH adjusted to 7.4 with NaOH) when performing the closed-state assay or 7.6 mM KCl inactivated-state buffer (in mM: 130.9 NaCl, 10 HEPES, 10 D-glucose, 1 CaCl₂, and 7.6 mM KCl, with the pH adjusted to 7.4 with NaOH) when performing the inactivated-state assay. Concentration-dependent response curves were generated from 5 mM stock solutions prepared in DMSO (Sigma-Aldrich), diluted in either the 2 mM KCl buffer or 7.6 mM KCl buffer, and incubated for 20 minutes at 29° C. in 5% CO₂. Calcium entry was evoked with an addition of either 12 mM KCl stimulation buffer (in mM: 128.5 NaCl, 10 HEPES, 10 D-glucose, 1 CaCl₂, and 12 KCl, with the pH adjusted to 7.4 with NaOH) or 14.5 mM KCl stimulation buffer (in mM: 126 NaCl, 10 HEPES, 10 D-glucose, 1 CaCl₂, and 14.5 KCl, with the pH adjusted to 7.4 with NaOH) for the closed-state or inactivated-state assay respectively. A change in the Fluo-4 fluorescence signal was assessed using FLIPR^{TETRA}™ instrument (Molecular Devices, Sunnyvale, Calif.) for 3 minutes following the elevation of extracellular KCl using an illumination wavelength of 470-495 nm with emissions recorded at 515-575 nm.

Concentration-dependent response curves were obtained by comparing the fluorescence signal in the presence of compound and fitted with a logistic function (1) to obtain the concentration that inhibited 50% (IC₅₀) of the RLU signal using OriginPro v.7.5 software (OriginLab, Northampton, Mass.).

$$y = [ \begin{matrix}\underline{max - min} \\ 1 + {( \frac{[ drug ]}{{IC}_{50}} )}^{{n}_{H}}\end{matrix} ] + min \qquad ( 1 )$$

To assess the quality of the FLIPR assays the Z-factor (2) was used to quantify the suitability of the assay conditions using the following equation:

$$Z = 1 - \frac{3 {SD}_{sample} + 3 {SD}_{control}}{{mean}_{sample} - {mean}_{control}} \qquad ( 2 )$$

Data are expressed as mean and standard deviation (SD).

Exemplary data obtained according to these procedures are shown in Tables 4 and 5.

TABLE 4
Exemplary Inhibitors of N- and T-Type Calcium Channels

| | FLIPR data for N- and | | | | | |
|---|---|---|---|---|---|---|
| | T-type channels | | | | | |
| | Ca_{V}2.2 | Ca_{V}3.1 | Ca_{V}3.2 | | | |
| No. | (nM) | (nM) | (nM) | Structure | Chemical Name | MW |
| 1 | 760 | 1290 | 620 | 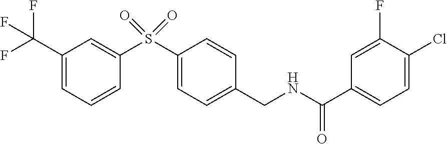 [SMILES: O=C(NCc1ccc(S(=O)(=O)c2cccc(C(F)(F)F)c2)cc1)c1ccc(Cl)c(F)c1] | 4-chloro-3-fluoro-N-(4-((3- (trifluoromethyl)phenyl) sulfonyl)benzyl)benzamide | 471.852 |
| 2 | 950 | 1500 | 850 | 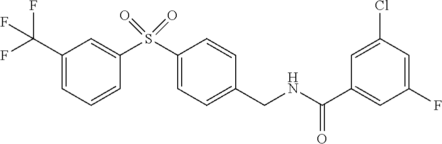 [SMILES: O=C(NCc1ccc(S(=O)(=O)c2cccc(C(F)(F)F)c2)cc1)c1cc(F)cc(Cl)c1] | 3-chloro-5-fluoro-N-(4-((3- (trifluoromethyl)phenyl) sulfonyl)benzyl)benzamide | 471.852 |
| 3 | 510 | 1340 | 650 | 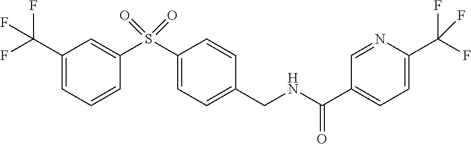 [SMILES: O=C(NCc1ccc(S(=O)(=O)c2cccc(C(F)(F)F)c2)cc1)c1ccc(C(F)(F)F)nc1] | 6-(trifluoromethy)-N-(4- ((3- (trifluoromethyl)phenyl) sulfonyl)benzyl) nicotinamide | 488.403 |
| 4 | 450 | 1480 | 380 | 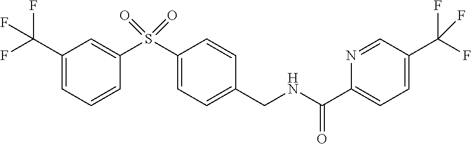 [SMILES: O=C(NCc1ccc(S(=O)(=O)c2cccc(C(F)(F)F)c2)cc1)c1ccc(C(F)(F)F)cn1] | 5-(trifluoromethy)-N-(4- ((3- (trifluoromethyl)phenyl) sulfonyl)benzyl) picolinamide | 488.403 |
| 5 | 470 | 1350 | 2370 | 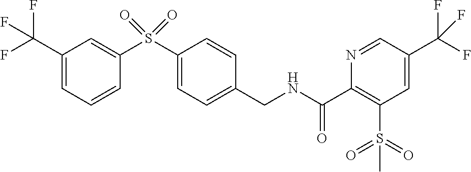 [SMILES: CS(=O)(=O)c1cc(C(F)(F)F)cnc1C(=O)NCc1ccc(S(=O)(=O)c2cccc(C(F)(F)F)c2)cc1] | 3-(methylsulfonyl)-5- (trifluoromethyl)-N-(4-((3- (trifluoromethyl)phenyl) sulfonyl)benzyl) picolinamide | 566.493 |
| 6 | 710 | 2340 | 1510 | 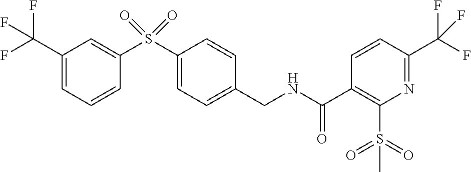 [SMILES: CS(=O)(=O)c1nc(C(F)(F)F)ccc1C(=O)NCc1ccc(S(=O)(=O)c2cccc(C(F)(F)F)c2)cc1] | 2-(methylsulfonyl)-6- (trifluoromethyl)-N-(4-((3- (trifluoromethyl)phenyl) sulfonyl)benzyl) nicotinamide | 566.493 |
| 7 | 390 | 1330 | 510 | 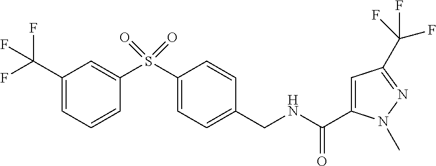 [SMILES: Cn1nc(C(F)(F)F)cc1C(=O)NCc1ccc(S(=O)(=O)c2cccc(C(F)(F)F)c2)cc1] | 1-methyl-3- (trifluoromethyl)-N-(4-((3- (trifluoromethyl)phenyl) sulfonyl)benzyl)-1H- pyrazole-5-carboxamide | 491.407 |
| 8 | 510 | 2140 | 920 | 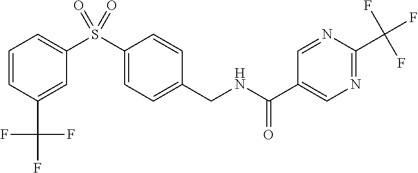 [SMILES: O=C(NCc1ccc(S(=O)(=O)c2cccc(C(F)(F)F)c2)cc1)c1cnc(C(F)(F)F)nc1] | 2-(trifluoromethyl)-N-(4- ((3- (trifluoromethyl)phenyl) sulfonyl)benzyl) pyrimidine-5-carboxamide | 489.391 |
| 9 | 490 | 2850 | 1690 | 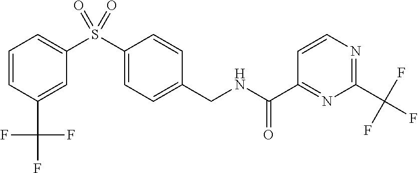 [SMILES: O=C(NCc1ccc(S(=O)(=O)c2cccc(C(F)(F)F)c2)cc1)c1ccnc(C(F)(F)F)n1] | 2-(trifluoromethyl)-N-(4- ((3- (trifluoromethyl)phenyl) sulfonyl)benzyl) pyrimidine-4-carboxamide | 489.391 |
| 10 | 690 | 1970 | 980 | 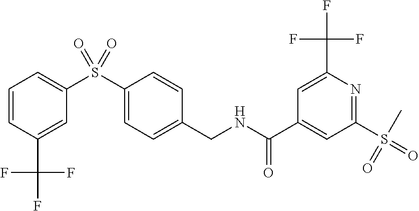 [SMILES: CS(=O)(=O)c1cc(C(=O)NCc2ccc(S(=O)(=O)c3cccc(C(F)(F)F)c3)cc2)cc(C(F)(F)F)n1] | 2-(methylsulfonyl)-6- (trifluoromethyl)-N-(4-((3- (trifluoromethyl)phenyl) sulfonyl)benzyl) isonicotinamide | 566.493 |
| 11 | 410 | 1070 | 590 | 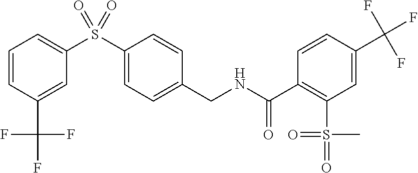 [SMILES: CS(=O)(=O)c1cc(C(F)(F)F)ccc1C(=O)NCc1ccc(S(=O)(=O)c2cccc(C(F)(F)F)c2)cc1] | 2-(methylsulfonyl)-4- (trifluoromethyl)-N-(4-((3- (trifluoromethyl)phenyl) sulfonyl)benzyl)benzamide | 565.505 |
| 12 | 630 | 2290 | 590 | 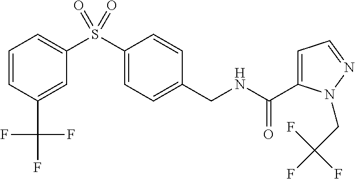 [SMILES: O=C(NCc1ccc(S(=O)(=O)c2cccc(C(F)(F)F)c2)cc1)c1ccnn1CC(F)(F)F] | 1-(2,2,2-trifluoroethyl)-N- (4-((3- (trifluoromethyl)phenyl) sulfonyl)benzyl)-1H- pyrazole-5-carboxamide | 491.407 |
| 13 | 300 | 2020 | 740 | 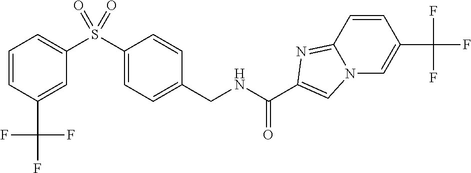 [SMILES: O=C(NCc1ccc(S(=O)(=O)c2cccc(C(F)(F)F)c2)cc1)c1cn2cc(C(F)(F)F)ccc2n1] | 6-(trifluoromethyl)-N-(4- ((3- (trifluoromethyl)phenyl) sulfonyl)benzyl)imidazo [1,2-a]pyridine-2- carboxamide | 527.439 |
| 14 | 850 | 2380 | 430 | 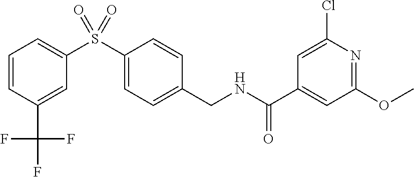 [SMILES: COc1cc(C(=O)NCc2ccc(S(=O)(=O)c3cccc(C(F)(F)F)c3)cc2)cc(Cl)n1] | 2-chloro-6-methoxy-N-(4- ((3- (trifluoromethyl)phenyl) sulfonyl)benzyl) isonicotinamide | 484.876 |
| 15 | 740 | 1390 | 490 | 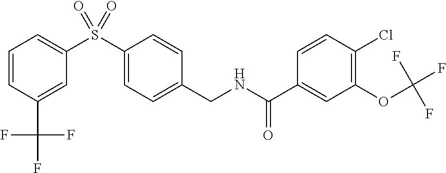 [SMILES: O=C(NCc1ccc(S(=O)(=O)c2cccc(C(F)(F)F)c2)cc1)c1ccc(Cl)c(OC(F)(F)F)c1] | 4-chloro-3- (trifluoromethoxy)-N-(4- ((3- (trifluoromethyl)phenyl) sulfonyl)benzyl)benzamide | 537.859 |
| 16 | 940 | 1430 | 640 | 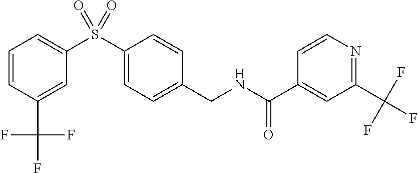 [SMILES: O=C(NCc1ccc(S(=O)(=O)c2cccc(C(F)(F)F)c2)cc1)c1ccnc(C(F)(F)F)c1] | 2-(trifluoromethyl)-N-(4- ((3- (trifluoromethyl)phenyl) sulfonyl)benzyl) isonicotinamide | 488.403 |
| 17 | 140 | 1630 | 940 | 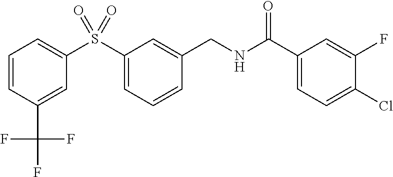 [SMILES: O=C(NCc1cccc(S(=O)(=O)c2cccc(C(F)(F)F)c2)c1)c1ccc(Cl)c(F)c1] | 4-chloro-3-fluoro-N-(3-((3- (trifluoromethyl)phenyl) sulfonyl)benzyl)benzamide | 471.852 |
| 18 | 220 | 1570 | 840 | 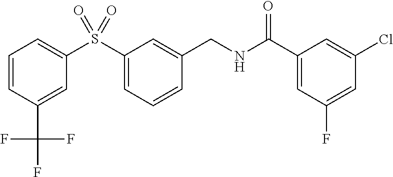 [SMILES: O=C(NCc1cccc(S(=O)(=O)c2cccc(C(F)(F)F)c2)c1)c1cc(F)cc(Cl)c1] | 3-chloro-5-fluoro-N-(3-((3- (trifluoromethyl)phenyl) sulfonyl)benzyl)benzamide | 471.852 |
| 19 | 490 | 2100 | 970 | 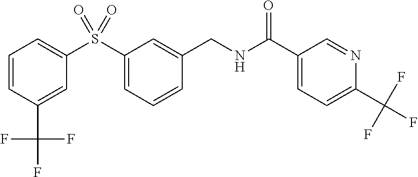 [SMILES: O=C(NCc1cccc(S(=O)(=O)c2cccc(C(F)(F)F)c2)c1)c1ccc(C(F)(F)F)nc1] | 6-(trifluoromethyl)-N-(3- ((3- (trifluoromethyl)phenyl) sulfonyl)benzyl) nicotinamide | 488.403 |
| 20 | 230 | 1810 | 930 | 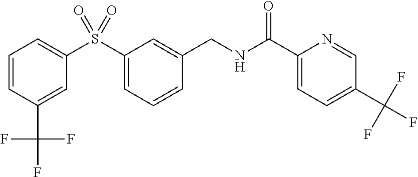 [SMILES: O=C(NCc1cccc(S(=O)(=O)c2cccc(C(F)(F)F)c2)c1)c1ccc(C(F)(F)F)cn1] | 5-(trifluoromethyl)-N-(3- ((3- (trifluoromethyl)phenyl) sulfonyl)benzyl) picolinamide | 488.403 |
| 21 | 760 | 9070 | 2680 | 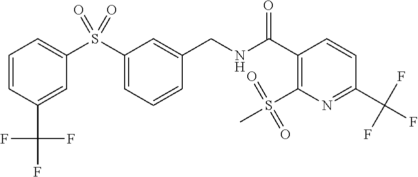 [SMILES: CS(=O)(=O)c1nc(C(F)(F)F)ccc1C(=O)NCc1cccc(S(=O)(=O)c2cccc(C(F)(F)F)c2)c1] | 2-(methylsulfonyl)-6- (trifluoromethyl)-N-(3-((3- (trifluoromethyl)phenyl) sulfonyl)benzyl) nicotinamide | 566.493 |
| 22 | 220 | 3010 | 640 | 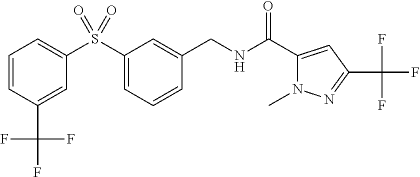 [SMILES: Cn1nc(C(F)(F)F)cc1C(=O)NCc1cccc(S(=O)(=O)c2cccc(C(F)(F)F)c2)c1] | 1-methyl-3- (trifluoromethyl)-N-(3-((3- (trifluoromethyl)phenyl) sulfonyl)benzyl)-1H- pyrazole-5-carboxamide | 491.407 |
| 23 | 490 | 2050 | 570 | 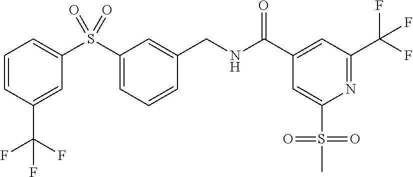 [SMILES: CS(=O)(=O)c1cc(C(=O)NCc2cccc(S(=O)(=O)c3cccc(C(F)(F)F)c3)c2)cc(C(F)(F)F)n1] | 2-(methylsulfonyl)-6- (trifluoromethyl)-N-(3-((3- (trifluoromethyl)phenyl) sulfonyl)benzyl) isonicotinamide | 566.493 |
| 24 | 410 | 3240 | 550 | 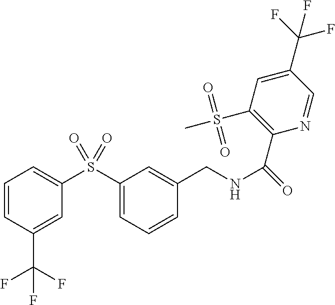 [SMILES: CS(=O)(=O)c1cc(C(F)(F)F)cnc1C(=O)NCc1cccc(S(=O)(=O)c2cccc(C(F)(F)F)c2)c1] | 3-(methylsulfonyl)-5- (trifluoromethyl)-N-(3-((3- (trifluoromethyl)phenyl) sulfonyl)benzyl) picolinamide | 566.493 |
| 25 | 170 | 1580 | 1740 | 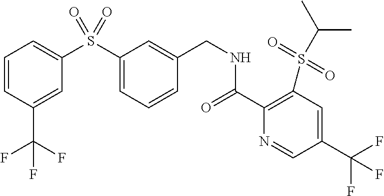 [SMILES: CC(C)S(=O)(=O)c1cc(C(F)(F)F)cnc1C(=O)NCc1cccc(S(=O)(=O)c2cccc(C(F)(F)F)c2)c1] | 3-(isopropylsulfonyl)-5- (trifluoromethyl)-N-(3-((3- (trifluoromethyl)phenyl) sulfonyl)benzyl) picolinamide | 594.546 |
| 26 | 580 | 1880 | 1180 | 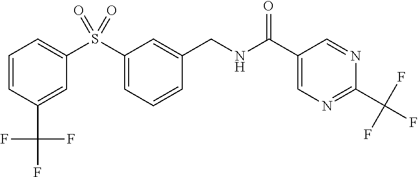 [SMILES: O=C(NCc1cccc(S(=O)(=O)c2cccc(C(F)(F)F)c2)c1)c1cnc(C(F)(F)F)nc1] | 2-(trifluoromethyl)-N-(3- ((3- (trifluoromethyl)phenyl) sulfonyl)benzyl) pyrimidine-5-carboxamide | 489.391 |
| 27 | 310 | 3360 | 2450 | 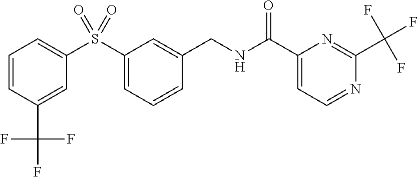 [SMILES: O=C(NCc1cccc(S(=O)(=O)c2cccc(C(F)(F)F)c2)c1)c1ccnc(C(F)(F)F)n1] | 2-(trifluoromethyl)-N-(3- ((3- (trifluoromethyl)phenyl) sulfonyl)benzyl) pyrimidine-4-carboxamide | 489.391 |
| 28 | 190 | 1510 | 730 | 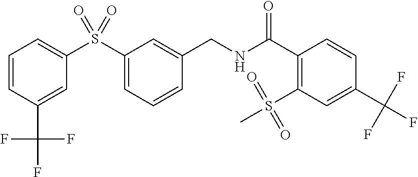 [SMILES: CS(=O)(=O)c1cc(C(F)(F)F)ccc1C(=O)NCc1cccc(S(=O)(=O)c2cccc(C(F)(F)F)c2)c1] | 2-(methylsulfonyl)-4- (trifluoromethyl)-N-(3-((3- (trifluoromethyl)phenyl) sulfonyl)benzyl)benzamide | 565.505 |
| 29 | 340 | 1850 | 600 | 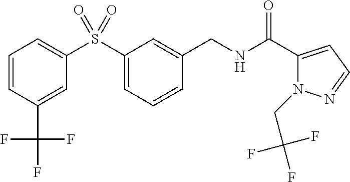 [SMILES: O=C(NCc1cccc(S(=O)(=O)c2cccc(C(F)(F)F)c2)c1)c1ccnn1CC(F)(F)F] | 1-(2,2,2-trifluoroethyl)-N- (3-((3- (trifluoromethyl)phenyl) sulfonyl)benzyl)-1H- pyrazole-5-carboxamide | 491.407 |
| 30 | 310 | 1110 | 510 | 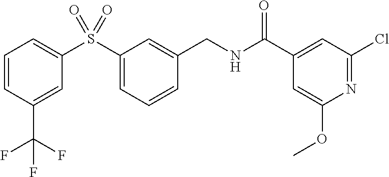 [SMILES: COc1cc(C(=O)NCc2cccc(S(=O)(=O)c3cccc(C(F)(F)F)c3)c2)cc(Cl)n1] | 2-chloro-6-methoxy-N-(3- ((3- (trifluoromethyl)phenyl) sulfonyl)benzyl) isonicotinamide | 484.876 |
| 31 | 390 | 1750 | 730 | 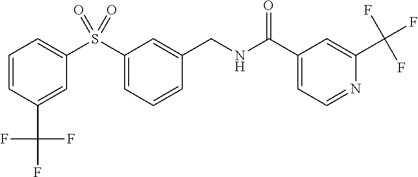 [SMILES: O=C(NCc1cccc(S(=O)(=O)c2cccc(C(F)(F)F)c2)c1)c1ccnc(C(F)(F)F)c1] | 2-(trifluoromethyl)-N-(3- ((3- (trifluoromethyl)phenyl) sulfonyl)benzyl) isonicotinamide | 488.403 |
| 32 | 160 | 1400 | 370 | 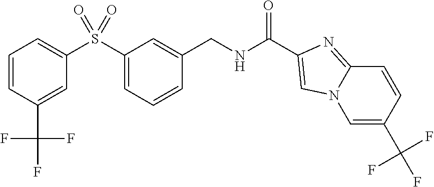 [SMILES: O=C(NCc1cccc(S(=O)(=O)c2cccc(C(F)(F)F)c2)c1)c1cn2cc(C(F)(F)F)ccc2n1] | 6-(trifluoromethyl)-N-(3- ((3- (trifluoromethyl)phenyl) sulfonyl)benzyl)imidazo [1,2-a]pyridine-2- carboxamide | 527.439 |
| 33 | 100 | 920 | 1010 | 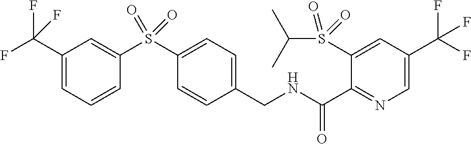 [SMILES: CC(C)S(=O)(=O)c1cc(C(F)(F)F)cnc1C(=O)NCc1ccc(S(=O)(=O)c2cccc(C(F)(F)F)c2)cc1] | 3-(isopropylsulfonyl)-5- (trifluoromethyl)-N-(4-((3- (trifluoromethyl)phenyl) sulfonyl)benzyl) picolinamide | 594.546 |
| 34 | 590 | 1630 | 970 | 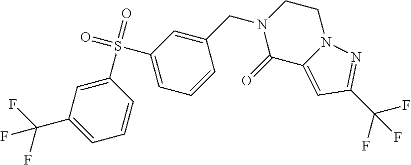 [SMILES: O=C1c2cc(C(F)(F)F)nn2CCN1Cc1cccc(S(=O)(=O)c2cccc(C(F)(F)F)c2)c1] | 2-(trifluoromethyl)-5-(3- ((3- (trifluoromethyl)phenyl) sulfonyl)benzyl)-6,7- dihydropyrazolo[1,5- a]pyrazin-4(5H)-one | 503.418 |
| 35 | 1220 | 1850 | 1740 | 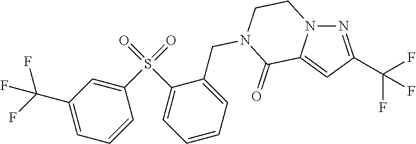 [SMILES: O=C1c2cc(C(F)(F)F)nn2CCN1Cc1ccccc1S(=O)(=O)c1cccc(C(F)(F)F)c1] | 2-(trifluoromethyl)-5-(2- ((3- (trifluoromethyl)phenyl) sulfonyl)benzyl)-6,7- dihydropyrazolo[1,5- a]pyrazin-4(5H)-one | 503.418 |
| 36 | 830 | 410 | 770 | 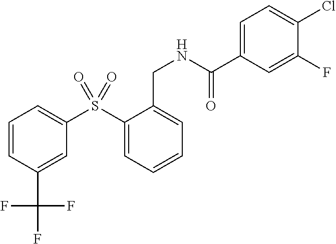 [SMILES: O=C(NCc1ccccc1S(=O)(=O)c1cccc(C(F)(F)F)c1)c1ccc(Cl)c(F)c1] | 4-chloro-3-fluoro-N-(2-((3- (trifluoromethyl)phenyl) sulfonyl)benzyl)benzamide | 471.852 |
| 37 | 680 | 1420 | 1610 | 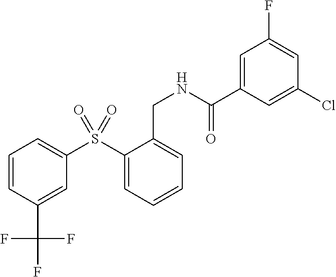 [SMILES: O=C(NCc1ccccc1S(=O)(=O)c1cccc(C(F)(F)F)c1)c1cc(F)cc(Cl)c1] | 3-chloro-5-fluoro-N-(2-((3- (trifluoromethyl)phenyl) sulfonyl)benzyl)benzamide | 471.852 |
| 38 | 1120 | ND | ND | 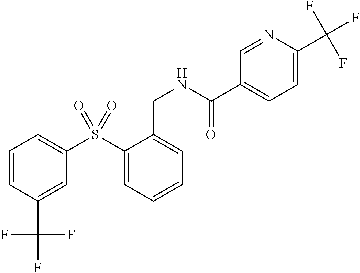 [SMILES: O=C(NCc1ccccc1S(=O)(=O)c1cccc(C(F)(F)F)c1)c1ccc(C(F)(F)F)nc1] | 6-(trifluoromethyl)-N-(2- ((3- (trifluoromethyl)phenyl) sulfonyl)benzyl) nicotinamide | 488.403 |
| 39 | 740 | 1040 | 1250 | 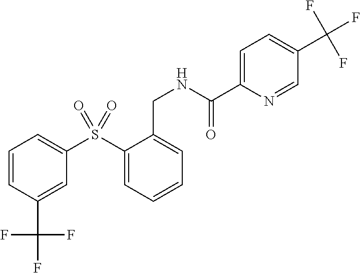 [SMILES: O=C(NCc1ccccc1S(=O)(=O)c1cccc(C(F)(F)F)c1)c1ccc(C(F)(F)F)cn1] | 5-(trifluoromethyl)-N-(2- ((3- (trifluoromethyl)phenyl) sulfonyl)benzyl) picolinamide | 488.403 |
| 40 | 7000 | ND | 10000 | 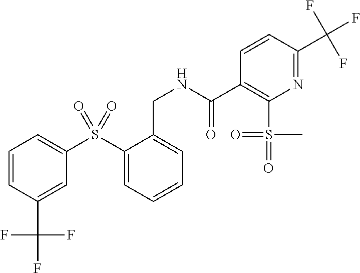 [SMILES: CS(=O)(=O)c1nc(C(F)(F)F)ccc1C(=O)NCc1ccccc1S(=O)(=O)c1cccc(C(F)(F)F)c1] | 2-(methylsulfonyl)-6- (trifluoromethyl)-N-(2-((3- (trifluoromethyl)phenyl) sulfonyl)benzyl) nicotinamide | 566.493 |
| 41 | 380 | 920 | 1390 | 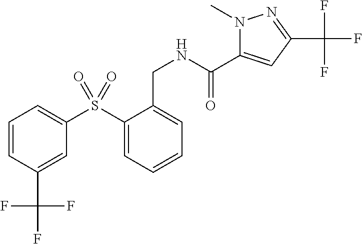 [SMILES: Cn1nc(C(F)(F)F)cc1C(=O)NCc1ccccc1S(=O)(=O)c1cccc(C(F)(F)F)c1] | 1-methyl-3- (trifluoromethyl)-N-(2-((3- (trifluoromethyl)phenyl) sulfonyl)benzyl)-1H- pyrazole-5-carboxamide | 491.407 |
| 42 | 640 | 2890 | 3240 | 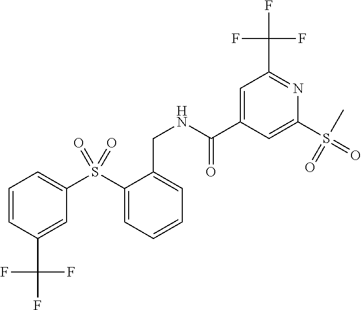 [SMILES: CS(=O)(=O)c1cc(C(=O)NCc2ccccc2S(=O)(=O)c2cccc(C(F)(F)F)c2)cc(C(F)(F)F)n1] | 2-(methylsulfonyl)-6- (trifluoromethyl)-N-(2-((3- (trifluoromethyl)phenyl) sulfonyl)benzyl) isonicotinamide | 566.493 |
| 43 | 670 | 1420 | 3380 | 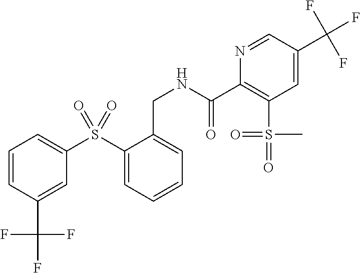 [SMILES: CS(=O)(=O)c1cc(C(F)(F)F)cnc1C(=O)NCc1ccccc1S(=O)(=O)c1cccc(C(F)(F)F)c1] | 3-(methylsulfonyl)-5- (trifluoromethyl)-N-(2-((3- (trifluoromethyl)phenyl) sulfonyl)benzyl) picolinamide | 566.493 |
| 44 | 230 | 720 | 1030 | 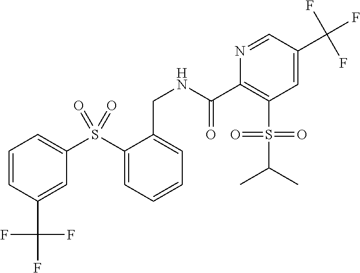 [SMILES: CC(C)S(=O)(=O)c1cc(C(F)(F)F)cnc1C(=O)NCc1ccccc1S(=O)(=O)c1cccc(C(F)(F)F)c1] | 3-(isopropylsulfonyl)-5- (trifluoromethyl)-N-(2-((3- (trifluoromethyl)phenyl) sulfonyl)benzyl) picolinamide | 594.546 |
| 45 | 1110 | ND | ND | 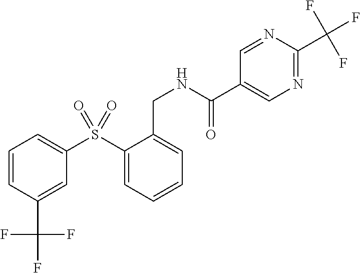 [SMILES: O=C(NCc1ccccc1S(=O)(=O)c1cccc(C(F)(F)F)c1)c1cnc(C(F)(F)F)nc1] | 2-(trifluoromethyl)-N-(2- ((3- (trifluoromethyl)phenyl) sulfonyl)benzyl) pyrimidine-5-carboxamide | 489.391 |
| 46 | 1740 | ND | ND | 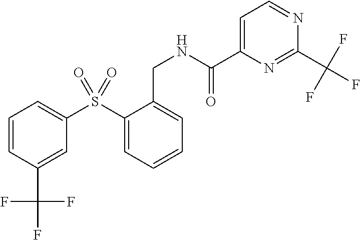 [SMILES: O=C(NCc1ccccc1S(=O)(=O)c1cccc(C(F)(F)F)c1)c1ccnc(C(F)(F)F)n1] | 2-(trifluoromethyl)-N-(2- ((3- (trifluoromethyl)phenyl) sulfonyl)benzyl) pyrimidine-4-carboxamide | 489.391 |
| 47 | 590 | 270 | 2130 | 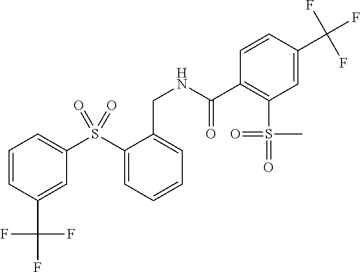 [SMILES: CS(=O)(=O)c1cc(C(F)(F)F)ccc1C(=O)NCc1ccccc1S(=O)(=O)c1cccc(C(F)(F)F)c1] | 2-(methylsulfonyl)-4- (trifluoromethyl)-N-(2-((3- (trifluoromethyl)phenyl) sulfonyl)benzyl)benzamide | 565.505 |
| 48 | 1650 | ND | ND | 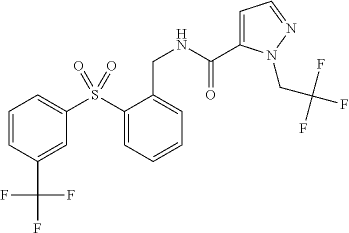 [SMILES: O=C(NCc1ccccc1S(=O)(=O)c1cccc(C(F)(F)F)c1)c1ccnn1CC(F)(F)F] | 1-(2,2,2-trifluoroethyl)-N- (2-((3- (trifluoromethyl)phenyl) sulfonyl)benzyl)-1H- pyrazole-5-carboxamide | 491.407 |
| 49 | 1040 | ND | ND | 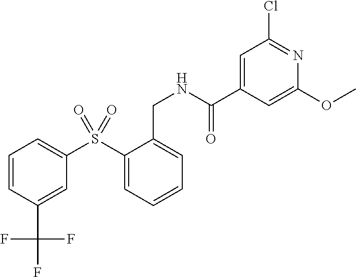 [SMILES: COc1cc(C(=O)NCc2ccccc2S(=O)(=O)c2cccc(C(F)(F)F)c2)cc(Cl)n1] | 2-chloro-6-methoxy-N-(2- ((3- (trifluoromethyl)phenyl) sulfonyl)benzyl) isonicotinamide | 484.876 |
| 50 | 1210 | ND | ND | 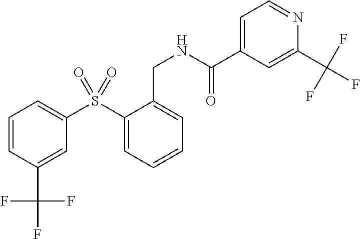 [SMILES: O=C(NCc1ccccc1S(=O)(=O)c1cccc(C(F)(F)F)c1)c1ccnc(C(F)(F)F)c1] | 2-(trifluoromethyl)-N-(2- ((3- (trifluoromethyl)phenyl) sulfonyl)benzyl) isonicotinamide | 488.403 |
| 51 | 1230 | ND | 1050 | 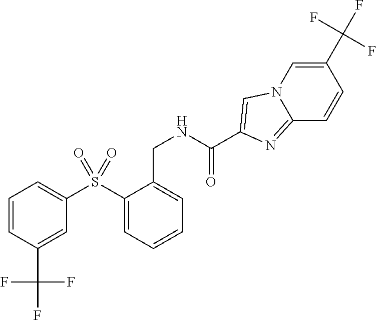 [SMILES: O=C(NCc1ccccc1S(=O)(=O)c1cccc(C(F)(F)F)c1)c1cn2cc(C(F)(F)F)ccc2n1] | 6-(trifluoromethyl)-N-(2- ((3- (trifluoromethyl)phenyl) sulfonyl)benzyl)imidazo [1,2-a]pyridine-2- carboxamide | 527.439 |
| 52 | 740 | 680 | 1300 | 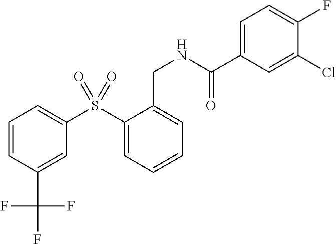 [SMILES: O=C(NCc1ccccc1S(=O)(=O)c1cccc(C(F)(F)F)c1)c1ccc(F)c(Cl)c1] | 3-chloro-4-fluoro-N-(2-((3- (trifluoromethyl)phenyl) sulfonyl)benzyl)benzamide | 471.852 |
| 53 | 340 | 10000 | 4100 | 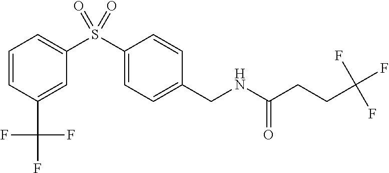 [SMILES: O=C(CCC(F)(F)F)NCc1ccc(S(=O)(=O)c2cccc(C(F)(F)F)c2)cc1] | 4,4,4-trifluoro-N-(4-((3- (trifluoromethyl)phenyl) sulfonyl)benzyl)butanamide | 439.372 |
| 54 | 400 | ND | 6710 | 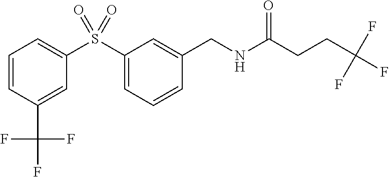 [SMILES: O=C(CCC(F)(F)F)NCc1cccc(S(=O)(=O)c2cccc(C(F)(F)F)c2)c1] | 4,4,4-trifluoro-N-(3-((3- (trifluoromethyl)phenyl) sulfonyl)benzyl)butanamide | 439.372 |
| 55 | 2680 | ND | ND | 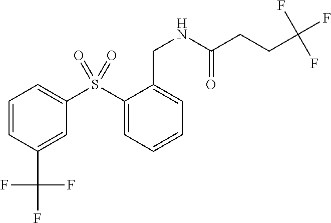 [SMILES: O=C(CCC(F)(F)F)NCc1ccccc1S(=O)(=O)c1cccc(C(F)(F)F)c1] | 4,4,4-trifluoro-N-(2-((3- (trifluoromethyl)phenyl) sulfonyl)benzyl)butanamide | 439.372 |
| 56 | 310 | 10000 | 2660 | 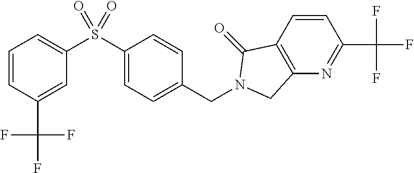 [SMILES: O=C1c2ccc(C(F)(F)F)nc2CN1Cc1ccc(S(=O)(=O)c2cccc(C(F)(F)F)c2)cc1] | 2-(trifluoromethyl)-6-(4- ((3- (trifluoromethyl)phenyl) sulfonyl)benzyl)-6,7- dihydro-5H-pyrrolo[3,4- b]pyridin-5-one | 500.414 |
| 57 | 260 | 6130 | 800 | 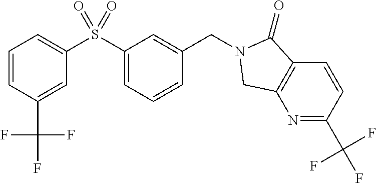 [SMILES: O=C1c2ccc(C(F)(F)F)nc2CN1Cc1cccc(S(=O)(=O)c2cccc(C(F)(F)F)c2)c1] | 2-(trifluoromethyl)-6-(3- ((3- (trifluoromethyl)phenyl) sulfonyl)benzyl)-6,7- dihydro-5H-pyrrolo[3,4- b]pyridin-5-one | 500.414 |
| 58 | 2680 | 10000 | 1480 | 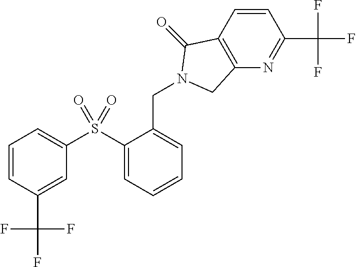 [SMILES: O=C1c2ccc(C(F)(F)F)nc2CN1Cc1ccccc1S(=O)(=O)c1cccc(C(F)(F)F)c1] | 2-(trifluoromethyl)-6-(2- ((3- (trifluoromethyl)phenyl) sulfonyl)benzyl)-6,7- dihydro-5H-pyrrolo[3,4- b]pyridin-5-one | 500.414 |
| 59 | 1000 | 10000 | | 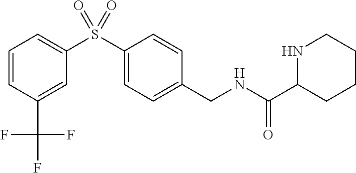 [SMILES: O=C(NCc1ccc(S(=O)(=O)c2cccc(C(F)(F)F)c2)cc1)C1CCCCN1] | N-(4-((3- (trifluoromethyl)phenyl) sulfonyl)benzyl)piperidine- 2-carboxamide | 426.453 |
| 60 | 4230 | ND | | 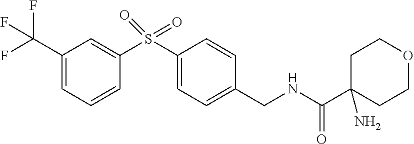 [SMILES: NC1(C(=O)NCc2ccc(S(=O)(=O)c3cccc(C(F)(F)F)c3)cc2)CCOCC1] | 4-amino-N-(4-((3- (trifluoromethyl)phenyl) sulfonyl)benzyl)tetrahydro- 2H-pyran-4-carboxamide | 442.452 |
| 61 | 4680 | ND | | 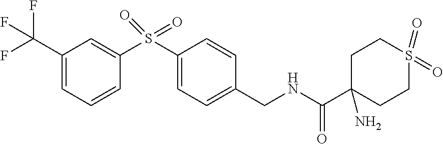 [SMILES: NC1(C(=O)NCc2ccc(S(=O)(=O)c3cccc(C(F)(F)F)c3)cc2)CCS(=O)(=O)CC1] | 4-amino-N-(4-((3- (trifluoromethyl)phenyl) sulfonyl)benzyl)tetrahydro- 2H-thiopyran-4- carboxamide 1,1-dioxide | 490.516 |
| 62 | 620 | 10000 | | 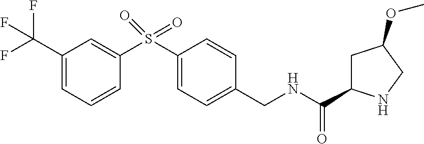 [SMILES: CO[C@H]1CN[C@@H](C(=O)NCc2ccc(S(=O)(=O)c3cccc(C(F)(F)F)c3)cc2)C1] | (2R,4R)-4-methoxy-N-(4- ((3- (trifluoromethyl)phenyl) sulfonyl)benzyl) pyrrolidine-2-carboxamide | 442.452 |
| 63 | 440 | 580 | | 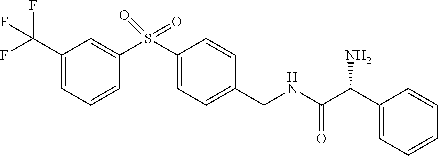 [SMILES: N[C@@H](C(=O)NCc1ccc(S(=O)(=O)c2cccc(C(F)(F)F)c2)cc1)c1ccccc1] | (R)-2-amino-2-phenyl-N- (4-((3- (trifluoromethyl)phenyl) sulfonyl)benzyl)acetamide | 448.458 |
| 64 | 4070 | ND | | 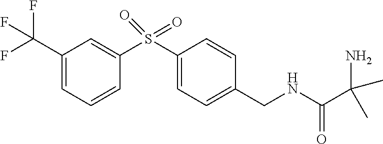 [SMILES: CC(C)(N)C(=O)NCc1ccc(S(=O)(=O)c2cccc(C(F)(F)F)c2)cc1] | 2-amino-2-methyl-N-(4- ((3- (trifluoromethyl)phenyl) sulfonyl)benzyl) propanamide | 400.415 |
| 65 | 5630 | ND | | 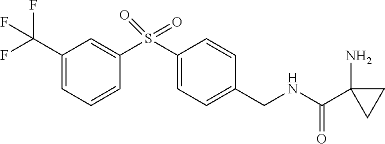 [SMILES: NC1(C(=O)NCc2ccc(S(=O)(=O)c3cccc(C(F)(F)F)c3)cc2)CC1] | 1-amino-N-(4-((3- (trifluoromethyl)phenyl) sulfonyl)benzyl) cyclopropanecarboxamide | 398.399 |
| 66 | 1930 | 10000 | | 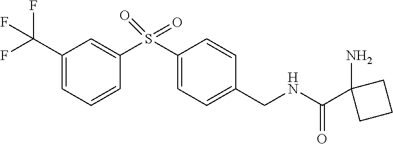 [SMILES: NC1(C(=O)NCc2ccc(S(=O)(=O)c3cccc(C(F)(F)F)c3)cc2)CCC1] | 1-amino-N-(4-((3- (trifluoromethyl)phenyl) sulfonyl)benzyl) cyclobutanecarboxamide | 412.426 |
| 67 | 660 | 3640 | 3740 | 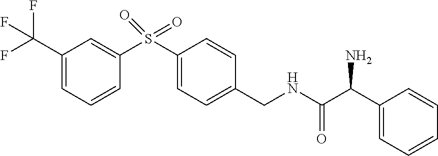 [SMILES: N[C@H](C(=O)NCc1ccc(S(=O)(=O)c2cccc(C(F)(F)F)c2)cc1)c1ccccc1] | (S)-2-amino-2-phenyl-N- (4-((3- (trifluoromethyl)phenyl) sulfonyl)benzyl)acetamide | 448.458 |
| 68 | 2300 | ND | ND | 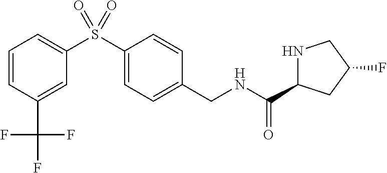 [SMILES: O=C(NCc1ccc(S(=O)(=O)c2cccc(C(F)(F)F)c2)cc1)[C@@H]1C[C@@H](F)CN1] | (2S,4R)-4-fluoro-N-(4-((3- (trifluoromethyl)phenyl) sulfonyl)benzyl) pyrrolidine-2-carboxamide | 430.416 |
| 69 | 9140 | ND | ND | 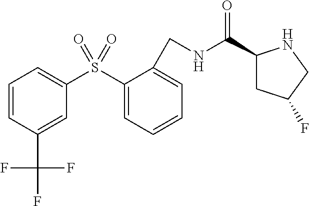 [SMILES: O=C(NCc1ccccc1S(=O)(=O)c1cccc(C(F)(F)F)c1)[C@@H]1C[C@@H](F)CN1] | (2S,4R)-4-fluoro-N-(2-((3- (trifluoromethyl)phenyl) sulfonyl)benzyl) pyrrolidine-2-carboxamide | 430.416 |
| 70 | 4740 | ND | 10000 | 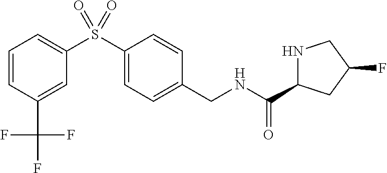 [SMILES: O=C(NCc1ccc(S(=O)(=O)c2cccc(C(F)(F)F)c2)cc1)[C@@H]1C[C@H](F)CN1] | (2S,4S)-4-fluoro-N-(4-((3- (trifluoromethyl)phenyl) sulfonyl)benzyl) pyrrolidine-2-carboxamide | 430.416 |
| 71 | 9490 | ND | ND | 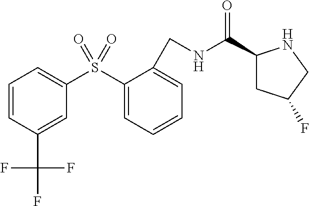 [SMILES: O=C(NCc1ccccc1S(=O)(=O)c1cccc(C(F)(F)F)c1)[C@@H]1C[C@@H](F)CN1] | (2S,4R)-4-hydroxy-N-(2- ((3- (trifluoromethyl)phenyl) sulfonyl)benzyl) pyrrolidine-2-carboxamide | 428.425 |
| 72 | 760 | 8900 | 2320 | 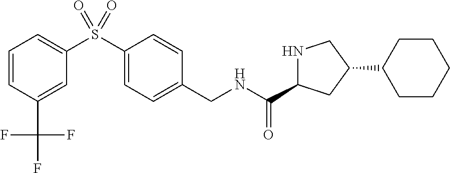 [SMILES: O=C(NCc1ccc(S(=O)(=O)c2cccc(C(F)(F)F)c2)cc1)[C@@H]1C[C@@H](C2CCCCC2)CN1] | (2S,4S)-4-cyclohexyl-N-(4- ((3- (trifluoromethyl)phenyl) sulfonyl)benzyl) pyrrolidine-2-carboxamide | 494.57 |
| 73 | 1920 | 10000 | 10000 | 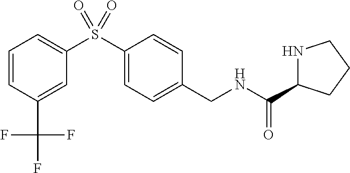 [SMILES: O=C(NCc1ccc(S(=O)(=O)c2cccc(C(F)(F)F)c2)cc1)[C@@H]1CCCN1] | (S)-N-(4-((3- (trifluoromethyl)phenyl) sulfonyl)benzyl) pyrrolidine-2-carboxamide | 412.426 |
| 74 | 2360 | ND | 10000 | 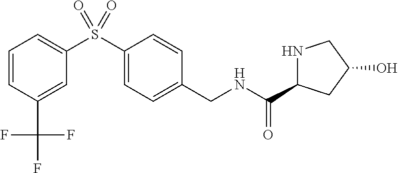 [SMILES: O=C(NCc1ccc(S(=O)(=O)c2cccc(C(F)(F)F)c2)cc1)[C@@H]1C[C@@H](O)CN1] | (2S,4R)-4-hydroxy-N-(4- ((3- (trifluoromethyl)phenyl) sulfonyl)benzyl) pyrrolidine-2-carboxamide | 428.425 |
| 75 | 1060 | 5470 | 1590 | 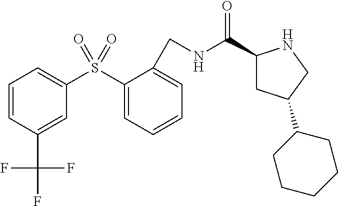 [SMILES: O=C(NCc1ccccc1S(=O)(=O)c1cccc(C(F)(F)F)c1)[C@@H]1C[C@@H](C2CCCCC2)CN1] | (2S,4S)-4-cyclohexyl-N-(2- ((3- (trifluoromethyl)phenyl) sulfonyl)benzyl) pyrrolidine-2-carboxamide | 494.57 |
| 76 | 5540 | ND | 10000 | 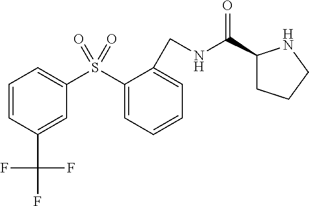 [SMILES: O=C(NCc1ccccc1S(=O)(=O)c1cccc(C(F)(F)F)c1)[C@@H]1CCCN1] | (S)-N-(2-((3- (trifluoromethyl)phenyl) sulfonyl)benzyl) pyrrolidine-2-carboxamide | 412.426 |
| 77 | 690 | 10000 | 10000 | 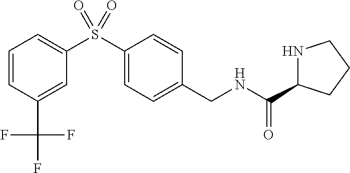 [SMILES: O=C(NCc1ccc(S(=O)(=O)c2cccc(C(F)(F)F)c2)cc1)[C@@H]1CCCN1] | (R)-N-(4-((3- (trifluoromethyl)phenyl) sulfonyl)benzyl) pyrrolidine-2-carboxamide | 412.426 |
| 78 | 3750 | 10000 | 10000 | 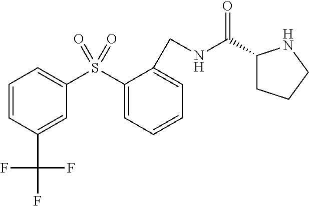 [SMILES: O=C(NCc1ccccc1S(=O)(=O)c1cccc(C(F)(F)F)c1)[C@H]1CCCN1] | (R)-N-(2-((3- (trifluoromethyl)phenyl) sulfonyl)benzyl) pyrrolidine-2-carboxamide | 412.426 |
| 79 | 1450 | 10000 | 8440 | 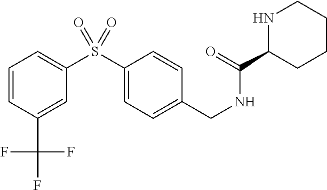 [SMILES: O=C(NCc1ccc(S(=O)(=O)c2cccc(C(F)(F)F)c2)cc1)[C@@H]1CCCCN1] | (S)-N-(4-((3- (trifluoromethyl)phenyl) sulfonyl)benzyl)piperidine- 2-carboxamide | 426.453 |
| 80 | 770 | 10000 | 8700 | 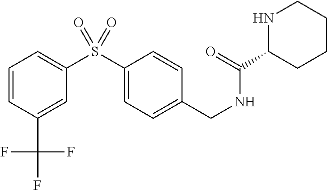 [SMILES: O=C(NCc1ccc(S(=O)(=O)c2cccc(C(F)(F)F)c2)cc1)[C@H]1CCCCN1] | (R)-N-(4-((3- (trifluoromethyl)phenyl) sulfonyl)benzyl)piperidine- 2-carboxamide | 426.453 |
| 81 | 680 | 8240 | 4560 | 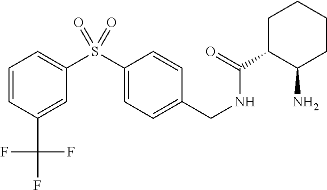 [SMILES: N[C@@H]1CCCC[C@H]1C(=O)NCc1ccc(S(=O)(=O)c2cccc(C(F)(F)F)c2)cc1] | (1R,2R)-2-amino-N-(4-((3- (trifluoromethyl)phenyl) sulfonyl)benzyl) cyclohexanecarboxamide | 440.479 |
| 82 | 970 | ND | 10000 | 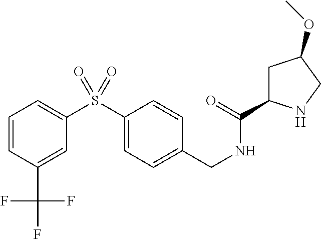 [SMILES: CO[C@H]1CN[C@@H](C(=O)NCc2ccc(S(=O)(=O)c3cccc(C(F)(F)F)c3)cc2)C1] | (2R,4R)-4-methoxy-N-(4- ((3- (trifluoromethyl)phenyl) sulfonyl)benzyl) pyrrolidine-2-carboxamide | 442.452 |
| 83 | 1310 | ND | 9220 | 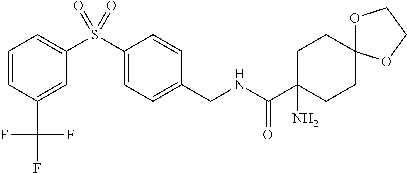 [SMILES: NC1(C(=O)NCc2ccc(S(=O)(=O)c3cccc(C(F)(F)F)c3)cc2)CCC2(CC1)OCCO2] | 8-amino-N-(4-((3- (trifluoromethyl)phenyl) sulfonyl)benzyl)-1,4- dioxaspiro[4.5]decane-8- carboxamide | 498.515 |
| 84 | 2280 | 10000 | 10000 | 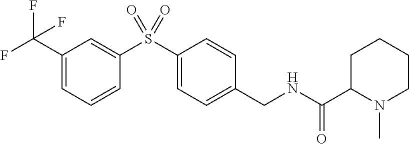 [SMILES: CN1CCCCC1C(=O)NCc1ccc(S(=O)(=O)c2cccc(C(F)(F)F)c2)cc1] | 1-methyl-N-(4-((3- (trifluoromethyl)phenyl) sulfonyl)benzyl)piperidine- 2-carboxamide | 440.479 |
| 85 | 1940 | 10000 | 10000 | 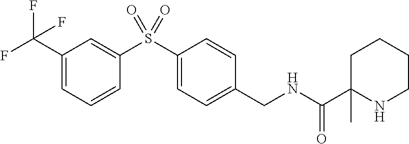 [SMILES: CC1(C(=O)NCc2ccc(S(=O)(=O)c3cccc(C(F)(F)F)c3)cc2)CCCCN1] | 2-methyl-N-(4-((3- (trifluoromethyl)phenyl) sulfonyl)benzyl)piperidine- 2-carboxamide | 440.479 |
| 86 | 310 | 10000 | 5500 | 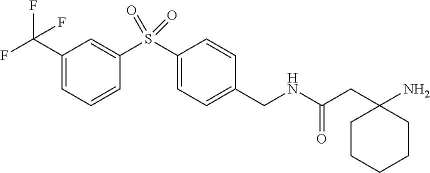 [SMILES: NC1(CC(=O)NCc2ccc(S(=O)(=O)c3cccc(C(F)(F)F)c3)cc2)CCCCC1] | 2-(1-aminocyclohexyl)-N- (4-((3- (trifluoromethyl)phenyl) sulfonyl)benzyl)acetamide | 454.506 |
| 87 | 170 | 1690 | 1260 | 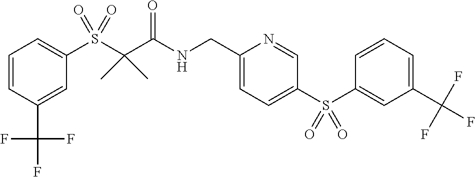 [SMILES: CC(C)(C(=O)NCc1ccc(S(=O)(=O)c2cccc(C(F)(F)F)c2)cn1)S(=O)(=O)c1cccc(C(F)(F)F)c1] | 2-methyl-2-((3- (trifluoromethyl)phenyl) sulfonyl)-N-((5-((3- (trifluoromethyl)phenyl) sulfonyl)pyridin-2- yl)methyl)propanamide | 594.546 |
| 88 | 810 | 900 | 810 | 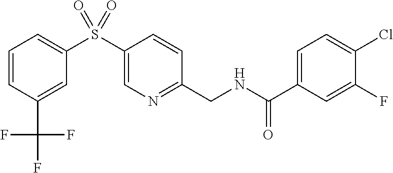 [SMILES: O=C(NCc1ccc(S(=O)(=O)c2cccc(C(F)(F)F)c2)cn1)c1ccc(Cl)c(F)c1] | 4-chloro-3-fluoro-N-((5- ((3- (trifluoromethyl)phenyl) sulfonyl)pyridin-2- yl)methyl)benzamide | 472.84 |
| 89 | 970 | 1580 | 1740 | 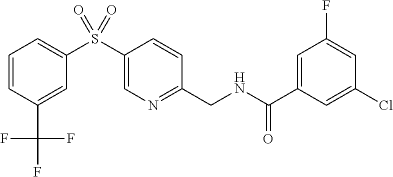 [SMILES: O=C(NCc1ccc(S(=O)(=O)c2cccc(C(F)(F)F)c2)cn1)c1cc(F)cc(Cl)c1] | 3-chloro-5-fluoro-N-((5- ((3- (trifluoromethyl)phenyl) sulfonyl)pyridin-2- yl)methyl)benzamide | 472.84 |
| 90 | 610 | 900 | 2070 | 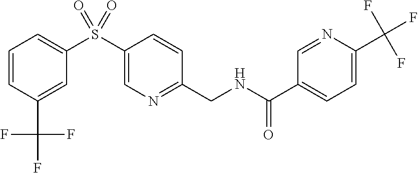 [SMILES: O=C(NCc1ccc(S(=O)(=O)c2cccc(C(F)(F)F)c2)cn1)c1ccc(C(F)(F)F)nc1] | 6-(trifluoromethyl)-N-((5- ((3- (trifluoromethyl)phenyl) sulfonyl)pyridin-2- yl)methyl)nicotinamide | 489.391 |
| 91 | 840 | 1850 | 1290 | 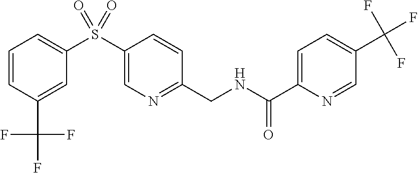 [SMILES: O=C(NCc1ccc(S(=O)(=O)c2cccc(C(F)(F)F)c2)cn1)c1ccc(C(F)(F)F)cn1] | 5-(trifluoromethyl)-N-((5- ((3- (trifluoromethyl)phenyl) sulfonyl)pyridin-2- yl)methyl)picolinamide | 489.391 |
| 92 | 2330 | ND | ND | 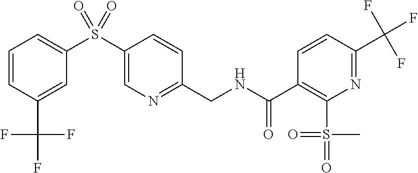 [SMILES: CS(=O)(=O)c1nc(C(F)(F)F)ccc1C(=O)NCc1ccc(S(=O)(=O)c2cccc(C(F)(F)F)c2)cn1] | 2-(methylsulfonyl)-6- (trifluoromethyl)-N-((5-((3- (trifluoromethyl)phenyl) sulfonyl)pyridin-2- yl)methyl)nicotinamide | 567.481 |
| 93 | 570 | 1450 | 740 | 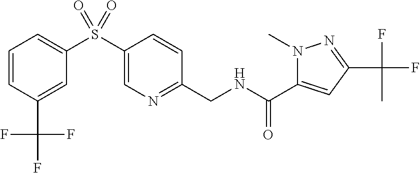 [SMILES: Cn1nc(C(C)(F)F)cc1C(=O)NCc1ccc(S(=O)(=O)c2cccc(C(F)(F)F)c2)cn1] | 1-methyl-3- (trifluoromethyl)-N-((5-((3- (trifluoromethyl)phenyl) sulfonyl)pyridin-2- yl)methyl)-1H-pyrazole-5- carboxamide | 492.395 |
| 94 | 820 | 1630 | 1450 | 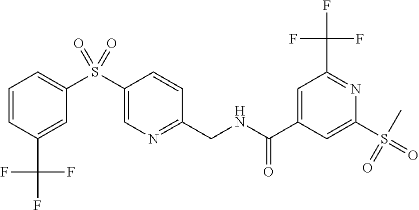 [SMILES: CS(=O)(=O)c1cc(C(=O)NCc2ccc(S(=O)(=O)c3cccc(C(F)(F)F)c3)cn2)cc(C(F)(F)F)n1] | 2-(methylsulfonyl)-6- (trifluoromethyl)-N-((5-((3- (trifluoromethyl)phenyl) sulfonyl)pyridin-2- yl)methyl)isonicotinamide | 567.481 |
| 95 | ND | ND | ND | 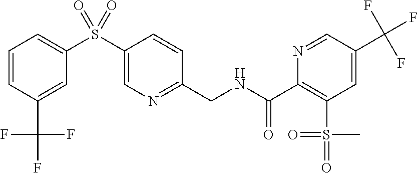 [SMILES: CS(=O)(=O)c1cc(C(F)(F)F)cnc1C(=O)NCc1ccc(S(=O)(=O)c2cccc(C(F)(F)F)c2)cn1] | 3-(methylsulfonyl)-5- (trifluoromethyl)-N-((5-((3- (trifluoromethyl)phenyl) sulfonyl)pyridin-2- yl)methyl)picolinamide | 567.481 |
| 96 | 640 | 1560 | 2140 | 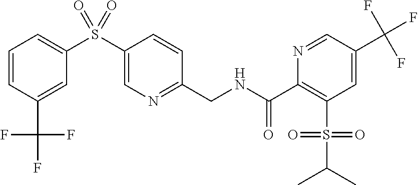 [SMILES: CC(C)S(=O)(=O)c1cc(C(F)(F)F)cnc1C(=O)NCc1ccc(S(=O)(=O)c2cccc(C(F)(F)F)c2)cn1] | 3-(isopropylsulfonyl)-5- (trifluoromethyl)-N-((5-((3- (trifluoromethyl)phenyl) sulfonyl)pyridin-2- yl)methyl)picolinamide | 595.534 |
| 97 | 1930 | ND | 4170 | 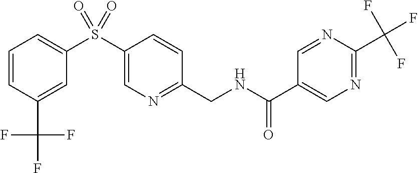 [SMILES: O=C(NCc1ccc(S(=O)(=O)c2cccc(C(F)(F)F)c2)cn1)c1cnc(C(F)(F)F)nc1] | 2-(trifluoromethyl)-N-((5- ((3- (trifluoromethyl)phenyl) sulfonyl)pyridin-2- yl)methyl)pyrimidine-5- carboxamide | 490.379 |
| 98 | 1490 | ND | 9160 | 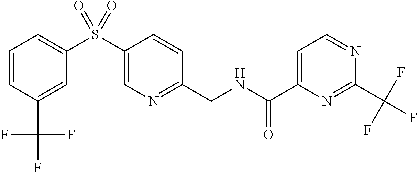 [SMILES: O=C(NCc1ccc(S(=O)(=O)c2cccc(C(F)(F)F)c2)cn1)c1ccnc(C(F)(F)F)n1] | 2-(trifluoromethyl)-N-((5- ((3- (trifluoromethyl)phenyl) sulfonyl)pyridin-2- yl)methyl)pyrimidine-4- carboxamide | 490.379 |
| 99 | 910 | 1270 | 1320 | 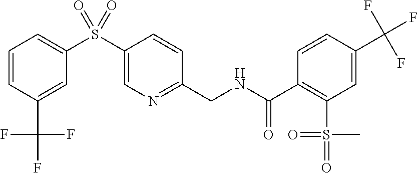 [SMILES: CS(=O)(=O)c1cc(C(F)(F)F)ccc1C(=O)NCc1ccc(S(=O)(=O)c2cccc(C(F)(F)F)c2)cn1] | 2-(methylsulfonyl)-4- (trifluoromethyl)-N-((5-((3- (trifluoromethyl)phenyl) sulfonyl)pyridin-2- yl)methyl)benzamide | 566.493 |
| 100 | 1320 | ND | ND | 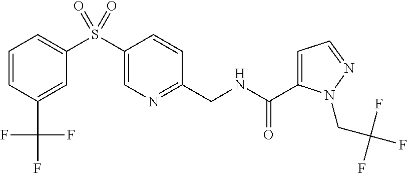 [SMILES: O=C(NCc1ccc(S(=O)(=O)c2cccc(C(F)(F)F)c2)cn1)c1ccnn1CC(F)(F)F] | 1-(2,2,2-trifluoroethyl)-N- ((5-((3- (trifluoromethyl)phenyl) sulfonyl)pyridin-2- yl)methyl)-1H-pyrazole-5- carboxamide | 492.395 |
| 101 | 1460 | ND | ND | 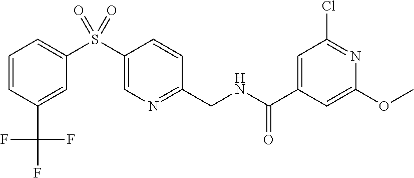 [SMILES: COc1cc(C(=O)NCc2ccc(S(=O)(=O)c3cccc(C(F)(F)F)c3)cn2)cc(Cl)n1] | 2-chloro-6-methoxy-N-((5- ((3- (trifluoromethyl)phenyl) sulfonyl)pyridin-2- yl)methyl)isonicotinamide | 485.864 |
| 102 | 1410 | ND | ND | 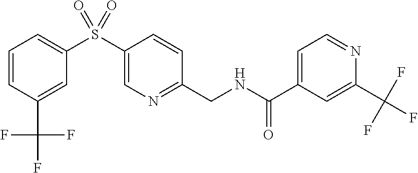 [SMILES: O=C(NCc1ccc(S(=O)(=O)c2cccc(C(F)(F)F)c2)cn1)c1ccnc(C(F)(F)F)c1] | 2-(trifluoromethyl)-N-((5- ((3- (trifluoromethyl)phenyl) sulfonyl)pyridin-2- yl)methyl)isonicotinamide | 489.391 |
| 103 | 830 | 2060 | 1250 | 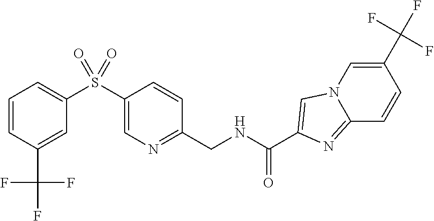 [SMILES: O=C(NCc1ccc(S(=O)(=O)c2cccc(C(F)(F)F)c2)cn1)c1cn2cc(C(F)(F)F)ccc2n1] | 6-(trifluoromethyl)-N-((5- ((3- (trifluoromethyl)phenyl) sulfonyl)pyridin-2- yl)methyl)imidazo[1,2- a]pyridine-2-carboxamide | 528.427 |
| 104 | 600 | 1540 | 880 | 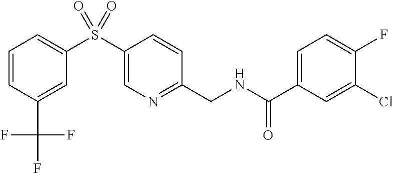 [SMILES: O=C(NCc1ccc(S(=O)(=O)c2cccc(C(F)(F)F)c2)cn1)c1ccc(F)c(Cl)c1] | 3-chloro-4-fluoro-N-((5- ((3- (trifluoromethyl)phenyl) sulfonyl)pyridin-2- yl)methyl)benzamide | 472.84 |
| 105 | 2510 | ND | 10000 | 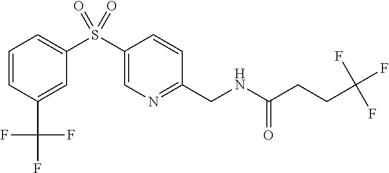 [SMILES: O=C(CCC(F)(F)F)NCc1ccc(S(=O)(=O)c2cccc(C(F)(F)F)c2)cn1] | 4,4,4-trifluoro-N-((5-((3- (trifluoromethyl)phenyl) sulfonyl)pyridin-2- yl)methyl)butanamide | 440.36 |
| 106 | 5850 | 10000 | ND | 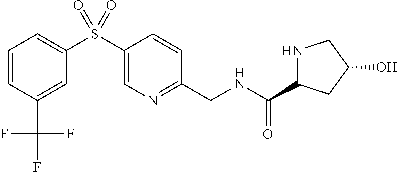 | (2R,4R)-4-hydroxy-N-((5- ((3- (trifluoromethyl)phenyl) sulfonyl)pyridin-2- yl)methyl)pyrrolidine-2- carboxamide | 429.413 |
| 107 | 450 | 6980 | 2090 | 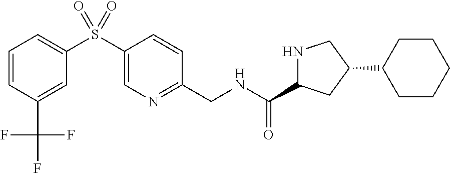 | (2R,4S)-4-cyclohexyl-N- ((5-((3- (trifluoromethyl)phenyl) sulfonyl)pyridin-2- yl)methyl)pyrrolidine-2- carboxamide | 495.558 |
| 108 | 2600 | 10000 | 10000 | 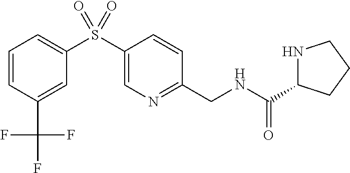 [SMILES: O=C(NCc1ccc(S(=O)(=O)c2cccc(C(F)(F)F)c2)cn1)[C@H]1CCCN1] | (R)-N-((5-((3- (trifluoromethyl)phenyl) sulfonyl)pyridin-2- yl)methyl)pyrrolidine-2- carboxamide | 413.414 |
| 109 | 820 | 6530 | 3970 | 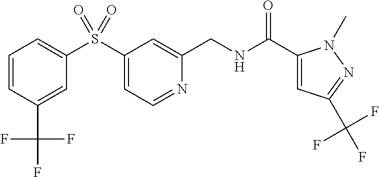 [SMILES: Cn1nc(C(F)(F)F)cc1C(=O)NCc1cc(S(=O)(=O)c2cccc(C(F)(F)F)c2)ccn1] | 1-methyl-3- (trifluoromethyl)-N-((4-((3- (trifluoromethyl)phenyl) sulfonyl)pyridin-2- yl)methyl)-1H-pyrazole-5- carboxamide | 492.395 |
| 110 | 2210 | ND | ND | 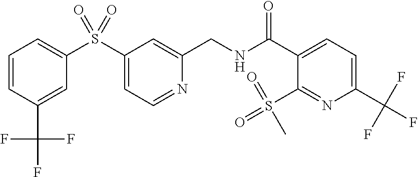 [SMILES: CS(=O)(=O)c1nc(C(F)(F)F)ccc1C(=O)NCc1cc(S(=O)(=O)c2cccc(C(F)(F)F)c2)ccn1] | 2-(methylsulfonyl)-6- (trifluoromethyl)-N-((4-((3- (trifluoromethyl)phenyl) sulfonyl)pyridin-2- yl)methyl)nicotinamide | 567.481 |
| 111 | ND | ND | ND | 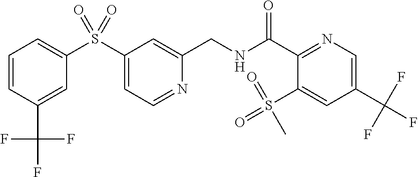 [SMILES: CS(=O)(=O)c1cc(C(F)(F)F)cnc1C(=O)NCc1cc(S(=O)(=O)c2cccc(C(F)(F)F)c2)ccn1] | 3-(methylsulfonyl)-5- (trifluoromethyl)-N-((4-((3- (trifluoromethyl)phenyl) sulfonyl)pyridin-2- yl)methyl)picolinamide | 567.481 |
| 112 | 1030 | ND | ND | 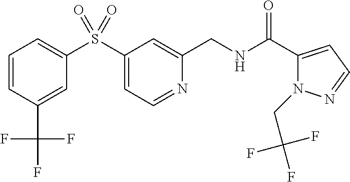 [SMILES: O=C(NCc1cc(S(=O)(=O)c2cccc(C(F)(F)F)c2)ccn1)c1ccnn1CC(F)(F)F] | 1-(2,2,2-trifluoroethyl)-N- ((4-((3- (trifluoromethyl)phenyl) sulfonyl)pyridin-2- yl)methyl)-1H-pyrazole-5- carboxamide | 492.395 |
| 113 | 1390 | ND | ND | 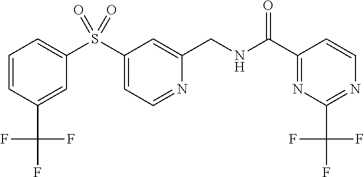 [SMILES: O=C(NCc1cc(S(=O)(=O)c2cccc(C(F)(F)F)c2)ccn1)c1ccnc(C(F)(F)F)n1] | 2-(trifluoromethyl)-N-((4- ((3- (trifluoromethyl)phenyl) sulfonyl)pyridin-2- yl)methyl)pyrimidine-4- carboxamide | 490.379 |
| 114 | 3320 | ND | ND | 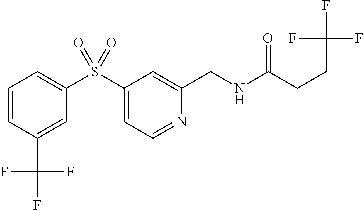 [SMILES: O=C(CCC(F)(F)F)NCc1cc(S(=O)(=O)c2cccc(C(F)(F)F)c2)ccn1] | 4,4,4-trifluoro-N-((4-((3- (trifluoromethyl)phenyl) sulfonyl)pyridin-2- yl)methyl)butanamide | 440.36 |
| 115 | ND | ND | ND | 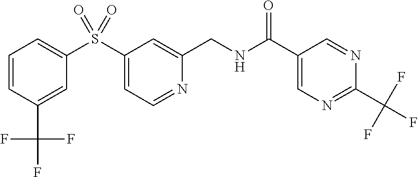 [SMILES: O=C(NCc1cc(S(=O)(=O)c2cccc(C(F)(F)F)c2)ccn1)c1cnc(C(F)(F)F)nc1] | 2-(trifluoromethyl)-N-((4- ((3- (trifluoromethyl)phenyl) sulfonyl)pyridin-2- yl)methyl)pyrimidine-5- carboxamide | 490.379 |
| 116 | 1170 | ND | ND | 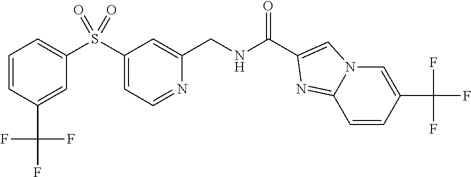 [SMILES: O=C(NCc1cc(S(=O)(=O)c2cccc(C(F)(F)F)c2)ccn1)c1cn2cc(C(F)(F)F)ccc2n1] | 6-(trifluoromethyl)-N-((4- ((3- (trifluoromethyl)phenyl) sulfonyl)pyridin-2- yl)methyl)imidazo[1,2- a]pyridine-2-carboxamide | 528.427 |
| 117 | 7300 | ND | 10000 | 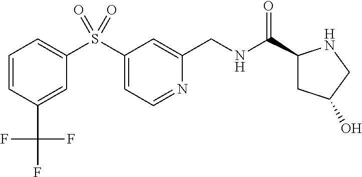 [SMILES: O=C(NCc1cc(S(=O)(=O)c2cccc(C(F)(F)F)c2)ccn1)[C@@H]1C[C@@H](O)CN1] | (2S,4R)-4-hydroxy-4-((4- ((3- (trifluoromethyl)phenyl) sulfonyl)pyridin-2- yl)methyl)pyrrolidine-2- carboxamide | 429.413 |
| 118 | 760 | 4760 | 2400 | 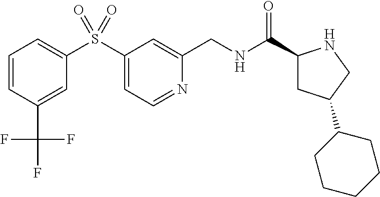 [SMILES: O=C(NCc1cc(S(=O)(=O)c2cccc(C(F)(F)F)c2)ccn1)[C@@H]1C[C@@H](C2CCCCC2)CN1] | (2S,4S)-4-cyclohexyl-N- ((4-((3- (trifluoromethyl)phenyl) sulfonyl)pyridin-2- yl)methyl)pyrrolidine-2- carboxamide | 495.558 |

TABLE 5
Exemplary Inhibitors of N-Type Calcium Channels`

| | FLIPR data for N- and | | | | | |
|---|---|---|---|---|---|---|
| | T-type channels | | | | | |
| | Ca_{V}2.2 | Ca_{V}3.1 | Ca_{V}3.2 | | | |
| No. | (nM) | (nM) | (nM) | Chemical Structure | Compound Name | MW |
| 119 | 200 | 5800 | 4310 | 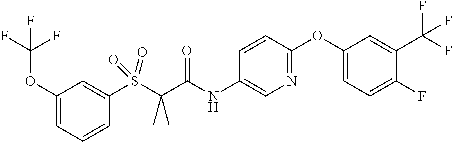 [SMILES: CC(C)(C(=O)Nc1ccc(Oc2ccc(F)c(C(F)(F)F)c2)nc1)S(=O)(=O)c1cccc(OC(F)(F)F)c1] | N-(6-(4-fluoro-3- (trifluoromethyl)phenoxy) pyridin-3-yl)-2-methyl-2-((3- (trifluoromethoxy)phenyl) sulfonyl)propanamide | 566.445 |
| 120 | 1020 | 5690 | 10000 | 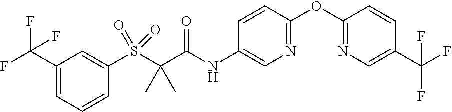 [SMILES: CC(C)(C(=O)Nc1ccc(Oc2ccc(C(F)(F)F)cn2)nc1)S(=O)(=O)c1cccc(C(F)(F)F)c1] | 2-methyl-2-((3- (trifluoromethyl)phenyl) sulfonyl)-N-(6-((5- (trifluoromethyl)pyridin-2- yl)oxy)pyridin-3- yl)propanamide | 533.443 |
| 121 | 660 | 4900 | 1150 | 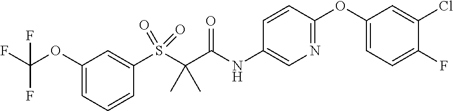 [SMILES: CC(C)(C(=O)Nc1ccc(Oc2ccc(F)c(Cl)c2)nc1)S(=O)(=O)c1cccc(OC(F)(F)F)c1] | N-(6-(3-chloro-4- fluorophenoxy)pyridin-3-yl)- 2-methyl-2-((3- (trifluoromethoxy)phenyl) sulfonyl)propanamide | 532.892 |
| 122 | 700 | 4890 | 1640 | 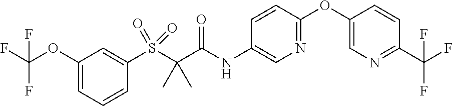 [SMILES: CC(C)(C(=O)Nc1ccc(Oc2ccc(C(F)(F)F)nc2)nc1)S(=O)(=O)c1cccc(OC(F)(F)F)c1] | 2-methyl-2-((3- (trifluoromethoxy)phenyl) sulfonyl)-N-(6-((6- (trifluoromethyl)pyridin-3- yl)oxy)pyridin-3- yl)propanamide | 549.443 |
| 123 | 920 | 4990 | 3010 | 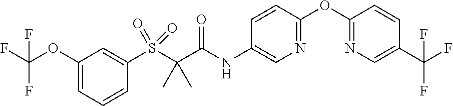 [SMILES: CC(C)(C(=O)Nc1ccc(Oc2ccc(C(F)(F)F)cn2)nc1)S(=O)(=O)c1cccc(OC(F)(F)F)c1] | 2-methyl-2-((3- (trifluoromethoxy)phenyl) sulfonyl)-N-(6-((5- (trifluoromethyl)pyridin-2- yl)oxy)pyridin-3- yl)propanamide | 549.443 |
| 124 | 750 | 4250 | 5620 | 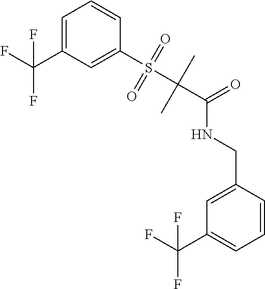 [SMILES: CC(C)(C(=O)NCc1cccc(C(F)(F)F)c1)S(=O)(=O)c1cccc(C(F)(F)F)c1] | 2-methyl-N-(3- (trifluoromethyl)benzyl)-2- ((3-(trifluoromethyl)phenyl) sulfonyl)propanamide | 453.399 |
| 125 | 10000 | ND | ND | 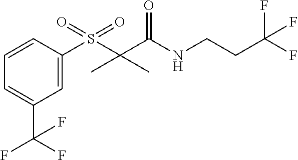 [SMILES: CC(C)(C(=O)NCCC(F)(F)F)S(=O)(=O)c1cccc(C(F)(F)F)c1] | 2-methyl-2-((3- (trifluoromethyl)phenyl) sulfonyl)-N-(3,3,3- trifluoropropyl)propanamide | 391.329 |
| 126 | 4310 | 10000 | 10000 | 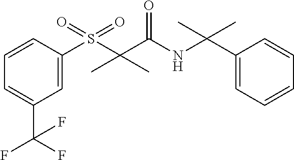 [SMILES: CC(C)(NC(=O)C(C)(C)S(=O)(=O)c1cccc(C(F)(F)F)c1)c1ccccc1] | 2-methyl-N-(2-phenylpropan- 2-yl)-2-((3- (trifluoromethyl)phenyl) sulfonyl)propanamide | 413.454 |
| 127 | 1390 | 5460 | 2960 | 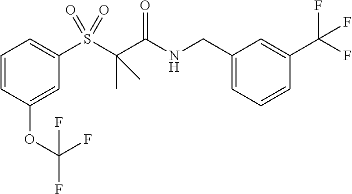 [SMILES: CC(C)(C(=O)NCc1cccc(C(F)(F)F)c1)S(=O)(=O)c1cccc(OC(F)(F)F)c1] | 2-methyl-2-((3- (trifluoromethyl)phenyl) sulfonyl)-N-(3- (trifluoromethyl)benzyl) propanamide | 469.398 |
| 128 | 1200 | 4020 | 3490 | 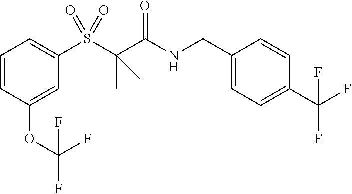 [SMILES: CC(C)(C(=O)NCc1ccc(C(F)(F)F)cc1)S(=O)(=O)c1cccc(OC(F)(F)F)c1] | 2-methyl-2-((3- (trifluoromethyl)phenyl) sulfonyl)-N-(4- (trifluoromethyl)benzyl) propanamide | 469.398 |
| 129 | 10000 | ND | ND | 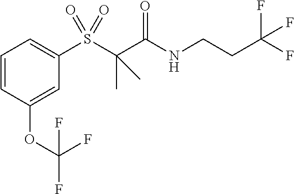 [SMILES: CC(C)(C(=O)NCCC(F)(F)F)S(=O)(=O)c1cccc(OC(F)(F)F)c1] | 2-methyl-2-((3- (trifluoromethyl)phenyl) sulfonyl)-N-(3,3,3- trifluoropropyl)propanamide | 407.329 |
| 130 | 420 | 10000 | 6380 | 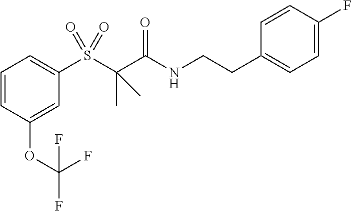 [SMILES: CC(C)(C(=O)NCCc1ccc(F)cc1)S(=O)(=O)c1cccc(OC(F)(F)F)c1] | N-(4-fluorophenethyl)-2- methyl-2-((3- (trifluoromethyl)phenyl) sulfonyl)propanamide | 433.417 |
| 131 | 790 | 6660 | 5210 | 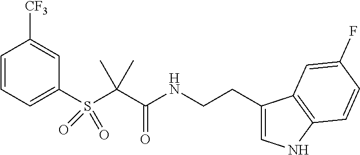 [SMILES: CC(C)(C(=O)NCCc1c[nH]c2ccc(F)cc12)S(=O)(=O)c1cccc(C(F)(F)F)c1] | N-(2-(5-fluoro-1H-indol-3- yl)ethyl)-2-methyl-2-((3- (trifluoromethyl)phenyl) sulfonyl)propanamide | 638.448 |
| 132 | 1210 | 4080 | 3630 | 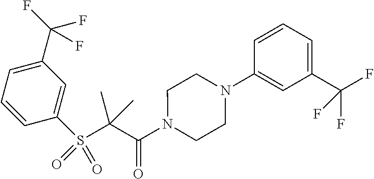 [SMILES: CC(C)(C(=O)N1CCN(c2cccc(C(F)(F)F)c2)CC1)S(=O)(=O)c1cccc(C(F)(F)F)c1] | 2-methyl-1-(4-(3- (trifluoromethyl)phenyl) piperazin-1-yl)-2-((3- (trifluoromethyl)phenyl) sulfonyl)propan-1-one | 508.477 |
| 133 | 1160 | 2160 | 4960 | 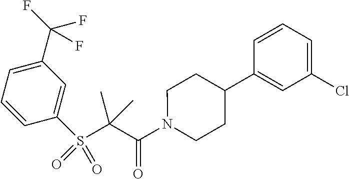 [SMILES: CC(C)(C(=O)N1CCC(c2cccc(Cl)c2)CC1)S(=O)(=O)c1cccc(C(F)(F)F)c1] | 1-(4-(3- chlorophenyl)piperidin-1-yl)- 2-methyl-2-((3- (trifluoromethyl)phenyl) sulfonyl)propan-1-one | 473.936 |
| 134 | 1160 | 8690 | 7580 | 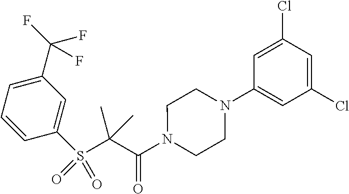 [SMILES: CC(C)(C(=O)N1CCN(c2cc(Cl)cc(Cl)c2)CC1)S(=O)(=O)c1cccc(C(F)(F)F)c1] | 1-(4-(3,5- dichlorophenyl)piperazin-1- yl)-2-methyl-2-((3- (trifluoromethyl)phenyl) sulfonyl)propan-1-one | 509.369 |
| 135 | 1290 | 7950 | 5840 | 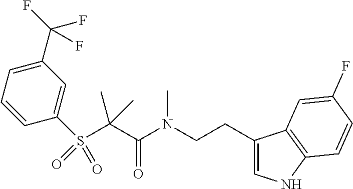 [SMILES: CN(CCc1c[nH]c2ccc(F)cc12)C(=O)C(C)(C)S(=O)(=O)c1cccc(C(F)(F)F)c1] | N-(2-(5-fluoro-1H-indol-3- yl)ethyl)-N,2-dimethyl-2-((3- (trifluoromethyl)phenyl) sulfonyl)propanamide | 470.48 |
| 136 | 390 | 4400 | 2440 | 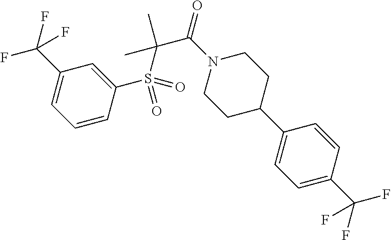 [SMILES: CC(C)(C(=O)N1CCC(c2ccc(C(F)(F)F)cc2)CC1)S(=O)(=O)c1cccc(C(F)(F)F)c1] | 2-methyl-1-(4-(4- (trifluoromethyl)phenyl) piperidin-1-yl)-2-((3- (trifluoromethyl)phenyl) sulfonyl)propan-1-one | 507.489 |
| 137 | 1610 | 4530 | 3000 | 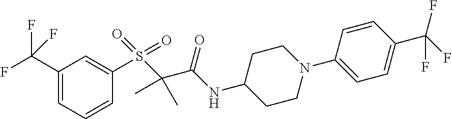 [SMILES: CC(C)(C(=O)NC1CCN(c2ccc(C(F)(F)F)cc2)CC1)S(=O)(=O)c1cccc(C(F)(F)F)c1] | 2-methyl-N-(1-(4- (trifluoromethyl)phenyl) piperidin-4-yl)-2-((3- (trifluoromethyl)phenyl) sulfonyl)propanamide | 522.504 |
| 138 | 1480 | 10000 | 5100 | 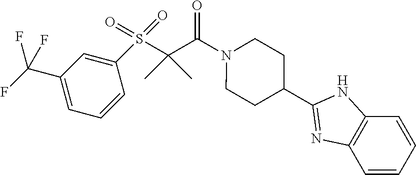 [SMILES: CC(C)(C(=O)N1CCC(c2nc3ccccc3[nH]2)CC1)S(=O)(=O)c1cccc(C(F)(F)F)c1] | 1-(4-(1H-benzol[d]imidazol- 2-yl)piperidin-1-yl)-2-methyl- 2-((3- (trifluoromethyl)phenyl) sulfonyl)propan-1-one | 479.515 |
| 139 | 1220 | ND | 10000 | 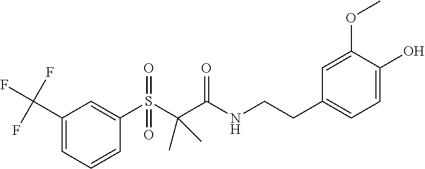 [SMILES: COc1cc(CCNC(=O)C(C)(C)S(=O)(=O)c2cccc(C(F)(F)F)c2)ccc1O] | N-(4-hydroxy-3- methoxyphenethyl)-2-methyl- 2-((3- (trifluoromethyl)phenyl) sulfonyl)propanamide | 445.453 |
| 140 | 410 | 8220 | 5090 | 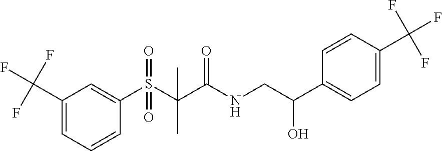 [SMILES: CC(C)(C(=O)NCC(O)c1ccc(C(F)(F)F)cc1)S(=O)(=O)c1cccc(C(F)(F)F)c1] | N-(2-hydroxy-2-(4- (trifluoromethyl)phenyl) ethyl)-2-methyl-2-((3- (trifluoromethyl)phenyl) sulfonyl)propanamide | 483.425 |
| 141 | 1090 | 10000 | 10000 | 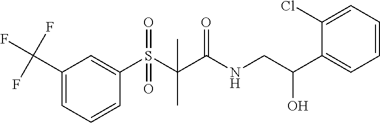 [SMILES: CC(C)(C(=O)NCC(O)c1ccccc1Cl)S(=O)(=O)c1cccc(C(F)(F)F)c1] | N-(2-(2-chlorophenyl)-2- hydroxyethyl)-2-methyl-2- ((3-(trifluoromethyl)phenyl) sulfonyl)propanamide | 449.872 |
| 142 | 440 | 10000 | 10000 | 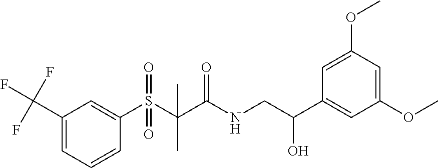 [SMILES: COc1cc(OC)cc(C(O)CNC(=O)C(C)(C)S(=O)(=O)c2cccc(C(F)(F)F)c2)c1] | N-(2-(3,5-dimethoxyphenyl)- 2-hydroxyethyl)-2-methyl-2- ((3-(trifluoromethyl)phenyl) sulfonyl)propanamide | 475.479 |
| 143 | 10000 | ND | 10000 | 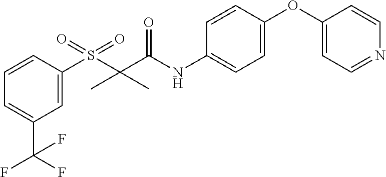 [SMILES: CC(C)(C(=O)Nc1ccc(Oc2ccncc2)cc1)S(=O)(=O)c1cccc(C(F)(F)F)c1] | 2-methyl-N-(4-(pyridin-4- yloxy)phenyl)-2-((3- (trifluoromethyl)phenyl) sulfonyl)propanamide | 464.457 |
| 144 | 360 | 10000 | 3060 | 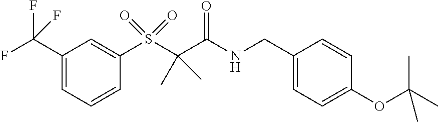 [SMILES: CC(C)(C)Oc1ccc(CNC(=O)C(C)(C)S(=O)(=O)c2cccc(C(F)(F)F)c2)cc1] | N-(4-(tert-butoxy)benzyl)-2- methyl-2-((3- (trifluoromethyl)phenyl) sulfonyl)propanamide | 457.506 |
| 145 | 370 | 5710 | 3050 | 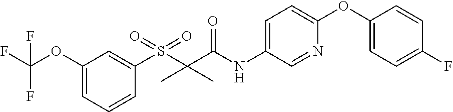 [SMILES: CC(C)(C(=O)Nc1ccc(Oc2ccc(F)cc2)nc1)S(=O)(=O)c1cccc(OC(F)(F)F)c1] | N-(6-(4- fluorophenoxy)pyridin-3-yl)- 2-methyl-2-((3- (trifluoromethyl)phenyl) sulfonyl)propanamide | 498.447 |
| 146 | 440 | 10000 | 7210 | 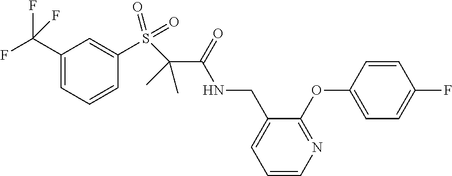 [SMILES: CC(C)(C(=O)NCc1cccnc1Oc1ccc(F)cc1)S(=O)(=O)c1cccc(C(F)(F)F)c1] | N-((2-(4- fluorophenoxy)pyridin-3- yl)methyl)-2-methyl-2-((3- (trifluoromethyl)phenyl) sulfonyl)propanamide | 496.475 |
| 147 | 1390 | 10000 | 3530 | 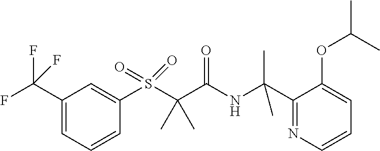 [SMILES: CC(C)Oc1cccnc1C(C)(C)NC(=O)C(C)(C)S(=O)(=O)c1cccc(C(F)(F)F)c1] | N-(2-(3-isopropoxypyridin-2- yl)propan-2-yl)-2-methyl-2- ((3- (trifluoromethyl)phenyl) sulfonyl)propanamide | 472.521 |
| 148 | 10000 | ND | ND | 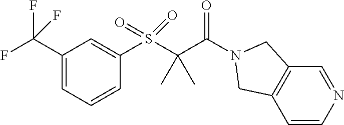 [SMILES: CC(C)(C(=O)N1Cc2ccncc2C1)S(=O)(=O)c1cccc(C(F)(F)F)c1] | 2-methyl-1-(1H-pyrrolo[3,4- c]pyridin-2(3H)-yl)-2-((3- (trifluoromethyl)phenyl) sulfonyl)propan-1-one | 398.399 |
| 149 | 460 | 3850 | 2860 | 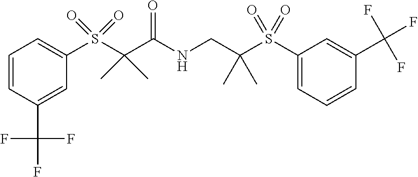 [SMILES: CC(C)(CNC(=O)C(C)(C)S(=O)(=O)c1cccc(C(F)(F)F)c1)S(=O)(=O)c1cccc(C(F)(F)F)c1] | 2-methyl-N-(2-methyl-2-((3- (trifluoromethyl)phenyl) sulfonyl)propyl)-2-((3- (trifluoromethyl)phenyl) sulfonyl)propanamide | 559.542 |
| 150 | 250 | 1770 | 1020 | 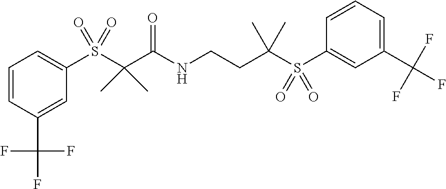 [SMILES: CC(C)(CCNC(=O)C(C)(C)S(=O)(=O)c1cccc(C(F)(F)F)c1)S(=O)(=O)c1cccc(C(F)(F)F)c1] | 2-methyl-N-(3-methyl-3-((3- (trifluoromethyl)phenyl) sulfonyl)butyl)-2-((3- (trifluoromethyl)phenyl) sulfonyl)propanamide | 573.569 |
| 151 | 8230 | ND | ND | 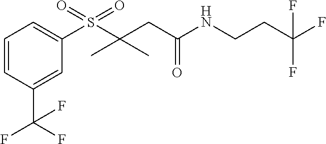 [SMILES: CC(C)(CC(=O)NCCC(F)(F)F)S(=O)(=O)c1cccc(C(F)(F)F)c1] | 3-methyl-3-((3- (trifluoromethyl)phenyl) sulfonyl)-N-(3,3,3- trifluoropropyl)butanamide | 405.356 |
| 152 | 4240 | 10000 | 10000 | 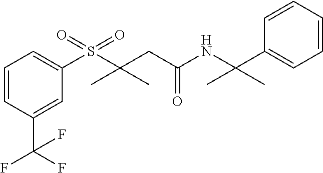 [SMILES: CC(C)(NC(=O)CC(C)(C)S(=O)(=O)c1cccc(C(F)(F)F)c1)c1ccccc1] | 3-methyl-N-(2-phenylpropan- 2-yl)-3-((3- (trifluoromethyl)phenyl) sulfonyl)butanamide | 427.48 |
| 153 | 1220 | ND | ND | 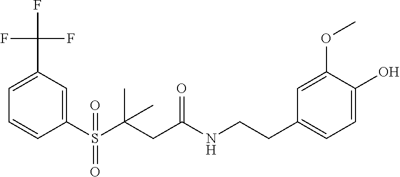 [SMILES: COc1cc(CCNC(=O)CC(C)(C)S(=O)(=O)c2cccc(C(F)(F)F)c2)ccc1O] | N-(4-hydroxy-3- methoxyphenetnyl)-3-methyl- 3-((3- (trifluoromethyl)phenyl) sulfonyl)butanamide | 459.479 |
| 154 | 2230 | 10000 | 10000 | 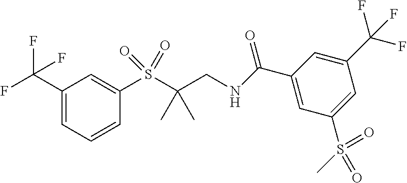 [SMILES: CC(C)(CNC(=O)c1cc(C(F)(F)F)cc(S(C)(=O)=O)c1)S(=O)(=O)c1cccc(C(F)(F)F)c1] | N-(2-methyl-2-((3- (trifluoromethyl)phenyl) sulfonyl)propyl)-2- (methylsulfonyl)-6- (trifluoromethyl) isonicotinamide | 532.477 |
| 155 | 1360 | ND | ND | 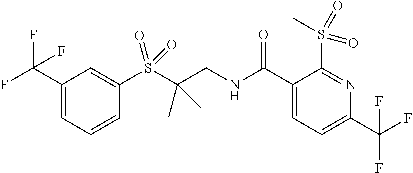 [SMILES: CC(C)(CNC(=O)c1ccc(C(F)(F)F)nc1S(C)(=O)=O)S(=O)(=O)c1cccc(C(F)(F)F)c1] | N-(2-methyl-2-((3- (trifluoromethyl)phenyl) sulfonyl)propyl)-2- (methylsulfonyl)-6- (trifluoromethyl)nicotinamide | 532.477 |
| 156 | 1080 | ND | ND | 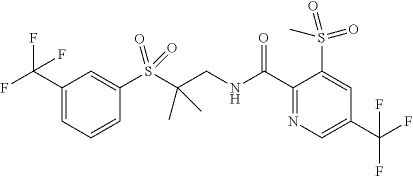 [SMILES: CC(C)(CNC(=O)c1ncc(C(F)(F)F)cc1S(C)(=O)=O)S(=O)(=O)c1cccc(C(F)(F)F)c1] | N-(2-methyl-2-((3- (trifluoromethyl)phenyl) sulfonyl)propyl)-3- (methylsulfonyl)-5- (trifluoromethyl)picolinamide | 532.477 |
| 157 | 5370 | ND | 10000 | 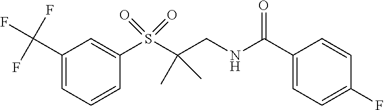 [SMILES: CC(C)(CNC(=O)c1ccc(F)cc1)S(=O)(=O)c1cccc(C(F)(F)F)c1] | 4-fluoro-N-(2-methyl-2-((3- (trifluoromethyl)phenyl) sulfonyl)propyl)benzamide | 403.391 |
| 158 | 2920 | ND | ND | 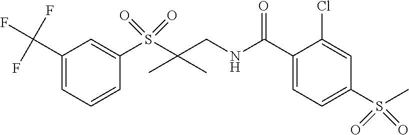 [SMILES: CC(C)(CNC(=O)c1ccc(S(C)(=O)=O)cc1Cl)S(=O)(=O)c1cccc(C(F)(F)F)c1] | 2-chloro-N-(2-methyl-2-((3- (trifluoromethyl)phenyl) sulfonyl)propyl)-4- (methylsulfonyl)benzamide | 497.936 |
| 159 | 1320 | ND | 10000 | 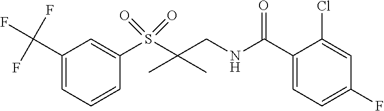 [SMILES: CC(C)(CNC(=O)c1ccc(F)cc1Cl)S(=O)(=O)c1cccc(C(F)(F)F)c1] | 2-chloro-4-fluoro-N-(2- methyl-2-((3- (trifluoromethyl)phenyl) sulfonyl)propyl)benzamide | 437.836 |
| 160 | 1020 | 10000 | 3990 | 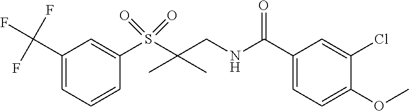 [SMILES: COc1ccc(C(=O)NCC(C)(C)S(=O)(=O)c2cccc(C(F)(F)F)c2)cc1Cl] | 3-chloro-4-methoxy-N-(2- methyl-2-((3- (trifluoromethyl)phenyl) sulfonyl)propyl)benzamide | 449.872 |
| 161 | 1120 | 10000 | 8220 | 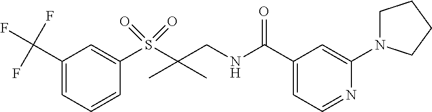 [SMILES: CC(C)(CNC(=O)c1ccnc(N2CCCC2)c1)S(=O)(=O)c1cccc(C(F)(F)F)c1] | N-(2-methyl-2-((3- (trifluoromethyl)phenyl) sulfonyl)propyl)-2- (pyrrolidin-1- yl)isonicotinamide | 455.494 |
| 162 | 2630 | ND | 10000 | 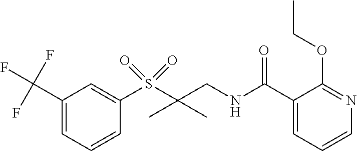 [SMILES: CCOc1ncccc1C(=O)NCC(C)(C)S(=O)(=O)c1cccc(C(F)(F)F)c1] | 2-ethoxy-N-(2-methyl-2-((3- (trifluoromethyl)phenyl) sulfonyl)propyl)nicotinamide | 430.441 |
| 163 | 2250 | ND | 10000 | 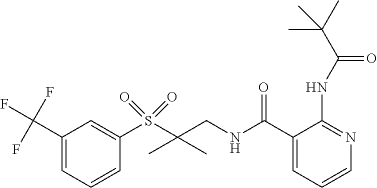 [SMILES: CC(C)(C)C(=O)Nc1ncccc1C(=O)NCC(C)(C)S(=O)(=O)c1cccc(C(F)(F)F)c1] | N-(2-methyl-2-((3- (trifluoromethyl)phenyl) sulfonyl)propyl)-2- pivalamidonicotinamide | 485.52 |
| 164 | 4660 | ND | ND | 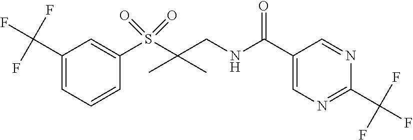 [SMILES: CC(C)(CNC(=O)c1cnc(C(F)(F)F)nc1)S(=O)(=O)c1cccc(C(F)(F)F)c1] | N-(2-methyl-2-((3- (trifluoromethyl)phenyl) sulfonyl)propyl)-2- (trifluoromethyl)pyrimidine- 5-carboxamide | 455.375 |
| 165 | 1530 | 10000 | 6500 | 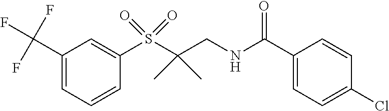 [SMILES: CC(C)(CNC(=O)c1ccc(Cl)cc1)S(=O)(=O)c1cccc(C(F)(F)F)c1] | 4-chloro-N-(2-methyl-2-((3- (trifluoromethyl)phenyl) sulfonyl)propyl)benzamide | 419.846 |
| 166 | 1310 | 10000 | 9680 | 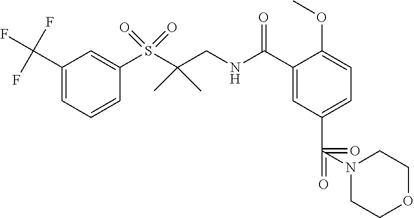 | 2-methoxy-N-(2-methyl-2- ((3-(trifluoromethyl)phenyl) sulfonyl)propyl)-5- (morpholinosulfonyl) benzamide | 564.595 |
| 167 | 1400 | 10000 | 10000 | 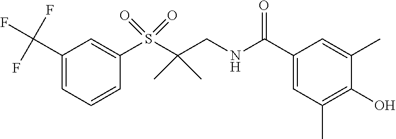 [SMILES: Cc1cc(C(=O)NCC(C)(C)S(=O)(=O)c2cccc(C(F)(F)F)c2)cc(C)c1O] | 4-hydroxy-3,5-dimethyl-N- (2-methyl-2-((3- (trifluoromethyl)phenyl) sulfonyl)propyl)benzamide | 429.453 |
| 168 | 1020 | 10000 | 5160 | 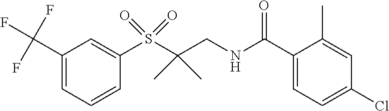 [SMILES: Cc1cc(Cl)ccc1C(=O)NCC(C)(C)S(=O)(=O)c1cccc(C(F)(F)F)c1] | 4-chloro-2-methyl-N-(2- methyl-2-((3- (trifluoromethyl)phenyl) sulfonyl)propyl)benzamide | 433.872 |
| 169 | 1360 | 10000 | 10000 | 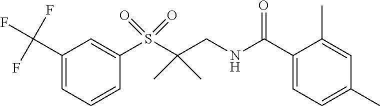 [SMILES: Cc1ccc(C(=O)NCC(C)(C)S(=O)(=O)c2cccc(C(F)(F)F)c2)c(C)c1] | 2,4-dimethyl-N-(2-methyl-2- ((3- (trifluoromethyl)phenyl) sulfonyl)propyl)benzamide | 413.454 |
| 170 | 1860 | 10000 | 7210 | 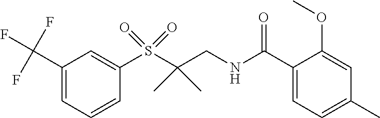 [SMILES: COc1cc(C)ccc1C(=O)NCC(C)(C)S(=O)(=O)c1cccc(C(F)(F)F)c1] | 2-methoxy-4-methyl-N-(2- methyl-2-((3- (trifluoromethyl)phenyl) sulfonyl)propyl)benzamide | 429.453 |
| 171 | 8120 | ND | ND | 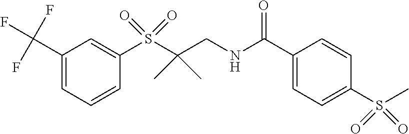 [SMILES: CC(C)(CNC(=O)c1ccc(S(C)(=O)=O)cc1)S(=O)(=O)c1cccc(C(F)(F)F)c1] | N-(2-methyl-2-((3- (trifluoromethyl)phenyl) sulfonyl)propyl)-4- (methylsulfonyl)benzamide | 463.491 |
| 172 | 2170 | ND | 10000 | 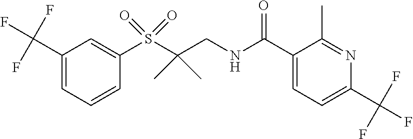 [SMILES: Cc1nc(C(F)(F)F)ccc1C(=O)NCC(C)(C)S(=O)(=O)c1cccc(C(F)(F)F)c1] | 2-methyl-N-(2-methyl-2-((3- (trifluoromethyl)phenyl) sulfonyl)propyl)-6- (trifluoromethyl)nicotinamide | 468.413 |
| 173 | 9060 | 10000 | 10000 | 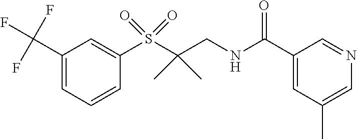 [SMILES: Cc1cncc(C(=O)NCC(C)(C)S(=O)(=O)c2cccc(C(F)(F)F)c2)c1] | 5-methyl-N-(2-methyl-2-((3- (trifluoromethyl)phenyl) sulfonyl)propyl)nicotinamide | 400.415 |
| 174 | 4360 | 10000 | 10000 | 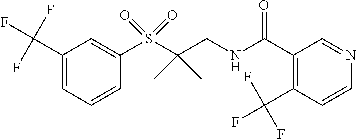 [SMILES: CC(C)(CNC(=O)c1cnccc1C(F)(F)F)S(=O)(=O)c1cccc(C(F)(F)F)c1] | N-(2-methyl-2-((3- (trifluoromethyl)phenyl) sulfonyl)propyl)-4- (trifluoromethyl)nicotinamide | 454.387 |
| 175 | 3190 | 10000 | 5320 | 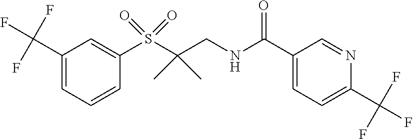 [SMILES: CC(C)(CNC(=O)c1ccc(C(F)(F)F)nc1)S(=O)(=O)c1cccc(C(F)(F)F)c1] | N-(2-methyl-2-((3- (trifluoromethyl)phenyl) sulfonyl)propyl)-6- (trifluoromethyl)nicotinamide | 454.387 |
| 176 | 1310 | 10000 | 5340 | 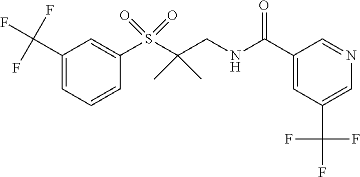 [SMILES: CC(C)(CNC(=O)c1cncc(C(F)(F)F)c1)S(=O)(=O)c1cccc(C(F)(F)F)c1] | N-(2-methyl-2-((3- (trifluoromethyl)phenyl) sulfonyl)propyl)-5- (trifluoromethyl)nicotinamide | 454.387 |
| 177 | 10000 | ND | ND | 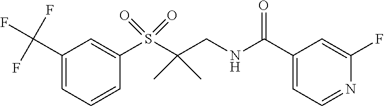 [SMILES: CC(C)(CNC(=O)c1ccnc(F)c1)S(=O)(=O)c1cccc(C(F)(F)F)c1] | 2-fluoro-N-(2-methyl-2-((3- (trifluoromethyl)phenyl) sulfonyl)propyl) isonicotinamide | 404.379 |
| 178 | 1650 | 8960 | 3530 | 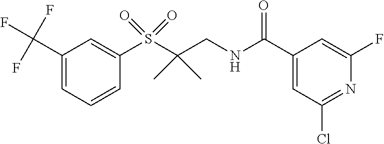 [SMILES: CC(C)(CNC(=O)c1cc(F)nc(Cl)c1)S(=O)(=O)c1cccc(C(F)(F)F)c1] | 2,6-dichloro-N-(2-methyl-2- ((3-(trifluoromethyl)phenyl) sulfonyl)propyl) isonicotinamide | 455.279 |
| 179 | 1240 | 10000 | 10000 | 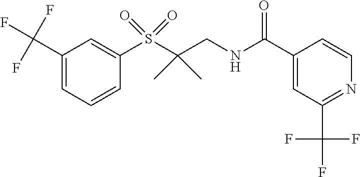 [SMILES: CC(C)(CNC(=O)c1ccnc(C(F)(F)F)c1)S(=O)(=O)c1cccc(C(F)(F)F)c1] | N-(2-methyl-2-((3- (trifluoromethyl)phenyl) sulfonyl)propyl)-2- (trifluoromethyl) isonicotinamide | 454.387 |
| 180 | 3960 | ND | ND | 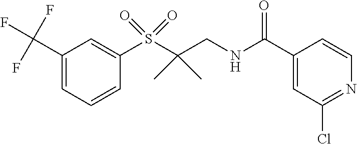 [SMILES: CC(C)(CNC(=O)c1ccnc(Cl)c1)S(=O)(=O)c1cccc(C(F)(F)F)c1] | 2-chloro-N-(2-methyl-2-((3- (trifluoromethyl)phenyl) sulfonyl)propyl) isonicotinamide | 420.834 |
| 181 | ND | ND | ND | 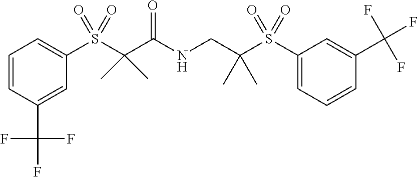 [SMILES: CC(C)(CNC(=O)C(C)(C)S(=O)(=O)c1cccc(C(F)(F)F)c1)S(=O)(=O)c1cccc(C(F)(F)F)c1] | 2-methyl-N-(2-methyl-2-((3- (trifluoromethyl)phenyl) sulfonyl)propyl)-2-((3- (trifluoromethyl)phenyl) sulfonyl)propanamide | 559.542 |
| 182 | 10000 | ND | 10000 | 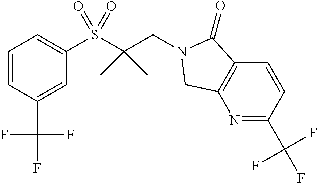 [SMILES: CC(C)(CN1Cc2nc(C(F)(F)F)ccc2C1=O)S(=O)(=O)c1cccc(C(F)(F)F)c1] | 6-(2-methyl-2-((3- (trifluoromethyl)phenyl) sulfonyl)propyl)-2- (trifluoromethyl)-6,7-dihydro- 5H-pyrrolo[3,4-b]pyridin-5- one | 466.397 |
| 183 | 1560 | ND | 10000 | 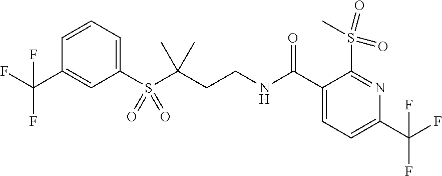 [SMILES: CC(C)(CCNC(=O)c1ccc(C(F)(F)F)nc1S(C)(=O)=O)S(=O)(=O)c1cccc(C(F)(F)F)c1] | N-(3-methyl-3-((3- (trifluoromethyl)phenyl) sulfonyl)butyl)-2- (methylsulfonyl)-6- (trifluoromethyl)nicotinamide | 546.504 |
| 184 | ND | ND | ND | 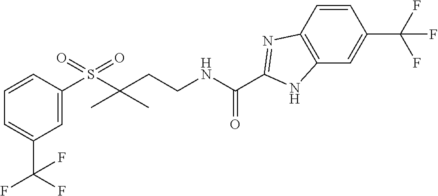 [SMILES: CC(C)(CCNC(=O)c1nc2ccc(C(F)(F)F)cc2[nH]1)S(=O)(=O)c1cccc(C(F)(F)F)c1] | N-(3-methyl-3-((3- (trifluoromethyl)phenyl) sulfonyl)butyl)-6- (trifluoromethyl)-1H- benzo[d]imidazole-2- carboxamide | 507.449 |
| 185 | 380 | ND | 8300 | 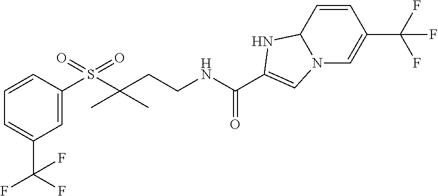 [SMILES: CC(C)(CCNC(=O)C1=CN2C=C(C(F)(F)F)C=CC2N1)S(=O)(=O)c1cccc(C(F)(F)F)c1] | N-(3-methyl-3-((3- (trifluoromethyl)phenyl) sulfonyl)butyl)-6- (trifluoromethyl)imidazo[1,2- a]pyridine-2-carboxamide | 507.449 |
| 186 | ND | ND | ND | 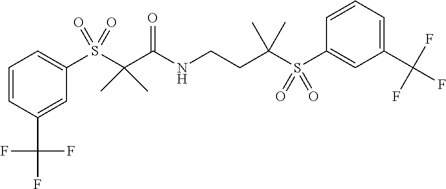 [SMILES: CC(C)(CCNC(=O)C(C)(C)S(=O)(=O)c1cccc(C(F)(F)F)c1)S(=O)(=O)c1cccc(C(F)(F)F)c1] | 2-methyl-N-(3-methyl-3-((3- (trifluoromethyl)phenyl) sulfonyl)butyl)-2-((3- (trifluoromethyl)phenyl) sulfonyl)propanamide | 573.569 |
| 187 | 420 | 5050 | 4310 | 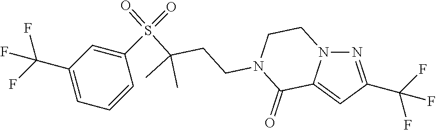 [SMILES: CC(C)(CCN1CCn2nc(C(F)(F)F)cc2C1=O)S(=O)(=O)c1cccc(C(F)(F)F)c1] | 5-(3-methyl-3-((3- (trifluoromethyl)phenyl) sulfonyl)butyl)-2- (trifluoromethyl)-6,7- dihydropyrazolo[1,5- a]pyrazin-4(5H)-one | 483.428 |
| 188 | 420 | 10000 | 5540 | 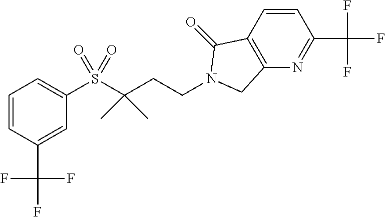 [SMILES: CC(C)(CCN1Cc2nc(C(F)(F)F)ccc2C1=O)S(=O)(=O)c1cccc(C(F)(F)F)c1] | 6-(3-methyl-3-((3- (trifluoromethyl)phenyl) sulfonyl)butyl)-2- (trifluoromethyl)-6,7-dihydro- 5H-pyrolo[3,4-b]pyridine-5- one | 480.424 |
| 189 | 1360 | 10000 | 10000 | 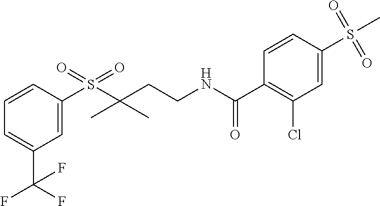 [SMILES: CC(C)(CCNC(=O)c1ccc(S(C)(=O)=O)cc1Cl)S(=O)(=O)c1cccc(C(F)(F)F)c1] | 2-chloro-N-(3-methyl-3-((3- (trifluoromethyl)phenyl) sulfonyl)butyl)-4- (methylsulfonyl)benzamide | 511.963 |
| 190 | 1180 | ND | 10000 | 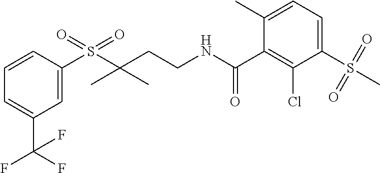 [SMILES: Cc1ccc(S(C)(=O)=O)c(Cl)c1C(=O)NCCC(C)(C)S(=O)(=O)c1cccc(C(F)(F)F)c1] | 2-methyl-N-(3-methyl-3-((3- (trifluoromethyl)phenyl) sulfonyl)butyl)-5- (methylsulfonyl)benzamide | 491.544 |
| 191 | 1420 | ND | 10000 | 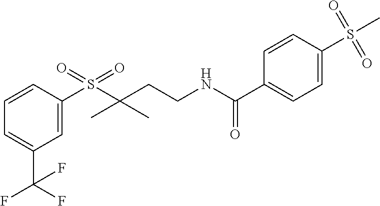 [SMILES: CC(C)(CCNC(=O)c1ccc(S(C)(=O)=O)cc1)S(=O)(=O)c1cccc(C(F)(F)F)c1] | N-(3-methyl-3-((3- (trifluoromethyl)phenyl) sulfonyl)butyl)-4- (methylsulfonyl)benzamide | 477.518 |
| 192 | 3040 | ND | 10000 | 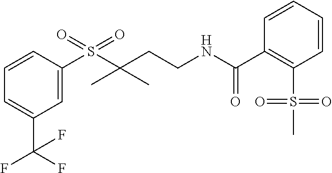 [SMILES: CC(C)(CCNC(=O)c1ccccc1S(C)(=O)=O)S(=O)(=O)c1cccc(C(F)(F)F)c1] | N-(3-methyl-3-((3- (trifluoromethyl)phenyl) sulfonyl)butyl)-2- (methylsulfonyl)benzamide | 477.518 |
| 193 | 1680 | ND | 10000 | 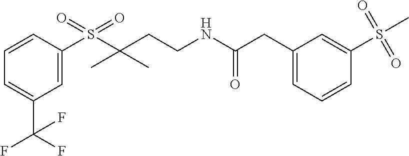 [SMILES: CC(C)(CCNC(=O)Cc1cccc(S(C)(=O)=O)c1)S(=O)(=O)c1cccc(C(F)(F)F)c1] | N-(3-methyl-3-((3- (trifluoromethyl)phenyl) sulfonyl)butyl)-2-(3- (methylsulfonyl)phenyl) acetamide | 491.544 |
| 194 | 1530 | 10000 | 10000 | 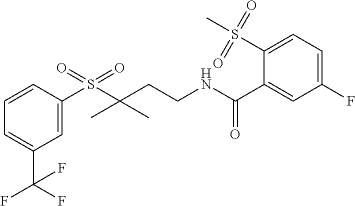 [SMILES: CC(C)(CCNC(=O)c1cc(F)ccc1S(C)(=O)=O)S(=O)(=O)c1cccc(C(F)(F)F)c1] | 5-fluoro-N-(3-methyl-3-((3- (trifluoromethyl)phenyl) sulfonyl)butyl)-2- (methylsulfonyl)benzamide | 495.508 |
| 195 | 3110 | ND | ND | 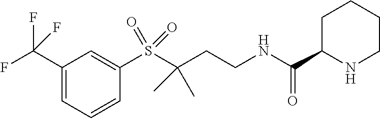 [SMILES: CC(C)(CCNC(=O)[C@H]1CCCCN1)S(=O)(=O)c1cccc(C(F)(F)F)c1] | (R)-N-(3-methyl-3-((3- (trifluoromethyl)phenyl) sulfonyl)butyl)piperidine-2- carboxamide | 406.463 |
| 196 | 3510 | ND | 10000 | 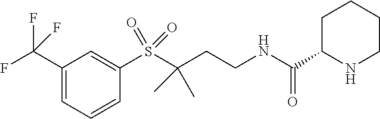 [SMILES: CC(C)(CCNC(=O)[C@@H]1CCCCN1)S(=O)(=O)c1cccc(C(F)(F)F)c1] | (S)-N-(3-methyl-3-((3- (trifluoromethyl)phenyl) sulfonyl)butyl)piperidine-2- carboxamide | 406.463 |
| 197 | 10000 | ND | ND | 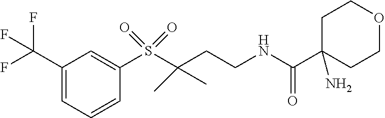 [SMILES: CC(C)(CCNC(=O)C1(N)CCOCC1)S(=O)(=O)c1cccc(C(F)(F)F)c1] | 4-amino-N-(3-methyl-3-((3- (trifluoromethyl)phenyl) sulfonyl)butyl)tetrahydro-2H- pyran-4-carboxamide | 422.462 |
| 198 | 9130 | ND | ND | 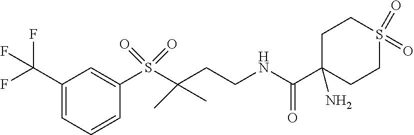 [SMILES: CC(C)(CCNC(=O)C1(N)CCS(=O)(=O)CC1)S(=O)(=O)c1cccc(C(F)(F)F)c1] | 4-amino-N-(3-methyl-3-((3- (trifluoromethyl)phenyl) sulfonyl)butyl)tetrahydro-2H- thiopyran-4-carboxamide 1,1- dioxide | 470.527 |
| 199 | 3310 | ND | ND | 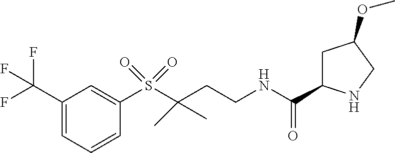 [SMILES: CO[C@H]1CN[C@@H](C(=O)NCCC(C)(C)S(=O)(=O)c2cccc(C(F)(F)F)c2)C1] | (2R,4R)-4-methoxy-N-(3- methyl-3-((3- (trifluoromethyl)phenyl) sulfonyl)butyl)pyrrolidine-2- carboxamide | 422.462 |
| 200 | 1050 | ND | ND | 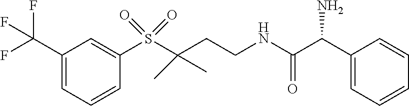 [SMILES: CC(C)(CCNC(=O)[C@H](N)c1ccccc1)S(=O)(=O)c1cccc(C(F)(F)F)c1] | (R)-2-amino-N-(3-methyl-3- ((3- (trifluoromethyl)phenyl) sulfonyl)butyl-2- phenylacetamide | 428.468 |
| 201 | 10000 | ND | ND | 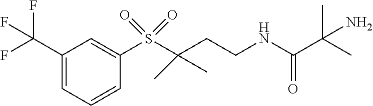 [SMILES: CC(C)(N)C(=O)NCCC(C)(C)S(=O)(=O)c1cccc(C(F)(F)F)c1] | 2-amino-2-methyl-N-(3- methyl-3-((3- (trifluoromethyl)phenyl) sulfonyl)butyl)propanamide | 380.426 |
| 202 | 10000 | ND | ND | 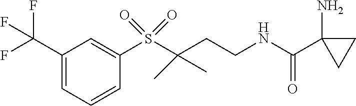 [SMILES: CC(C)(CCNC(=O)C1(N)CC1)S(=O)(=O)c1cccc(C(F)(F)F)c1] | 1-amino-N-(3-methyl-3-((3- (trifluoromethyl)phenyl) sulfonyl)butyl) cyclopropanecarboxamide | 378.41 |
| 203 | 5850 | ND | ND | 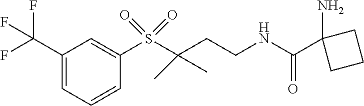 [SMILES: CC(C)(CCNC(=O)C1(N)CCC1)S(=O)(=O)c1cccc(C(F)(F)F)c1] | 1-amino-N-(3-methyl-3-((3- (trifluoromethyl)phenyl) sulfonyl)butyl) cyclobutanecarboxamide | 392.436 |
| 204 | 10250 | ND | ND | 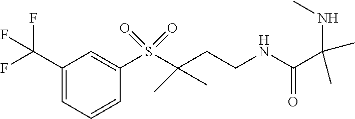 [SMILES: CNC(C)(C)C(=O)NCCC(C)(C)S(=O)(=O)c1cccc(C(F)(F)F)c1] | 2-methyl-N-(3-methyl-3-((3- (trifluoromethyl)phenyl) sulfonyl)butyl)-2- (methylamino)propanamide | 394.452 |
| 205 | 1480 | ND | ND | 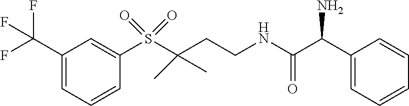 [SMILES: CC(C)(CCNC(=O)[C@@H](N)c1ccccc1)S(=O)(=O)c1cccc(C(F)(F)F)c1] | (S)-2-amino-N-(3-methyl-3- ((3-(trifluoromethyl)phenyl) sulfonyl)butyl)-2- phenylacetamide | 428.468 |
| 206 | 10000 | ND | ND | 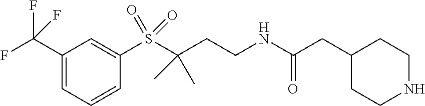 [SMILES: CC(C)(CCNC(=O)CC1CCNCC1)S(=O)(=O)c1cccc(C(F)(F)F)c1] | N-(3-methyl-3-((3- (trifluoromethyl)phenyl) sulfonyl)butyl)-2-(piperidin- 4-yl)acetamide | 420.489 |
| 207 | 1390 | ND | ND | 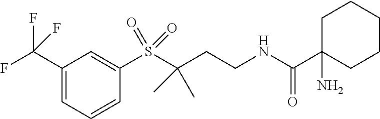 [SMILES: CC(C)(CCNC(=O)C1(N)CCCCC1)S(=O)(=O)c1cccc(C(F)(F)F)c1] | 1-amino-N-(3-methyl-3-((3- (trifluoromethyl)phenyl) sulfonyl)butyl) cyclohexanecarboxamide | 420.489 |
| 208 | 1750 | 10000 | ND | 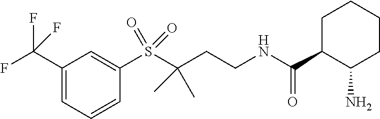 [SMILES: CC(C)(CCNC(=O)[C@H]1CCCC[C@@H]1N)S(=O)(=O)c1cccc(C(F)(F)F)c1] | (1S,2S)-2-amino-N-(3- methyl-3-((3- (trifluoromethyl)phenyl) sulfonyl)butyl) cyclohexanecarboxamide | 420.489 |
| 209 | 1680 | ND | ND | 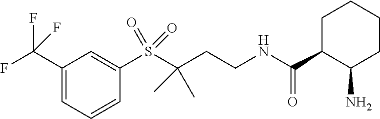 [SMILES: CC(C)(CCNC(=O)[C@H]1CCCC[C@H]1N)S(=O)(=O)c1cccc(C(F)(F)F)c1] | (1S,2R)-2-amino-N-(3- methyl-3-((3- (trifluoromethyl)phenyl) sulfonyl)butyl) cyclohexanecarboxamide | 420.489 |
| 210 | 3320 | ND | ND | 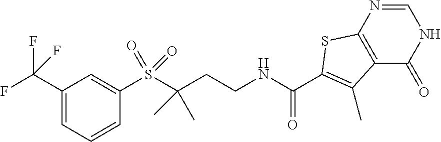 [SMILES: Cc1c(C(=O)NCCC(C)(C)S(=O)(=O)c2cccc(C(F)(F)F)c2)sc2nc[nH]c(=O)c12] | 5-methyl-N-(3-methyl-3-((3- (trifluoromethyl)phenyl) sulfonyl)butyl)-4-oxo-3,4- dihydrothieno[2,3- d]pyrimidine-6-carboxamide | 487.516 |
| 211 | 1100 | ND | ND | 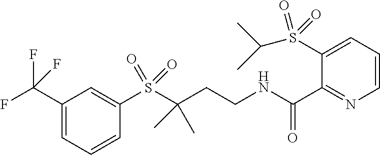 [SMILES: CC(C)S(=O)(=O)c1cccnc1C(=O)NCCC(C)(C)S(=O)(=O)c1cccc(C(F)(F)F)c1] | 3-(isopropylsulfonyl)-N-(3- methyl-3-((3- (trifluoromethyl)phenyl) sulfonyl)butyl)picolinamide | 506.559 |
| 212 | 1440 | ND | ND | 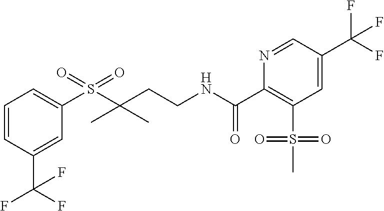 [SMILES: CC(C)(CCNC(=O)c1ncc(C(F)(F)F)cc1S(C)(=O)=O)S(=O)(=O)c1cccc(C(F)(F)F)c1] | N-(3-((3-fluoro-5- (trifluoromethyl)phenyl) sulfonyl)-3-methylbutyl)-3- (methylsulfonyl)-5- (trifluoromethyl)picolinamide | 564.494 |
| 213 | 1120 | ND | ND | 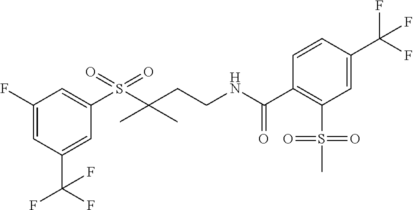 [SMILES: CC(C)(CCNC(=O)c1ccc(C(F)(F)F)cc1S(C)(=O)=O)S(=O)(=O)c1cc(F)cc(C(F)(F)F)c1] | N-(3-((3-fluoro-5- (trifluoromethyl)phenyl) sulfonyl)-3-methylbutyl)-2- (methylsulfonyl)-4- (trifluoromethyl)benzamide | 563.506 |
| 214 | 1310 | ND | ND | 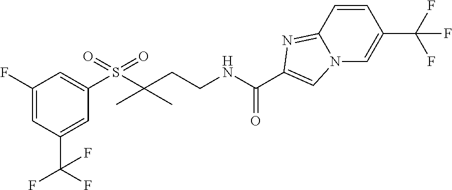 [SMILES: CC(C)(CCNC(=O)c1cn2cc(C(F)(F)F)ccc2n1)S(=O)(=O)c1cc(F)cc(C(F)(F)F)c1] | N-(3-((3-fluoro-5- (trifluoromethyl)phenyl) sulfonyl)-3-methylbutyl)-6- (trifluoromethyl)imidazo[1,2- a]pyridine-2-carboxamide | 525.44 |
| 215 | 3160 | 10000 | 10000 | 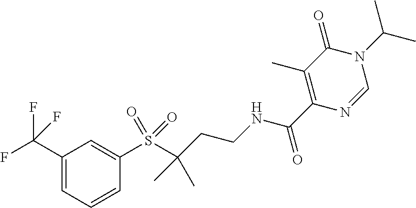 [SMILES: Cc1c(C(=O)NCCC(C)(C)S(=O)(=O)c2cccc(C(F)(F)F)c2)ncn(C(C)C)c1=O] | 1-isopropyl-5-methyl-N-(3- methyl-3-((3- (trifluoromethyl)phenyl) sulfonyl)butyl)-6-oxo-1,6- dihydropyrimidine-4- carboxamide | 473.509 |
| 216 | 8930 | ND | 10000 | 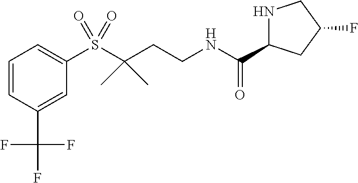 [SMILES: CC(C)(CCNC(=O)[C@@H]1C[C@@H](F)CN1)S(=O)(=O)c1cccc(C(F)(F)F)c1] | (2S,4R)-4-fluoro-N-(3- methyl-3-((3- (trifluoromethyl)phenyl) sulfonyl)butyl)pyrrolidine-2- carboxamide | 410.427 |
| 217 | 8170 | ND | ND | 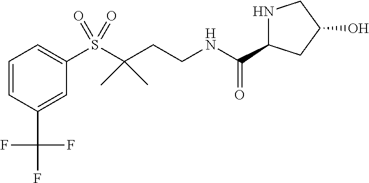 [SMILES: CC(C)(CCNC(=O)[C@@H]1C[C@@H](O)CN1)S(=O)(=O)c1cccc(C(F)(F)F)c1] | (2S,4R)-4-hydroxy-N-(3- methyl-3-((3- (trifluoromethyl)phenyl) sulfonyl)butyl)pyrrolidine-2- carboxamide | 408.436 |
| 218 | 740 | 5640 | 3210 | 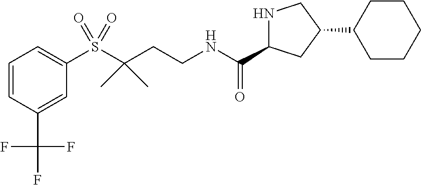 [SMILES: CC(C)(CCNC(=O)[C@@H]1C[C@@H](C2CCCCC2)CN1)S(=O)(=O)c1cccc(C(F)(F)F)c1] | (2S,4S)-4-cyclohexyl-N-(3- methyl-3-((3- (trifluoromethyl)phenyl) sulfonyl)butyl)pyrrolidine-2- carboxamide | 474.58 |
| 219 | 3230 | ND | ND | 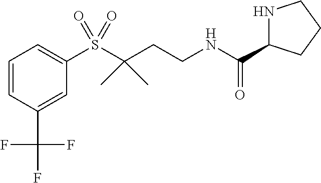 [SMILES: CC(C)(CCNC(=O)[C@@H]1CCCN1)S(=O)(=O)c1cccc(C(F)(F)F)c1] | (S)-N-(3-methyl-3-((3- (trifluoromethyl)phenyl) sulfonyl)butyl)pyrrolidine-2- carboxamide | 392.436 |
| 220 | 3430 | ND | ND | 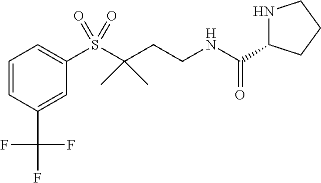 [SMILES: CC(C)(CCNC(=O)[C@H]1CCCN1)S(=O)(=O)c1cccc(C(F)(F)F)c1] | (R)-N-(3-methyl-3-((3- (trifluoromethyl)phenyl) sulfonyl)butyl)pyrrolidine-2- carboxamide | 392.436 |
| 221 | 6100 | 10000 | ND | 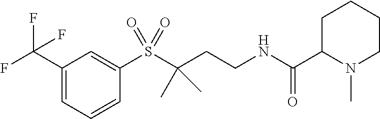 [SMILES: CN1CCCCC1C(=O)NCCC(C)(C)S(=O)(=O)c1cccc(C(F)(F)F)c1] | 1-methyl-N-(3-methyl-3-((3- (trifluoromethyl)phenyl) sulfonyl)butyl)piperidine-2- carboxamide | 420.489 |
| 222 | 3770 | 10000 | 10000 | 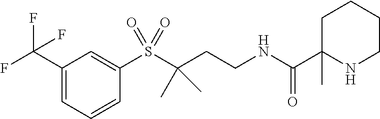 [SMILES: CC1(C(=O)NCCC(C)(C)S(=O)(=O)c2cccc(C(F)(F)F)c2)CCCCN1] | 2-methyl-N-(3-methyl-3-((3- (trifluoromethyl)phenyl) sulfonyl)butyl)piperidine-2- carboxamide | 420.489 |
| 223 | 360 | ND | 10000 | 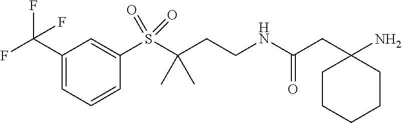 [SMILES: CC(C)(CCNC(=O)CC1(N)CCCCC1)S(=O)(=O)c1cccc(C(F)(F)F)c1] | 2-(1-aminocyclohexyl)-N-(3- methyl-3-((3- (trifluoromethyl)phenyl) sulfonyl)butyl)acetamide | 434.516 |
| 224 | 470 | ND | 10000 | 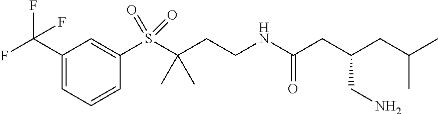 [SMILES: CC(C)C[C@H](CN)CC(=O)NCCC(C)(C)S(=O)(=O)c1cccc(C(F)(F)F)c1] | (S)-3-(aminomethyl)-5- methyl-N-(3-methyl-3-((3- (trifluoromethyl)phenyl) sulfonyl)butyl)hexanamide | 436.53 |
| 225 | 100 | 1410 | 630 | 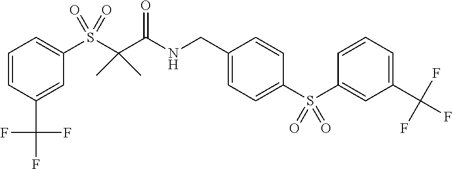 [SMILES: CC(C)(C(=O)NCc1ccc(S(=O)(=O)c2cccc(C(F)(F)F)c2)cc1)S(=O)(=O)c1cccc(C(F)(F)F)c1] | 2-methyl-2-((3- (trifluoromethyl)phenyl) sulfonyl)-N-(4-((3- (trifluoromethyl)phenyl) sulfonyl)benzyl)propanamide | 593.558 |
| 226 | 110 | 1410 | 1200 | 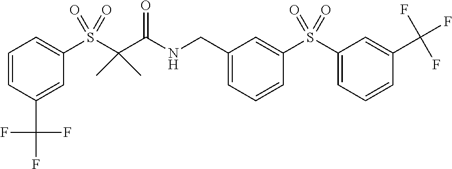 [SMILES: CC(C)(C(=O)NCc1cccc(S(=O)(=O)c2cccc(C(F)(F)F)c2)c1)S(=O)(=O)c1cccc(C(F)(F)F)c1] | 2-methyl-2-((3- (trifluoromethyl)phenyl) sulfonyl)-N-(3-((3- (trifluoromethyl)phenyl) sulfonyl)benzyl)propanamide | 593.558 |
| 227 | 400 | 630 | 350 | 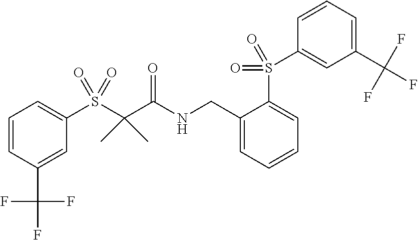 [SMILES: CC(C)(C(=O)NCc1ccccc1S(=O)(=O)c1cccc(C(F)(F)F)c1)S(=O)(=O)c1cccc(C(F)(F)F)c1] | 2-methyl-2-((3- (trifluoromethyl)phenyl) sulfonyl)-N-(2-((3- (trifluoromethyl)phenyl) sulfonyl)benzyl)propanamide | 593.558 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

What is claimed is:

1. A compound having a structure according to the following formula, 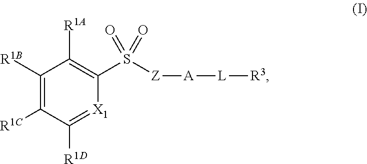
or a pharmaceutically acceptable salt, solvate, or prodrug thereof, or a stereoisomer thereof, wherein
X¹ is N or CR^{1E};
each of R^{1A}, R^{1B}, R^{1C}, R^{1D}, and R^{1E} is selected, independently, from H, OH, halogen, optionally substituted C1-C6 alkyl, optionally substituted C2-C6 alkenyl, optionally substituted C2-C6 alkynyl, and optionally substituted C1-C6 alkoxy;
Z is —(CR^{Z1}R^{Z2})R^{Z3}—, optionally substituted phenyl, or optionally substituted pyridyl;
each of R^{Z1} and R^{Z2} is, independently optionally substituted C1-C6 alkyl;
R^{Z3} is a covalent bond or an unsubstituted C1-C3 alkylene;
A is a covalent bond or an optionally substituted C1-C3 alkylene;
L is —CONR^{2A}(CH₂)ₒ or —R^{2A}NCO(CH₂)ₒ, wherein R^{2A} is H or optionally substituted C1-C6 alkyl, and o is 0, 1, or 2; and
R³ is selected from optionally substituted C1-C6 alkyl, optionally substituted alkaryl, optionally substituted alkheteroaryl, optionally substituted aryl, optionally substituted heteroaryl, optionally substituted C3-C9 cycloalkyl, and optionally substituted heterocyclyl.

2-3. (canceled)

4. The compound of claim 1, or a pharmaceutically acceptable salt, solvate, or prodrug thereof, or a stereoisomer thereof, wherein said compound has a structure according to the following formula, 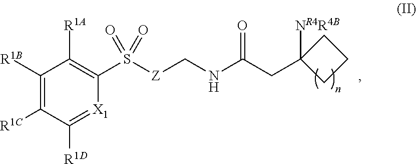
, wherein R^{4A} and R^{4B} are each, independently, H or optionally substituted C1-C6 alkyl, and n is an integer between 0-4.

5-9. (canceled)

10. The compound of claim 1, having a structure according to the following formula, 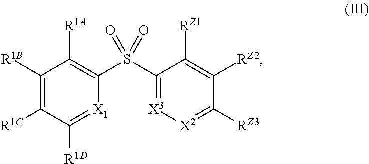
or a pharmaceutically acceptable salt, solvate, or prodrug thereof, or a stereoisomer thereof, wherein
X¹ is N or CR^{1E};
X² is N or CR^{Z4};
X³ is N or CR^{Z5};
each of R^{1A}, R^{1B}, R^{1C}, R^{1D}, R^{1E}, R^{Z4}, and R^{Z5} is selected, independently, from H, OH, optionally substituted C1-C6 alkyl, optionally substituted C2-C6 alkenyl, optionally substituted C2-C6 alkynyl, and optionally substituted C1-C6 alkoxy;
each of R^{Z1}, R^{Z2}, and R^{Z3} is selected, independently, from H, OH, optionally substituted C1-C6 alkyl, optionally substituted C2-C6 alkenyl, optionally substituted C2-C6 alkynyl, optionally substituted C1-C6 alkoxy, or the substructure ALR³, and wherein one and only one of R^{Z1}, R^{Z2}, and R^{Z3} is the substructure ALR³;
and
wherein no more than one of X² and X³ is N.

11-15. (canceled)

16. The compound of claim 10, or a pharmaceutically acceptable salt, solvate, or prodrug thereof, or a stereoisomer thereof, having a structure according to one of the following formulas, 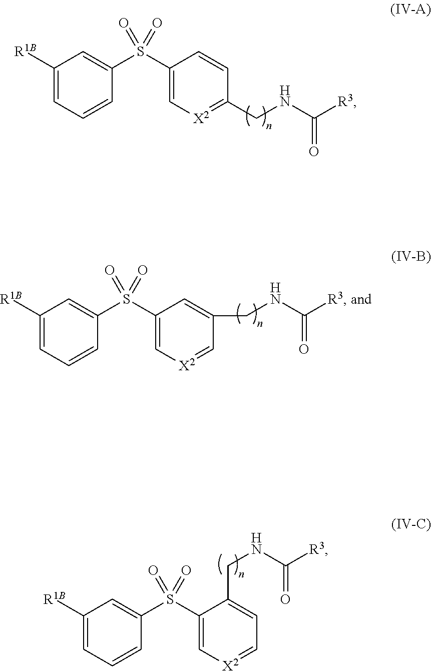
wherein
X² is N or CH;
R^{1B} is C1-C3 haloalkyl or C1-C3 haloalkoxy;
n is 1, 2, or 3; and
R³ is C1-C3 haloalkyl, optionally substituted heterocyclyl, optionally substituted aryl, optionally substituted heteroaryl, optionally substituted aryl, optionally substituted benzyl, or optionally substituted C3-C9 cycloalkyl.

17-23. (canceled)

24. The compound of claim 16, or a pharmaceutically acceptable salt, solvate, or prodrug thereof, or a stereoisomer thereof, having a structure according to the following formula, 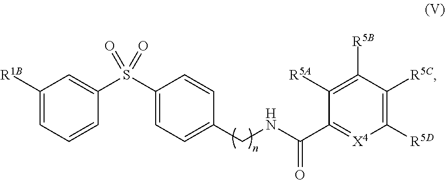
wherein
n is 1 or 2;
X⁴ is N or CH; and
each of R^{5A}, R^{5B}, R^{5C}, and R^{5D} is selected, independently, from H, F, Cl, C1-C3 haloalkyl, C1-C3 haloalkoxy, and SO₂(C1-C4 alkyl).

25-28. (canceled)

29. A compound having a structure according to the following formula, 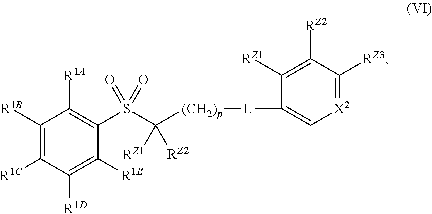
or a pharmaceutically acceptable salt, solvate, or prodrug thereof, or a stereoisomer thereof, wherein
p is 0, 1, 2, or 3;
L is —C(O)NR^{2A}- or —NR^{2A}C(O)—;
each of R^{Z1} and R^{Z2} is, independently, optionally substituted C1-C6 alkyl;
R^{2A} is H or optionally substituted C1-C6 alkyl;
each of R^{1A}, R^{1D}, and R^{1E} is selected, independently, from H, halogen, optionally substituted C1-C6 alkyl, and optionally substituted C1-C6 alkoxy;
R^{1B} is selected from optionally substituted C1-C6 alkyl or optionally substituted C1-C6 alkoxy;
R^{1C} is selected from H or halogen;
X² is N or CR^{Z4};
R^{Z4} is selected, independently, from H, halogen, optionally substituted C1-C6 alkyl, and optionally substituted C1-C6 alkoxy;
each of R^{Z1}, R^{Z2}, and R^{Z3} is selected, independently, from H or Ar¹, wherein one and only one of R^{Z1}, R^{Z2}, and R^{Z3} is Ar¹;
Ar¹ is 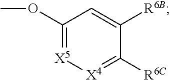
X⁴ is N or CR^{6D};
X⁵ is N or CR^{6E};
R^{6B}, R^{6D}, and R^{6E} are selected, independently, from H, halogen, optionally substituted C1-C6 alkyl, and optionally substituted C1-C6 alkoxy;
R^{6C} is selected from H or halogen; and
wherein no more than one of X² and X³ is N; and
wherein when o is 0, R^{Z1} and R^{Z2} are both CH₃, L is —CONH—, R^{1A}, R^{1D}, and R^{1E} are all H, R^{1B} is CF₃, R^{1B} is H, X¹ is N, and R^{Z1} and R^{Z2} are both H, Ar¹ is not O-(3-CF₃-4-FC₆H₃), O-(3-Cl-4-FC₆H₃), O-(6-CF₃-pyrid-3-yl), or O-(p-FC₆H₄); and
wherein when o is 0, 1, or 2, R^{Z1} and R^{Z2} are both CH₃, L is —CONH—, R^{1A} and R^{1E} are both H, R^{1B} is CF₃, R^{1C} is H, R^{1D} is H or F, X¹ is CH, and R^{Z1} and R^{Z2} are both H, Ar¹ is not O-(p-ClC₆H₄), OC₆H₅, or O-(p-FC₆H₄).

30. (canceled)

31. The compound of claim 29, wherein said compound has a structure according to the following formula, 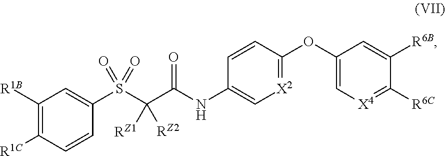
or a pharmaceutically acceptable salt, solvate, or prodrug thereof, or a stereoisomer thereof, wherein
R^{Z1} and R^{Z2} are each, independently, unsubstituted C1-C3 alkyl;
X² is CH or N;
X⁴ is CH or N;
R^{1B} is C1 haloalkyl or C1 haloalkoxy;
R^{1C} is H, Cl, or F; and
each of R^{6B} and R^{6C} is, independently, H, substituted C1 alkyl, or halogen.

32-36. (canceled)

37. A compound having a structure according to the following formula, 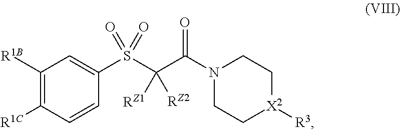
or a pharmaceutically acceptable salt, solvate, or prodrug thereof, or a stereoisomer thereof, wherein
each of R^{Z1} and R^{Z2} is selected, independently, from optionally substituted C1-C6 alkyl;
X² is CH or N;
R³ is optionally substituted aryl or optionally substituted heteroaryl; and
each of R^{1B} and R^{1C} is, independently, H, halogen, optionally substituted C1-C6 alkyl, or optionally substituted C1-C6 alkoxy.

38. (canceled)

39. A compound having a structure according to the following formula, 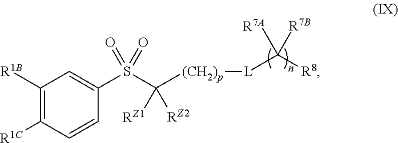
or a pharmaceutically acceptable salt, solvate, or prodrug thereof, or a stereoisomer thereof, wherein
n is an integer between 0-6, wherein n is not 0 when R⁸ is H or CF₃;
p is 0, 1, or 2;
L is —C(O)NR^{2A}- or —NR^{2A}C(O)—;
each of R^{Z1} and R^{Z2} is selected, independently, from optionally substituted C1-C6 alkyl;
R^{2A} is H or optionally substituted C1-C6 alkyl, or R^{2A} combines with R⁸ to form a heterocyclyl;
each of R^{1B} and R^{1C} is, independently, H, halogen, optionally substituted C1-C6 alkyl, or optionally substituted C1-C6 alkoxy;
each of R^{7A} and R^{7B} is, independently, H, OH, or optionally substituted C1-C6 alkyl;
R⁸ is H, CF₃, optionally substituted aryl, optionally substituted heteroaryl, optionally substituted arylsulfonyl, optionally substituted cycloalkyl, or optionally substituted heterocyclyl; wherein said optionally substituted groups are substituted with 1, 2, 3, 4, or 5 groups selected from halogen, OH, optionally substituted amino, optionally substituted C1-C6 alkyl, optionally substituted C1-C6 alkoxy, optionally substituted cycloalkyl, optionally substituted heterocyclyl, and —SO₂R⁹;
R⁹ is optionally substituted C1-C6 alkyl, optionally substituted aryl, or optionally substituted heterocyclyl; and wherein
when p is 0, n is 0, 1, or 2, R^{Z1} and R^{Z2} are both CH₃, L is —CONH— or —CONMe-, R^{1B} is CF₃, R^{1C} is H, and R^{7A} and R^{7B} are both H, R⁹ is not any of the following groups:
(a) a phenyl group that is substituted with 1 or 2 substituents selected from F, Cl, CF₃, or O^{t}Bu,
(b) a benzothiazole group substituted with one chloro group; or
(c) a benzimidazole group substituted with one CF₃ group;
when p is 1, n is 0, R^{Z1} and R^{Z2} are both CH₃, L is —NHCO—, R^{1B} is CF₃, R^{1C} is H, and R^{7A} and R^{7B} are both H, R⁹ is not any of the following groups:
(d) a phenyl group that substituted with 1 or 2 substituents selected from F, Cl, CF₃, SO₂Me, SO₂ⁱPr, or unsubstituted oxopyrrolidinyl, or a phenyl group that is substituted with two methyl groups and one methoxy group;
(e) a benzimidazole group substituted with one CF₃ or F group;
(f) an imidazol[1,2-a]pyridine group substituted with one CF₃ group;
(g) a pyridyl group substituted with one group selected from CF₃, CH₃, NHCO^{t}Bu, tert-butyl, and OCH₂CF₃, or a pyridyl group substituted with both a CF₃ group and a SO₂CH₃ group; and
when p is 2, n is 0, R^{Z1} and R^{Z2} are both CH₃, L is —CONH—, —NHCO—, or —NMeCO—, R^{1B} is CF₃, R^{1C} is H, and R^{7A} and R^{7B} are both H, R⁹ is not any of the following groups:
(h) a phenyl group that substituted with 1 or 2 substituents selected from F, Cl, CH₃, CF₃, OMe, SO₂Me, or SO₂ⁱPr;
(i) a pyrimidine group substituted with one CF₃ group, or substituted by both a methyl group and OⁱPr group;
(j) an imidazol[1,2-a]pyridine group substituted with one CF₃ group;
(k) a pyridyl group substituted with one CF₃, CH₃, tert-butyl, OCH₂CF₃, or pivalamido group, or a pyridyl group substituted with both a CF₃ group and a SO₂CH₃ or SO₂ⁱPr group, or both a Cl and OMe group; or
(l) a pyrazole group substituted by one CF₃ group, or by both one CF₃ and one CH₃ group.

40-43. (canceled)

44. A compound that is any of Compounds (1)-(227) of Tables 4 and 5, or a pharmaceutically acceptable salt, solvate, or prodrug thereof, or a stereoisomer thereof.

45-50. (canceled)

51. A method to treat a condition modulated by calcium channel activity, said method comprising administering to a subject in need of such treatment an effective amount of the compound of claim 44, or a pharmaceutically acceptable salt, solvate, or prodrug thereof, or a stereoisomer thereof, or a conjugate thereof.

52-55. (canceled)

56. The method of claim 51, wherein said condition is pain, epilepsy, Parkinson's disease, depression, psychosis, or tinnitus.

57-67. (canceled)
